(19) United States
(12) Patent Application Publication
(10) Pub. No.: US 2025/0111939 A1
(43) Pub. Date: Apr. 3, 2025

(54) BADGE AND PATIENT SUPPORT APPARATUS COMMUNICATION SYSTEM

(71) Applicant: Stryker Corporation, Kalamazoo, MI (US)

(72) Inventors: Madhu Sandeep Thota, Portage, MI (US); Krishna Sandeep Bhimavarapu, Kalamazoo, MI (US); Celso Henrique Farnese Pires Pereira, Portage, MI (US); Kirby M. Neihouser, Portage, MI (US); Jerald A. Trepanier, Augusta, MI (US); Madhu Thomas, London, Ontario (CA); Christopher P. Alvarez, Kalamazoo, MI (US); Anish Paul, Portage, MI (US); Michael W. Graves, Paw Paw, MI (US); Brianna R. Graves, Paw Paw, MI (US)

(21) Appl. No.: 18/725,394

(22) PCT Filed: Jun. 28, 2023
(86) PCT No.: PCT/US2023/026418

§ 371 Date: Jun. 28, 2024

Related U.S. Application Data
(60) Provisional application No. 63/356,061, filed on Jun. 28, 2022.
(60) Provisional application No. 63/356,065, filed on Jun. 28, 2022.
(60) Provisional application No. 63/356,238, filed on Jun. 28, 2022.
(60) Provisional application No. 63/357,363, filed on Jun. 30, 2022.

(51) Int. Cl. G16H 40/40; G16H 40/20; H04W 4/38

(52) U.S. Cl. CPC G16H 40/40; G16H 40/20; H04W 4/38

(57) ABSTRACT

A system includes a server and a patient support apparatus having a support surface, a plurality of ultra-wideband transceivers, and a controller. The controller is adapted to use the ultra-wideband transceivers to automatically determine a position of a badge and/or tag relative to the patient support apparatus and to use the position information of the badge and/or tag to automatically perform one or more of the following: display a cleaning screen used by cleaning workers; determine if the patient support apparatus is ready to be, or already has been, cleaned; identify and record an identity of the worker who cleaned the patient support apparatus; determine a closest transport worker; and/or update an equipment weight log maintained by the patient support apparatus. The badge(s) may be worn by a healthcare worker and the tag(s) may be coupled to an object or worn by the patient.

### CROSS-REFERENCE TO RELATED APPLICATIONS

This application claims priority to the following U.S. provisional patent applications: Ser. No. 63/356,065 filed Jun. 28, 2022, by inventors Jerald Trepanier et al. and entitled BADGE AND PATIENT SUPPORT APPARATUS COMMUNICATION SYSTEM; Ser. No. 63/356,061 filed Jun. 28, 2022, by inventors Krishna Sandeep Bhimavarapu et al. and entitled BADGE AND PATIENT SUPPORT APPARATUS COMMUNICATION SYSTEM; Ser. No. 63/356,238 filed Jun. 28, 2022, by inventors Madhu Sandeep Thota et al. and entitled BADGE AND PATIENT SUPPORT APPARATUS COMMUNICATION SYSTEM; and Ser. No. 63/357,363 filed Jun. 30, 2022, by inventors Madhu Sandeep Thota et al. and entitled BADGE AND PATIENT SUPPORT APPARATUS COMMUNICATION SYSTEM, the complete disclosures of all of which are incorporated herein by reference.

### BACKGROUND

The present disclosure relates to patient support apparatuses, such as beds, cots, stretchers, recliners, or the like. More specifically, the present disclosure relates to patient support apparatuses that communicate with one or more badges and/or other devices that are separate from the patient support apparatus itself.

### SUMMARY

According to the various aspects described herein, the present disclosure is directed to a patient support apparatus system that monitors the positions of badges worn by one or more different types of workers in a healthcare facility, and/or that monitors the positions of tags that may be worn by patients and/or coupled to devices. The system may include one or more servers and one more patient support apparatuses adapted to communicate with the server(s) and the badges.

The system helps to automate and/or reduce the labor involved in a number of tasks, such as, but not limited to, the following: cleaning the patient support apparatus; notifying the appropriate personnel when the patient support apparatus needs to be cleaned, and/or after it has been cleaned; verifying the cleaning of the patient support apparatus with the automatic identification of the person(s) who undertook the cleaning; notifying transport workers when a patient needs to be transported; determining a closest transport worker to a patient who needs transportation within the healthcare facility; updating an equipment weight log with an identification of equipment location, a time stamp of equipment changes, and an ID of the caregiver responsible for the equipment changes. These and other aspects of the present disclosure will be apparent to a person of ordinary skill light of the following written description and accompanying drawings.

According to a first aspect of the present disclosure, a patient support apparatus system is provided that includes a patient support apparatus and a server. The patient support apparatus includes a support surface adapted to support a patient; a plurality of ultra-wideband transceivers; a display; a network transceiver adapted to communicate with the server; and a controller. The controller is adapted to use radio frequency (RF) communication between the plurality of ultra-wideband transceivers and a badge worn by a worker to determine a position of the badge relative to the patient support apparatus. The controller is further adapted to receive a badge identifier from the badge; to determine if the badge is positioned inside or outside of a volume of space; and, if the badge is positioned inside the volume of space, to transmit the badge identifier to the server. The server is adapted to perform the following: determine if the badge is associated with a cleaning worker, and if the badge is associated with a cleaning worker, to instruct the controller to automatically display a cleaning screen on the display.

According to another aspect of the present disclosure, the cleaning screen includes instructions for cleaning the patient support apparatus.

In some aspects, the controller and/or the server is adapted to perform the following: (i) measure a length of time the badge remains inside the volume of space; (ii) compare the measured length of time to a threshold amount of time; and (iii) if the measured length of time exceeds the threshold amount of time, determine that the patient support apparatus has been cleaned.

In some aspects, after the patient support apparatus has been determined to have been cleaned, the controller is adapted to determine if a scale system onboard the patient support apparatus has been zeroed or not.

The controller, in some aspects, the controller is further adapted to automatically display a bed zeroing reminder screen on the display after the badge has been determined to be associated with a cleaning worker.

In some aspects, the controller is adapted to automatically display the bed zeroing reminder screen on the display a predetermined amount of time after the badge has been determined to be associated with a cleaning worker.

In some aspects, the controller is adapted to display the bed zeroing reminder screen on the display a predetermined amount of time after the patient support apparatus has been determined to have been cleaned.

The controller and/or the server, in some aspects, is adapted to automatically send a reminder message to the badge if the scale system onboard the patient support apparatus has not been zeroed.

The controller and/or the server, in some aspects, is further adapted to send the reminder message a predetermined amount of time after the patient support apparatus has been determined to have been cleaned.

The controller and/or the server, in some aspects, is further adapted to send the reminder message a predetermined amount of time after the badge has been determined to be associated with a cleaning worker.

The controller, in some aspects, is further adapted to record an identity of the cleaning worker.

In some aspects, the controller is further adapted to display on the display the identity of the cleaning worker.

The controller, in some aspects, is further adapted to cause cleaning instructions to be aurally emitted from a speaker onboard the patient support apparatus.

After the patient support apparatus has been determined to have been cleaned, the server is further adapted, in some aspects, to add an identifier of the patient support apparatus to a list of cleaned patient support apparatuses.

The controller and/or the server, in some aspects, is adapted to determine that the patient support apparatus has been cleaned only if an additional condition has been met in addition to the measured length of time exceeding the threshold amount of time.

The additional condition, in some aspects, is one or more of the following: (a) a lack of patient weight detected by a scale system integrated into the patient support apparatus; (b) a lack of an ultra-wideband tag worn by the patient being detected inside the volume of space by the ultra-wideband transceivers; (c) the receipt of data from an Admission, Discharge and Transfer server indicating that the patient assigned to the patient support apparatus has been discharged from a healthcare facility in which the patient support apparatus is located; and/or (d) a detection of a second badge inside the volume of space within a defined amount of time prior to the badge being positioned inside the volume of space, wherein the second badge is associated with a transport worker responsible for transporting patients.

According to another aspect of the present disclosure, a patient support apparatus system is provided that includes a patient support apparatus and a server. The patient support apparatus includes a support surface adapted to support a patient; a plurality of ultra-wideband transceivers; a display; a network transceiver adapted to communicate with the server; and a controller. The controller is adapted to use radio frequency (RF) communication between the plurality of ultra-wideband transceivers and a badge worn by a worker to determine a position of the badge relative to the patient support apparatus. The controller is further adapted to receive a badge identifier from the badge; to determine if the badge is positioned inside or outside of a volume of space; and, if the badge is positioned inside the volume of space, to transmit the badge identifier to the server. The server is adapted to perform the following: (i) determine if the badge is associated with a cleaning worker; (ii) measure a length of time the badge remains inside the volume of space; (iii) compare the measured length of time to a threshold amount of time; and (iv) if the measured length of time exceeds the threshold amount of time, determine that the patient support apparatus has been cleaned.

In some aspects, if the badge is associated with a cleaning worker, the controller and/or the server is further adapted to automatically display a cleaning screen on the display.

The cleaning screen, in some aspects, includes instructions for cleaning the patient support apparatus.

In some aspects, after the patient support apparatus has been determined to have been cleaned, the controller is adapted to determine if a scale system onboard the patient support apparatus has been zeroed or not.

In some aspects, after the badge has been determined to be associated with a cleaning worker, the controller is further adapted to automatically display a bed zeroing reminder screen on the display.

The controller, in some aspects, is adapted to automatically display the bed zeroing reminder screen on the display a predetermined amount of time after the badge has been determined to be associated with a cleaning worker.

The controller, in some aspects, is adapted to display the bed zeroing reminder screen on the display a predetermined amount of time after the patient support apparatus has been determined to have been cleaned.

The controller and/or the server, in some aspects, is adapted to automatically send a reminder message to the badge if the scale system onboard the patient support apparatus has not been zeroed.

The controller and/or server, in some aspects, is further adapted to send the reminder message a predetermined amount of time after the patient support apparatus has been determined to have been cleaned.

In some aspects, the patient support apparatus further includes a speaker, and the controller is further adapted to cause cleaning instructions to be aurally emitted from the speaker.

The controller, in some aspects, is further adapted to record an identity of the cleaning worker.

The controller, in some aspects, is further adapted to display on the display the identity of the cleaning worker.

In some aspects, after the patient support apparatus has been determined to have been cleaned, the server is further adapted to add an identifier of the patient support apparatus to a list of cleaned patient support apparatuses.

The controller and/or the server, in some aspects, is adapted to determine that the patient support apparatus has been cleaned only if an additional condition has been met in addition to the measured length of time exceeding the threshold amount of time.

The additional condition may be one or more of the following: a lack of patient weight detected by a scale system integrated into the patient support apparatus; the receipt of data from an Admission, Discharge and Transfer server indicating that the patient assigned to the patient support apparatus has been discharged from a healthcare facility in which the patient support apparatus is located; a lack of an ultra-wideband tag worn by the patient being detected inside the volume of space by the ultra-wideband transceivers; and/or a detection of a second badge inside the volume of space within a defined amount of time prior to the badge being positioned inside the volume of space.

The second badge, in some aspects, is associated with a transport worker responsible for transporting patients.

According to another aspect of the present disclosure, a patient support apparatus system is provided that includes a server and a patient support apparatus. The patient support apparatus includes a support surface adapted to support a patient; a plurality of ultra-wideband transceivers; a network transceiver adapted to communicate with the server; and a controller. The controller is adapted to use radio frequency (RF) communication between the plurality of ultra-wideband transceivers and a badge worn by a worker to determine a position of the badge relative to the patient support apparatus. The controller is further adapted to receive a badge identifier from the badge; to determine if the badge is positioned inside or outside of a volume of space; and, if the badge is positioned inside the volume of space, to transmit the badge identifier to the server. The server and/or the controller is adapted to perform the following: (i) determine if the badge is associated with a transport worker; and (ii) if the badge is associated with a transport worker, determine that the patient support apparatus is ready to be cleaned.

According to other aspects of the present disclosure, the controller and/or the server may be adapted to determine that the patient support apparatus is ready to be cleaned only if an additional condition has been met.

In some aspects, the controller and/or the server is further adapted to measure a length of time the badge remains within the volume of space and to compare the measured length of time to a threshold amount of time. The additional condition may be the measured length of time exceeding the threshold amount of time.

In some aspects, the additional condition is any one or more of the following: a lack of patient weight detected by a scale system integrated into the patient support apparatus; a lack of an ultra-wideband tag worn by the patient being detected within the volume of space by the ultra-wideband transceivers; the receipt of data from an Admission, Discharge and Transfer server indicating that the patient assigned to the patient support apparatus has been discharged from a healthcare facility in which the patient support apparatus is located; and/or a lack of the badge in the volume of space.

In some aspects, the server is further configured to send a message to a cleaning server after the patient support apparatus has been determined to be ready to be cleaned, and/or to send a message to a second badge that is associated with a cleaning worker responsible for cleaning the patient support apparatus.

In some aspects, the server is further configured to automatically transmit a message to the badge after the badge has moved outside of the volume of space, wherein the message identifies a transportation assignment for the worker.

According to yet another aspect of the present disclosure, a patient support apparatus system is provided that includes a server and a patient support apparatus. The patient support apparatus includes a support surface adapted to support a patient; a plurality of ultra-wideband transceivers; a network transceiver adapted to communicate with the server; and a controller. The controller is adapted to use radio frequency (RF) communication between the plurality of ultra-wideband transceivers and a tag worn by the patient to determine a position of the tag relative to the patient support apparatus. The controller is further adapted to receive a tag identifier from the tag; to determine if the tag is positioned inside or outside of a volume of space; and, if the tag is positioned outside the volume of space, to transmit a message to the server. The controller and/or the server is adapted to determine that the patient support apparatus is ready to be cleaned.

In some aspects, the controller and/or the server is adapted to determine that the patient support apparatus is ready to be cleaned only if an additional condition has been met.

In some aspects, the server and/or controller is further adapted to measure a length of time the tag remains outside the volume of space and compare the measured length of time to a threshold amount of time, and the additional condition is the measured length of time exceeding the threshold amount of time.

The additional condition, in some aspects, is any one or more of the following: presence of a badge inside the volume of space, wherein the badge is associated with a transport worker; a lack of patient weight detected by a scale system integrated into the patient support apparatus; and/or the receipt of data from an Admission, Discharge and Transfer server indicating that the patient assigned to the patient support apparatus has been discharged from a healthcare facility in which the patient support apparatus is located.

In some aspects, the server is further configured to send a message to a cleaning server after the patient support apparatus has been determined to be ready to be cleaned.

The server, in some aspects, is further configured to send a message to a second badge, wherein the second badge is associated with a cleaning worker responsible for cleaning the patient support apparatus.

In some aspects, the server is further configured to automatically transmit a message to the badge after the badge has moved outside of the volume of space, wherein the message identifies a transportation assignment for the transport worker.

According to still another aspect of the present disclosure, a patient support apparatus system is provided that includes a patient support apparatus and a server. The patient support apparatus includes a support surface adapted to support a patient; a scale system adapted to detect a weight of the patient; a plurality of ultra-wideband transceivers; a network transceiver adapted to communicate with the server; and a controller. The controller is adapted to use radio frequency (RF) communication between the plurality of ultra-wideband transceivers and a badge worn by a worker to determine a position of the badge relative to the patient support apparatus. The controller is further adapted to receive a badge identifier from the badge; to determine if the badge is positioned inside or outside of a volume of space; and, if both the badge is positioned inside the volume of space and the patient weight is removed from the patient support apparatus, to transmit a message to the server. The server and/or the controller is adapted to determine that the patient support apparatus is ready to be cleaned.

In some aspects, the server is further configured to send a message to a cleaning server after the patient support apparatus has been determined to be ready to be cleaned.

The server, in some aspects, is further configured to send a message to a second badge, wherein the second badge is associated with a cleaning worker responsible for cleaning the patient support apparatus.

The controller and/or the server may, in some aspects, be adapted to determine that the patient support apparatus is ready to be cleaned only if an additional condition has been met, wherein the additional condition is one or more both of the following: the badge being associated with a transport worker; and/or the receipt of data from an Admission, Discharge and Transfer server indicating that the patient assigned to the patient support apparatus has been scheduled to be transported to a location within a healthcare facility in which the patient support apparatus is located.

In some aspects, the controller is further adapted to use radio frequency (RF) communication between the plurality of ultra-wideband transceivers and a second badge worn by a second worker to determine a position of the second badge relative to the patient support apparatus. The controller may be further adapted to receive a second badge identifier from the second badge; to determine if the second badge is positioned inside or outside of the volume of space; and, if the second badge is positioned inside the volume of space, to transmit the second badge identifier to the server. The controller and/or the server may further be adapted to perform the following: (i) determine if the second badge is associated with a cleaning worker; and (ii) if the second badge is associated with a cleaning worker, automatically take at least one additional action.

In some aspects, the additional action includes the following: (a) measuring a length of time the second badge remains inside the volume of space; (b) comparing the measured length of time to a threshold amount of time; and (c) if the measured length of time exceeds the threshold amount of time, determining that the patient support apparatus has been cleaned.

In some aspects, the additional action includes displaying a cleaning screen on the display.

According to still another aspect of the present disclosure, a patient support apparatus system is provided that includes a patient support apparatus and a server. The patient support apparatus includes a support surface adapted to support a patient; a plurality of ultra-wideband transceivers; a display; a network transceiver adapted to communicate with the server; and a controller. The controller is adapted to use radio frequency (RF) communication between the plurality of ultra-wideband transceivers and a badge worn by a worker to determine a position of the badge relative to the patient support apparatus. The controller is further adapted to receive a badge identifier from the badge; to determine if the badge is positioned inside or outside of a volume of space; and, if the badge is positioned inside the volume of space, to transmit the badge identifier to the server. The server and/or the controller is adapted to receive data indicating that the patient support apparatus has been cleaned; to determine if the badge is associated with a cleaning worker; and, if the badge is associated with a cleaning worker, to record an identity of the cleaning worker.

In some aspects, the controller is further adapted to display the identity of the cleaning worker on the display.

The controller and/or the server, in some aspects, is adapted to record a time at which the badge is positioned inside of the volume of the space.

The controller, in some aspects, is further adapted to display the time on the display.

In some aspects, after receiving the data indicating that the patient support apparatus has been cleaned, the controller is adapted to determine if a scale system onboard the patient support apparatus has been zeroed or not.

In some aspects, if the scale system has not been zeroed, the controller is further adapted to automatically display a bed zeroing reminder screen on the display.

The controller, in some aspects, is adapted to automatically display the bed zeroing reminder screen on the display a predetermined amount of time after the badge has been determined to be inside the volume of space.

The controller and/or the server, in some aspects, is adapted to automatically send a reminder message to the badge if the scale system onboard the patient support apparatus has not been zeroed.

The controller and/or the server, in some aspects, is further adapted to send the reminder message after the patient support apparatus has been determined to have been cleaned.

In some aspects, the controller and/or the server, is further adapted to send the reminder message after the badge has been determined to be associated with a cleaning worker.

According to still another aspects of the present disclosure, a patient support apparatus system is provided that includes a plurality of patient support apparatuses and a server. Each of the patient support apparatus includes a support surface adapted to support a patient; a plurality of ultra-wideband transceivers; a network transceiver adapted to communicate with a healthcare facility computer network; and a controller. Each controller is adapted to use radio frequency (RF) communication between the plurality of ultra-wideband transceivers and a badge to determine a position of the badge relative to the patient support apparatus. Each controller is further adapted to receive a badge identifier from the badge and to transmit the badge identifier to the server. The server is adapted to receive the badge identifiers from the plurality of patient support apparatuses; to determine which badge identifiers are associated with transport workers; to receive data indicating that a particular patient has been discharged from the healthcare facility; and to determine a closest transport worker badge that is associated with a transport worker and that is currently positioned closest to a room in which the particular patient is located.

The server, in some aspects, is further adapted to automatically send a message to the closest transport worker badge.

The server, in some aspects, is further adapted to determine if the transport worker associated with the closest transport worker badge is currently working on a transportation assignment or not.

In some aspects, the server is further adapted to determine a second closest transport worker badge that is associated with a transport worker and that is currently positioned second closest to the room in which the particular patient is located.

The server, in some aspects, is further configured to determine that a particular patient support apparatus assigned to the particular patient is ready to be cleaned.

The server, in some aspects, is configured to determine that the particular patient support apparatus is ready to be cleaned only after receiving a message from the particular patient support apparatus indicating that a weight of the particular patient has been removed from the particular patient support apparatus.

In some aspects, the server is further configured to send a message to a cleaning server after the particular patient support apparatus has been determined to be ready to be cleaned.

In some aspects, the server is further configured to send a message to a second badge, wherein the second badge is associated with a cleaning worker responsible for cleaning the particular patient support apparatus.

According to yet another aspect of the present disclosure, a patient support apparatus system is provided that includes a patient support apparatus and a server. The patient support apparatus includes a support surface adapted to support a patient; a plurality of ultra-wideband transceivers; a display; a scale system; a network transceiver adapted to communicate with the server; and a controller. The controller is adapted to use radio frequency (RF) communication between the plurality of ultra-wideband transceivers and a tag attached to an object to determine a position of the tag relative to the patient support apparatus. The controller further adapted to use the relative position of the tag and outputs from the scale system to determine if the object has been placed on the patient support apparatus.

The controller, in some aspects, is further adapted to use the position of the tag relative to the patient support apparatus to determine where the object has been placed on the patient support apparatus.

In some aspects, the controller is further adapted to use the outputs from the scale system to determine where the object has been placed on the patient support apparatus.

The controller, in some aspects, is further adapted to add the object to a weight log maintained in a memory of the patient support apparatus.

In some aspects, the controller is further adapted to display on the display a list of objects currently in the weight log.

The controller, in some aspects, is further adapted to display on the display a location of each of the objects in the weight log.

The controller, in some aspects, is further adapted to display on the display a time at which each object in the weight log was added to the weight log.

In some aspects, the controller is further adapted to perform the following: to use radio frequency (RF) communication between the plurality of ultra-wideband transceivers and a badge worn by a caregiver to determine a position of the badge relative to the patient support apparatus; to determine if the badge is positioned inside or outside of a volume of space when the object is placed on the patient support apparatus; and, if the badge is positioned inside the volume of space when the object is placed on the patient support apparatus, to record an identity of the caregiver associated with the badge.

The controller, in some aspects, is further adapted to add to the weight log the identity of the caregiver associated with the badge.

In some aspects, the controller is further adapted to perform the following: to use radio frequency (RF) communication between the plurality of ultra-wideband transceivers and a second badge worn by a second caregiver to determine a position of the second badge relative to the patient support apparatus; to determine if the second badge is positioned inside or outside of a volume of space; and to automatically display the weight log on the display if the second caregiver associated with the second badge has not previously viewed the weight log within a defined time period.

The defined time period, in some aspects, is equal to a time between nursing shift changes for the healthcare facility in which the patient support apparatus is located.

According to yet another aspect of the present disclosure, a patient support apparatus is provided that includes a support surface adapted to support a patient, a plurality of ultra-wideband transceivers, a scale system adapted to measure an amount of weight supported by the support surface; and a controller. The controller is adapted to use radio frequency (RF) communication between the plurality of ultra-wideband transceivers and a tag worn by an individual to determine a position of the tag relative to the patient support apparatus. The controller is further adapted to receive a tag identifier from the tag, to determine if the tag is positioned inside or outside of a volume of space, and, if the tag is positioned outside of the volume of space, to automatically zero the scale system.

According to yet another aspect of the present disclosure, a patient support apparatus is provided that includes a support surface adapted to support a patient thereon, a plurality of ultra-wideband transceivers, a scale system adapted to measure an amount of weight supported by the support surface, and a controller. The scale system includes a manual zeroing control adapted, when activated, to zero the scale system. The controller is adapted to use radio frequency (RF) communication between the plurality of ultra-wideband transceivers and a tag worn by an individual to determine a position of the tag relative to the patient support apparatus. The controller is further adapted to receive a tag identifier from the tag, to determine if the tag identifier corresponds to an authorized individual or an unauthorized individual, to determine if the tag is positioned inside or outside of a volume of space, and if the tag corresponds to an unauthorized individual and is positioned inside the volume of space, to disable the manual zeroing control.

According to other aspects of the present disclosure, the controller is further adapted to use the tag identifier to determine a classification of the individual.

In some aspects, the classification includes at least one of the following: a patient, a caregiver, a cleaner, a transport worker, or a service technician.

In some aspects, the controller is further adapted to use the tag identifier to determine if the individual wearing the tag is a patient or a caregiver.

The controller, in some aspects, is further adapted to prevent any user from manually zeroing the scale system if the tag is positioned inside of the volume of space and the individual wearing the tag is the patient or the caregiver.

In some aspects, the patient support apparatus further includes a wheel and a brake adapted to selectively lock and unlock the wheel, and the controller is further adapted to automatically zero the scale system if at least the following two conditions are both true: (1) the brake has been activated, and (2) the tag is positioned outside of the volume of space.

The controller, in some aspects, is adapted to automatically zero the scale system if at least the following two conditions are both true: (1) the scale system detects no weight changes above a first threshold for a time period greater than a second threshold, and (2) the tag is positioned outside of the volume of space.

The controller, in some aspects, is adapted to automatically zero the scale system if at least the following two conditions are both true: (1) a cable of the patient support apparatus is plugged into an outlet, and (2) the tag is positioned outside of the volume of space.

In some aspects, the cable is a power cable adapted to plug into a power outlet.

In some aspects, the controller is adapted to automatically zero the scale system if at least the following two conditions are both true: (1) a height of the support surface has been raised past a threshold, and (2) the tag is positioned outside of the volume of space.

The patient support apparatus, in some aspects, further includes an obstruction detection sensor adapted to detect a collision with an obstruction when a component of the patient support apparatus moves, and the controller is further adapted to automatically zero the scale system if at least the following two conditions are both true: (1) the obstruction detection sensor is not detecting a collision with an obstruction, and (2) the tag is positioned outside of the volume of space.

In some aspects, the controller is adapted to automatically zero the scale system if at least the following two conditions are both true: (1) the tag is positioned outside of the volume of space, and (2) the scale system detects a weight of more than a threshold amount.

The threshold amount, in some aspects, is at least approximately 100 milligrams.

The controller, in some aspects, is adapted to automatically zero the scale system if at least the following two conditions are both true: (1) the tag is positioned outside of the volume of space, and (2) the scale system detects a weight of more than a first threshold amount but less than a second threshold amount.

In some aspects, the controller is adapted to automatically zero the scale system if at least the following two conditions are both true: (1) the tag is positioned outside of the volume of space, and (2) the scale system detects a weight of less than a threshold amount.

The threshold amount, in some aspects, is more than approximately fifteen kilograms.

In some aspects, the controller is further adapted to use radio frequency (RF) communication between the plurality of ultra-wideband transceivers and a second tag worn by a second individual to determine a position of the second tag relative to the patient support apparatus. The controller is further adapted to receive a second tag identifier from the second tag, to determine if the second tag is positioned inside or outside of the volume of space, and, if both the tag is positioned outside the volume of space and the second tag is positioned inside the volume of space, to allow the second individual to manually zero the scale system.

In some aspects, the controller is further adapted to use the second tag identifier to determine if the second individual wearing the second tag is authorized to manually zero the scale system.

The controller, in some aspects of the present disclosure, is further adapted to enable the manual zeroing control if the tag corresponds to an authorized individual and is positioned inside the volume of space.

In some aspects, the controller is further adapted to disable the manual zeroing control if the tag corresponds to an authorized individual and is positioned outside the volume of space.

The controller, in some aspects, is further adapted to disable the manual zeroing control if the tag corresponds to an unauthorized individual and is positioned outside the volume of space.

In some aspects, the controller is further adapted to automatically zero the scale system if the tag is positioned outside of the volume of space.

Before the various aspects of the disclosure are explained in detail, it is to be understood that the claims are not to be limited to the details of operation or to the details of construction and the arrangement of the components set forth in the following description or illustrated in the drawings. The aspects described herein are capable of being practiced or being carried out in alternative ways not expressly disclosed herein. Also, it is to be understood that the phraseology and terminology used herein are for the purpose of description and should not be regarded as limiting. The use of “including” and “comprising” and variations thereof is meant to encompass the items listed thereafter and equivalents thereof as well as additional items and equivalents thereof. Further, enumeration may be used in the description of various embodiments. Unless otherwise expressly stated, the use of enumeration should not be construed as limiting the claims to any specific order or number of components. Nor should the use of enumeration be construed as excluding from the scope of the claims any additional steps or components that might be combined with or into the enumerated steps or components.

### BRIEF DESCRIPTION OF THE DRAWINGS

FIG. 1 is a perspective view of a patient support apparatus according to a first aspect of the present disclosure;

FIG. 2 is a plan view of an illustrative caregiver control panel of the patient support apparatus of FIG. 1;

FIG. 3 is a plan view of an illustrative patient control panel of the patient support apparatus of FIG. 1;

FIG. 4 is a perspective view of a patient support apparatus system of the present disclosure showing the patient support apparatus, a device, a display device, a caregiver with a badge, and a linked locator unit that is used for automatically detecting the location of a patient support apparatus;

FIG. 5 is a block diagram of the patient support apparatus system of FIG. 4;

FIG. 6 is a plan view of an illustrative healthcare facility area, a patient support apparatus, a plurality of caregiver badges, a device, a plurality of space volumes, and a plurality of wall units;

FIG. 7 is a block diagram of an unlinked locator unit, the healthcare facility network, and a caregiver badge;

FIG. 8 is a diagram of the system of patient support apparatus system of FIG. 4 showing more details of some of the data contained in a plurality of servers that may be resident in the healthcare facility;

FIG. 9 is an example of a dashboard screen that may be displayed on a remote electronic device that shows the status of multiple patient support apparatuses, medical devices, and activities performed by caregivers, and other information;

FIG. 10 is example of a bed cleaned message that may be displayed on a display of the present disclosure;

FIG. 11 is an example of a bed zeroing reminder message that may be displayed on a display of the present disclosure;

FIG. 12 is an example of a cleaning instructions screen that may be displayed on a display of the present disclosure;

FIG. 13 is an example of an equipment weight log screen that may be displayed on a display of the present disclosure; and

FIG. 14 is an example of a scale screen that may be displayed on the patient support apparatus.

### DETAILED DESCRIPTION OF THE EMBODIMENTS

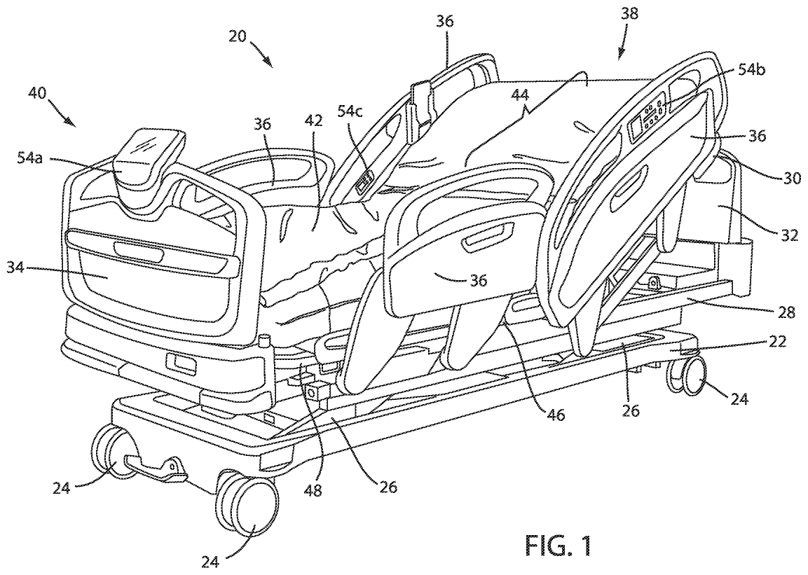

An illustrative patient support apparatus 20 according to an embodiment of the present disclosure is shown in FIG. 1. Although the particular form of patient support apparatus 20 illustrated in FIG. 1 is a bed adapted for use in a hospital or other medical setting, it will be understood that patient support apparatus 20 could, in different embodiments, be a cot, a stretcher, a recliner, or any other structure capable of supporting a patient in a healthcare environment.

In general, patient support apparatus 20 includes a base 22 having a plurality of wheels 24, a pair of lifts 26 supported on the base 22, a litter frame 28 supported on the lifts 26, and a support deck 30 supported on the litter frame 28. Patient support apparatus 20 further includes a headboard 32, a footboard 34 and a plurality of siderails 36. Siderails 36 are all shown in a raised position in FIG. 1 but are each individually movable to a lower position in which ingress into, and egress out of, patient support apparatus 20 is not obstructed by the lowered siderails 36.

Lifts 26 are adapted to raise and lower litter frame 28 with respect to base 22. Lifts 26 may be hydraulic actuators, electric actuators, or any other suitable device for raising and lowering litter frame 28 with respect to base 22. In the illustrated embodiment, lifts 26 are operable independently so that the tilting of litter frame 28 with respect to base 22 can also be adjusted, to place the litter frame 28 in a flat or horizontal orientation, a Trendelenburg orientation, or a reverse Trendelenburg orientation. That is, litter frame 28 includes a head end 38 and a foot end 40, each of whose height can be independently adjusted by the nearest lift 26. Patient support apparatus 20 is designed so that when an occupant lies thereon, his or her head will be positioned adjacent head end 38 and his or her feet will be positioned adjacent foot end 40.

Litter frame 28 provides a structure for supporting support deck 30, the headboard 32, footboard 34, and siderails 36. Support deck 30 provides a support surface for a mattress 42, or other soft cushion, so that a person may lie and/or sit thereon. In some embodiments, the mattress 42 includes one or more inflatable bladders that are controllable via a blower, or other source of pressurized air. In at least one embodiment, the inflation of the bladders of the mattress 42 is controllable via electronics built into patient support apparatus 20. In one such embodiments, mattress 42 may take on any of the functions and/or structures of any of the mattresses disclosed in commonly assigned U.S. Pat. No. 9,468,307 issued Oct. 18, 2016, to inventors Patrick Lafleche et al., the complete disclosure of which is incorporated herein by reference. Still other types of mattresses may be used.

Support deck 30 is made of a plurality of sections, some of which are pivotable about generally horizontal pivot axes. In the embodiment shown in FIG. 1, support deck 30 includes at least a head section 44, a thigh section 46, and a foot section 48, all of which are positioned underneath mattress 42 and which generally form flat surfaces for supporting mattress 42. Head section 44, which is also sometimes referred to as a Fowler section, is pivotable about a generally horizontal pivot axis between a generally horizontal orientation (not shown in FIG. 1) and a plurality of raised positions (one of which is shown in FIG. 1). Thigh section 46 and foot section 48 may also be pivotable about generally horizontal pivot axes.

In some embodiments, patient support apparatus 20 may be modified from what is shown to include one or more components adapted to allow the user to extend the width and/or length of patient support deck 30, thereby allowing patient support apparatus 20 to accommodate patients of varying sizes. When so modified, the width of deck 30 may be adjusted sideways and/or lengthwise in increments or otherwise.

As used herein, the term “longitudinal” refers to a direction parallel to an axis between the head end 38 and the foot end 40. The terms “transverse” or “lateral” refer to a direction perpendicular to the longitudinal direction and parallel to a surface on which the patient support apparatus 20 rests.

It will be understood by those skilled in the art that patient support apparatus 20 can be designed with other types of mechanical constructions that are different from what is shown in the attached drawings, such as, but not limited to, the construction described in commonly assigned, U.S. Pat. No. 10,130,536 to Roussy et al., entitled PATIENT SUPPORT USABLE WITH BARIATRIC PATIENTS, the complete disclosure of which is incorporated herein by reference. In another embodiment, the mechanical construction of patient support apparatus 20 may include the same, or nearly the same, structures as the Model 3002 S3 bed manufactured and sold by Stryker Corporation of Kalamazoo, Michigan. This construction is described in greater detail in the Stryker Maintenance Manual for the MedSurg Bed, Model 3002 S3, published in 2010 by Stryker Corporation of Kalamazoo, Michigan, the complete disclosure of which is incorporated herein by reference. In still another embodiment, the mechanical construction of patient support apparatus 20 may include the same, or nearly the same, structure as the Model 3009 Procuity MedSurg bed manufactured and sold by Stryker Corporation of Kalamazoo, Michigan. This construction is described in greater detail in the Stryker Maintenance Manual for the 3009 Procuity MedSurg bed (publication 3009-009-002, Rev. A.0), published in 2020 by Stryker Corporation of Kalamazoo, Michigan.

It will be understood by those skilled in the art that patient support apparatus 20 can be designed with still other types of mechanical constructions, such as, but not limited to, those described in commonly assigned, U.S. Pat. No. 7,690,59 issued Apr. 6, 2010, to Lemire et al., and entitled HOSPITAL BED; and/or commonly assigned U.S. Pat. publication No. 2007/0163045 filed by Becker et al. and entitled PATIENT HANDLING DEVICE INCLUDING LOCAL STATUS INDICATION, ONE-TOUCH FOWLER ANGLE ADJUSTMENT, AND POWER-ON ALARM CONFIGURATION, the complete disclosures of both of which are also hereby incorporated herein by reference. The overall mechanical construction of patient support apparatus 20 may also take on still other forms different from what is disclosed in the aforementioned references provided the patient support apparatus includes one or more of the functions, features, and/or structures discussed in greater detail below.

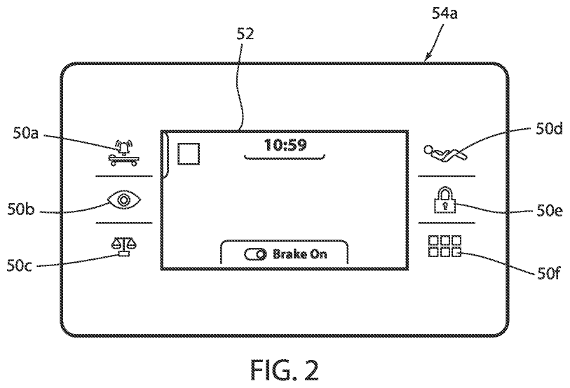

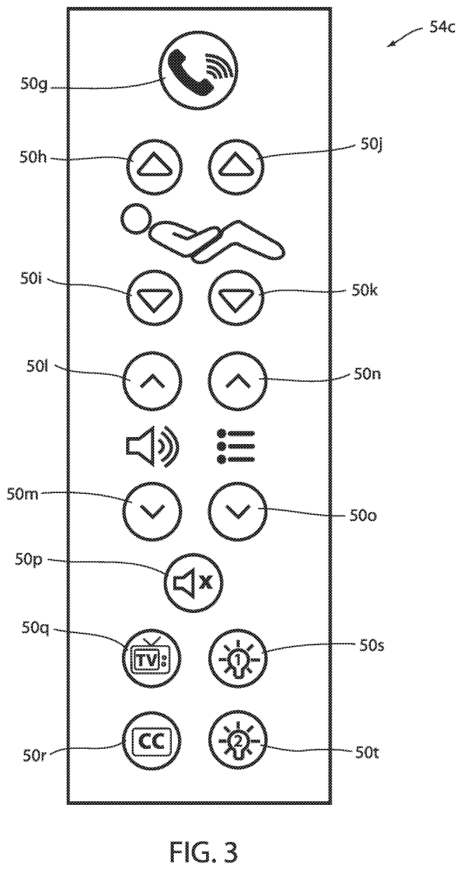

Patient support apparatus 20 further includes a plurality of control panels 54 that enable a user of patient support apparatus 20, such as a patient and/or an associated caregiver, to control one or more aspects of patient support apparatus 20. In the embodiment shown in FIG. 1, patient support apparatus 20 includes a footboard control panel 54a, a pair of outer siderail control panels 54b (only one of which is visible), and a pair of inner siderail control panels 54c (only one of which is visible). Footboard control panel 54a and outer siderail control panels 54b are intended to be used by caregivers, or other authorized personnel, while inner siderail control panels 54c are intended to be used by the patient associated with patient support apparatus 20. Each of the control panels 54 includes a plurality of controls 50 (see, e.g. FIGS. 2-3), although each control panel 54 does not necessarily include the same controls and/or functionality.

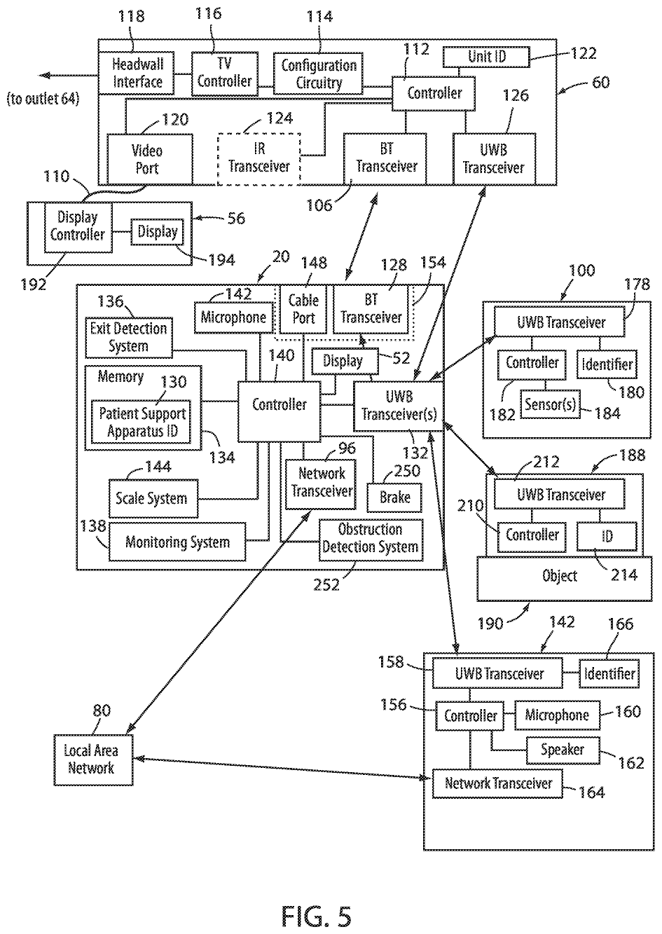

Among other functions, controls 50 of control panel 54a allow a user to control one or more of the following: change a height of support deck 30; raise or lower head section 44; activate and deactivate a brake 250 for wheels 24; arm and disarm an exit detection system 136 and/or an onboard monitoring system 138 (FIG. 5); change various settings on patient support apparatus 20; view the current location of the patient support apparatus 20 as determined by the location detection system discussed herein; view what devices, such as, but not limited to, medical devices, exercise devices, patient tags, nurse call devices, healthcare worker badges, etc. that the patient support apparatus 20 has associated itself with; and perform other actions. One or both of the inner siderail control panels 54c also include at least one control that enables a patient to call a remotely located nurse (or other caregiver). In addition to the nurse call control, one or both of the inner siderail control panels 54c also include one or more controls for controlling one or more features of one or more room devices positioned within the same room as the patient support apparatus 20. As will be described in more detail below, such room devices include, but are not necessarily limited to, a television, a reading light, and a room light. With respect to the television, the features that may be controllable by one or more controls 50 on control panel 54c include, but are not limited to, the volume, the channel, the closed-captioning, and/or the power state of the television. With respect to the room and/or night lights, the features that may be controlled by one or more controls 50 on control panel 54c include the on/off state and/or the brightness level of these lights.

Control panel 54a includes a display 52 (FIG. 2) configured to display a plurality of different screens thereon. Surrounding display 52 are a plurality of navigation controls 50a-f that, when activated, cause the display 52 to display different screens on display 52. More specifically, when a user presses navigation control 50a, control panel 54a displays an exit detection control screen on display 52 that includes one or more icons that, when touched, control an onboard exit detection system 136 (FIG. 5). The exit detection system 136 is as adapted to issue an alert when a patient exits from patient support apparatus 20. Exit detection system 136 may include any of the same features and functions as, and/or may be constructed in any of the same manners as, the exit detection system disclosed in commonly assigned U.S. patent application 62/889,254 filed Aug. 20, 2019, by inventors Sujay Sukumaran et al. and entitled PERSON SUPPORT APPARATUS WITH ADJUSTABLE EXIT DETECTION ZONES, the complete disclosure of which is incorporated herein by reference.

In some embodiments, exit detection system 136 includes a plurality of load cells that support litter frame 28 at different locations and whose outputs are used to calculate a center of gravity of the patient. In such embodiments, if the patient's center of gravity moves outside of an active zone, system 136 issues an alert. An example of such a system is disclosed in commonly assigned U.S. Pat. No. 5,276,432 issued to Travis and entitled PATIENT EXIT DETECTION MECHANISM FOR HOSPITAL BED, the complete disclosure of which is hereby incorporated herein by reference. Other types of exit detection systems 136 may be included within patient support apparatus 20.

When a user presses navigation control 50b (FIG. 2), control panel 54 displays a monitoring control screen that includes a plurality of control icons that, when touched, control the onboard monitoring system 138 (FIG. 5) built into patient support apparatus 20. The onboard monitoring system 138 alerts the caregiver through a unified indicator, such as a light or a plurality of lights controlled in a unified manner, when any one or more of a plurality of settings on patient support apparatus 20 are in an undesired state, and uses that same unified indicator to indicate when all of the plurality of settings are in their respective desired states. Stated alternatively, monitoring system 138, when armed, monitors a plurality of conditions of patient support apparatus 20 (such as, but not limited to, any one or more of the following: brake status, siderail position, litter frame height, exit detection system 136, A/C cord status, nurse call cable status, etc.) and issues an alert if any one of those conditions are in an undesired state. Further details of one type of monitoring system that may be built into patient support apparatus 20 are disclosed in commonly assigned U.S. patent application Ser. No. 62/864,638 filed Jun. 21, 2019, by inventors Kurosh Nahavandi et al. and entitled PATIENT SUPPORT APPARATUS WITH CAREGIVER REMINDERS, as well as commonly assigned U.S. patent application Ser. No. 16/721,133 filed Dec. 19, 2019, by inventors Kurosh Nahavandi et al. and entitled PATIENT SUPPORT APPARATUSES WITH MOTION CUSTOMIZATION, the complete disclosures of both of which are incorporated herein by reference. Other types of monitoring systems may be included within patient support apparatus 20.

When a user presses navigation control 50c, control panel 54a displays a scale control screen that includes a plurality of control icons that, when touched, control a scale system 144 (FIG. 5) of patient support apparatus 20. Such a scale system 144 may include any of the same features and functions as, and/or may be constructed in any of the same manners as, the scale systems disclosed in commonly assigned U.S. patent application 62/889,254 filed Aug. 20, 2019, by inventors Sujay Sukumaran et al. and entitled PERSON SUPPORT APPARATUS WITH ADJUSTABLE EXIT DETECTION ZONES, and U.S. patent application Ser. No. 62/885,954 filed Aug. 13, 2019, by inventors Kurosh Nahavandi et al. and entitled PATIENT SUPPORT APPARATUS WITH EQUIPMENT WEIGHT LOG, the complete disclosures of both of which are incorporated herein by reference. The scale system may utilize the same force sensors (e.g. load cells) and/or other components that are utilized by the exit detection system 136, or it may utilize one or more different sensors and/or other components. Other scale systems besides those mentioned above in the '254 and '954 applications may alternatively be included within patient support apparatus 20.

When a user presses navigation control 50d, control panel 54 displays a motion control screen that includes a plurality of control icons that, when touched, control the movement of various components of patient support apparatus 20, such as, but not limited to, the height of litter frame 28 and the pivoting of head section 44. In some embodiments, the motion control screen displayed on display 52 in response to pressing control 50d may be the same as, or similar to, the position control screen 216 disclosed in commonly assigned U.S. patent application Ser. No. 62/885,953 filed Aug. 13, 2019, by inventors Kurosh Nahavandi et al. and entitled PATIENT SUPPORT APPARATUS WITH TOUCHSCREEN, the complete disclosure of which is incorporated herein by reference. Other types of motion control screens may be included on patient support apparatus 20.

When a user presses navigation control 50e, control panel 54a displays a motion lock control screen that includes a plurality of control icons that, when touched, control one or more motion lockout functions of patient support apparatus 20. Such motion lockout functions typically include the ability for a caregiver to use control panel 54a to lock out one or more of the motion controls 50 of the patient control panels 54c such that the patient is not able to use those controls 50 on control panels 54c to control the movement of one or more components of patient support apparatus 20. The motion lockout screen may include any of the features and functions as, and/or may be constructed in any of the same manners as, the motion lockout features, functions, and constructions disclosed in commonly assigned U.S. patent application Ser. No. 16/721,133 filed Dec. 19, 2019, by inventors Kurosh Nahavandi et al. and entitled PATIENT SUPPORT APPARATUSES WITH MOTION CUSTOMIZATION, the complete disclosure of which is incorporated herein by reference. Other types of motion lockouts may be included within patient support apparatus 20.

When a user presses on navigation control 50f, control panel 54a displays a menu screen that includes a plurality of menu icons that, when touched, bring up one or more additional screens for controlling and/or viewing one or more other aspects of patient support apparatus 20. Such other aspects include, but are not limited to, displaying information about one or more devices that are currently associated with patient support apparatus 20, diagnostic and/or service information for patient support apparatus 20, mattress control and/or status information, configuration settings, location information, and other settings and/or information. One example of such a menu screen is shown in commonly assigned U.S. patent application Ser. No. 62/885,953 filed Aug. 13, 2019, by inventors Kurosh Nahavandi et al. and entitled PATIENT SUPPORT APPARATUS WITH TOUCHSCREEN, the complete disclosure of which is incorporated herein by reference. Other types of menus and/or settings may be included within patient support apparatus 20. In at least one embodiment, utilization of navigation control 50f allows a user to navigate to a screen that enables a user to see which devices, if any, are currently associated with patient support apparatus 20. As will be discussed in greater detail below, patient support apparatus 20 includes an onboard locating system that is adapted to automatically determine the relative position of one or more devices with respect to patient support apparatus 20 and, in some instances, automatically associate and/or disassociate those devices with and/or from patient support apparatus 20 (and/or the patient assigned to patient support apparatus 20) depending upon the proximity of the device to patient support apparatus 20. Further details of this locating system are provided below.

For all of the navigation controls 50a-f (FIG. 2), screens other than the ones specifically mentioned above may be displayed on display 52 in other embodiments of patient support apparatus 20 in response to a user pressing these controls. Thus, it will be understood that the specific screens mentioned above are merely representative of the types of screens that are displayable on display 52 in response to a user pressing on one or more of navigation controls 50a-f. It will also be understood that, although navigation controls 50a-f have all been illustrated in the accompanying drawings as dedicated controls that are positioned adjacent display 52, any one or more of these controls 50a-f could alternatively be touchscreen controls that are displayed at one or more locations on display 52. Still further, although controls 50a-f have been shown herein as buttons, it will be understood that any of controls 50a-f could also, or alternatively, be switches, dials, or other types of non-button controls. Additionally, patient support apparatus 20 may be modified to include additional, fewer, and/or different navigation controls from the navigation controls 50a-f shown in FIG. 2.

FIG. 3 illustrates one example of a patient control panel 54c that may be incorporated into patient support apparatus 20 and positioned at a location on patient support apparatus 20 that is convenient for a patient to access while supported on support deck 30, such as on an interior side of one of the siderails 36. Control panel 54c includes a plurality of controls 50g-t that are intended to be operated by a patient. A nurse call control 50g, when pressed by the patient, sends a signal to a nurse call system requesting that a remotely positioned nurse talk to the patient. A Fowler-up control 50h, when pressed by the patient, causes a motorized actuator onboard patient support apparatus 20 to raise Fowler section 44 upwardly. A Fowler-down control 50i, when pressed by the patient, causes the motorized actuator to lower Fowler section 44 downwardly. A gatch-up control 50j, when pressed by the patient, causes another motorized actuator to raise a knee section of support deck 30, while a gatch-down control 50k causes the motorized actuator to lower the knee section of support deck 30. The knee section may refer to the joint that couples thigh section 46 to foot section 48.

A volume-up control 501, when pressed by the patient, causes patient support apparatus 20 to send a signal to an in-room television instructing it to increase its volume, while a volume down control 50m, when pressed, causes patient support apparatus 20 to send a signal to the television instructing it to decrease its volume. A channel-up control 50n, when pressed by the patient, causes patient support apparatus 20 to send a signal to the television instructing it to increase the channel number, while a channel-down control 500, when pressed, causes patient support apparatus 20 to send a signal to the television instructing it to decrease the channel number.

A mute control 50p, when pressed, causes patient support apparatus 20 to send a signal to the television instructing it to either mute itself or unmute itself, depending upon whether the television is currently muted or unmuted. In other words, mute control 50p is a toggle control that alternatingly sends mute and unmute commands to the television when it is pressed.

Power control 50q is a toggle control that, when pressed, sends a signal to the television to either turn on or turn off, depending upon the television's current power status. Closed-captioning control 50r is another toggle control that, when pressed, sends a signal to the television to either turn on its closed-captioning feature or to turn off its closed captioning feature, depending upon whether the closed-captioning feature is currently on or off.

Control 50s is a toggle control that, when pressed, sends a signal to a first light to either turn on or turn off, depending upon the current state of that first light. Control 50t is another toggle control that, when pressed, sends a signal to a second light to either turn on or turn off, depending upon the current state of that second light. In some embodiments, the first light is a reading light and the second light is a room light, both of which are positioned off-board the patient support apparatus 20.

It will be understood that not only the number of controls 50 on control panel 54c, but also the functions of the controls 50 on control panel 54c, the layout of the controls 50 on control panel 54c, and/or other aspects of control panel 54c may be modified from what is shown in FIG. 3. In some embodiments, control panel 54c is implemented on a pendant controller that includes a cable that is plugged into a port on patient support apparatus 20. In other embodiments, one or more of the controls 50 of control panel 54c may be omitted, augmented, and/or split amongst other controls panels and/or locations. Still other manners of implementing control panel 54c are also possible.

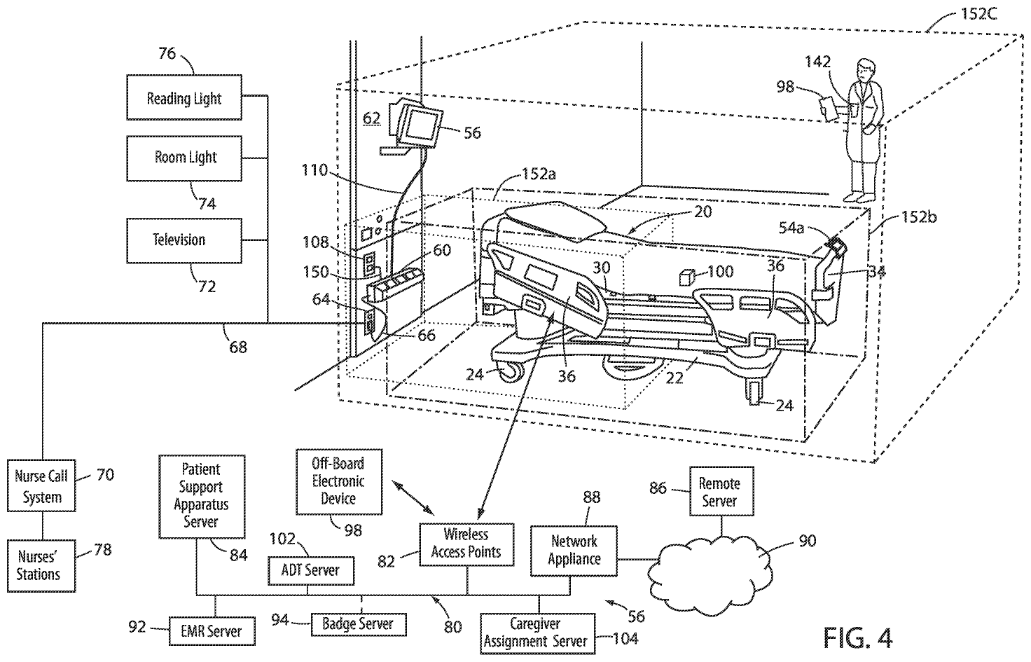

FIG. 4 illustrates patient support apparatus 20 positioned within a room 58 of a healthcare facility. FIG. 4 also illustrates additional items that may be present in a healthcare facility and which patient support apparatus 20 is configured to communicate with, including, but not limited to, a locator unit 60, a conventional local area network 80 of the healthcare facility, a caregiver badge 142, and one or more other devices 100 used during the care of a patient. Locator units 60 are positioned at known and fixed locations within the healthcare facility in which patient support apparatus 20 is positioned. Locator units 60 function as fixed locators. That is, locator units 60 communicate with patient support apparatuses 20 and share information with them that allows the location of the patient support apparatuses 20 to be determined.

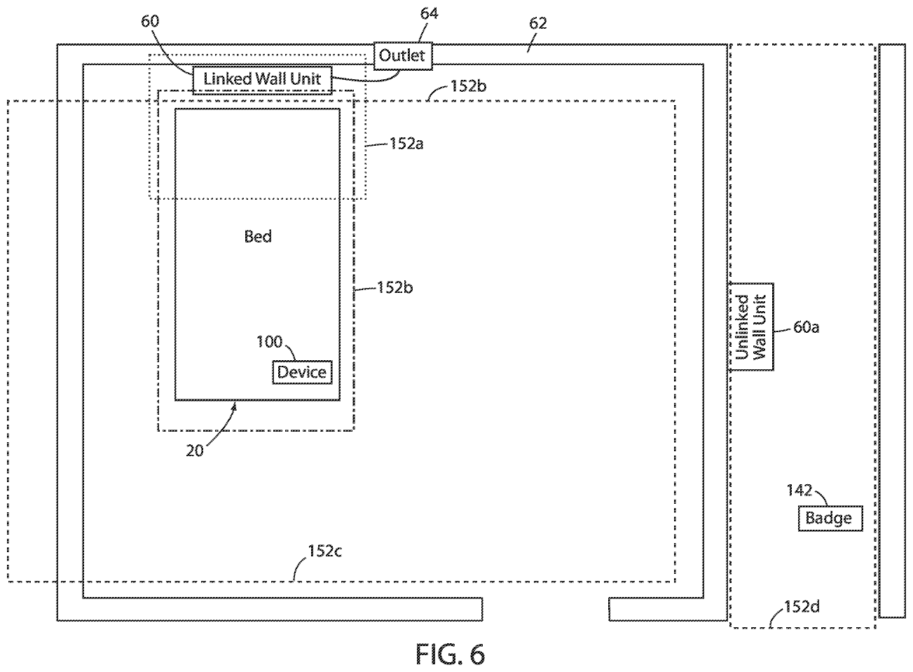

In some embodiments, patient support apparatus 20 is configured to be able to communicate with at least two different types of locator units 60: linked locator units 60 and unlinked locator units 60a. One example of a linked locator unit 60 is shown in FIG. 4. One example of an unlinked locator unit 60a is shown in FIG. 6. Other examples of unlinked locator units 60a are shown (and referred to as unlinked locator units 60b) in commonly assigned U.S. patent application Ser. No. 63/306,279 filed Feb. 3, 2022, by inventors Madhu Sandeep Thota et al. and entitled COMMUNICATION SYSTEM FOR PATIENT SUPPORT APPARATUSES, the complete disclosure of which is incorporated herein by reference. Patient support apparatus 20 is configured to communicate with the unlinked locator units 60b described in the aforementioned '279 application and to perform any one or more of the functions described therein that utilize such unlinked locator units and/or information provided by such unlinked locator units. Unless explicitly stated otherwise, all references herein to “locator units 60” without the term “linked” or “unlinked” in the reference will refer to both linked and unlinked locator units 60.

Linked locator units 60 are communicatively linked to a conventional communication outlet 64 and are adapted to provide location information to patient support apparatus 20. Linked locator units 60 are also adapted to serve as a communication conduit for routing wireless communications between patient support apparatus 20 and one or more devices and/or systems that are communicatively coupled to communication outlet 64 (e.g. room devices 72, 74, 76, and/or nurse call system 70, FIG. 4). In general, linked locator units 60 are typically positioned in patient rooms of the healthcare facility where one or more communication outlets 64 are typically present.

As shown in FIG. 4, linked locator unit 60 is adapted to be mounted to a wall 62, such as a headwall of a patient room 58 within the healthcare facility. The headwall of a conventional healthcare facility room 58 typically includes a conventional communications outlet 64 physically integrated therein. Communications outlet 64 is adapted to receive a nurse call cable 66 that physically connects at its other end either to patient support apparatus 20 (not shown) or to linked locator unit 60 (shown in FIG. 4). In many healthcare facilities, communication outlet 64 includes a 37-pin connector, although other types of connectors are often found in certain healthcare facilities. As will be discussed in greater detail below, linked locator unit 60 and nurse call cable 66 allow patient support apparatus 20 to communicate with a nurse call system, and one or more room devices positioned within room 58.

Communication outlet 64 is electrically coupled to one or more cables, wires, or other conductors 68 that electrically couple the communication outlet 64 to a nurse call system 70 and one or more conventional room devices, such as a television 72, a room light 74, and/or a reading light 76. Conductors 68 are typically located behind wall 62 and not visible. In some healthcare facilities, conductors 68 may first couple to a room interface circuit board that includes one or more conductors 68 for electrically coupling the room interface circuit board to room device 72, 74, 76 and/or nurse call system 70. Still other communicative arrangements for coupling communication outlet 64 to nurse call system 70 and/or one or more room devices 72, 74, 76 are possible.

Nurse call cable 66 (FIG. 4) enables linked locator unit 60 to communicate with nurse call system 70 and/or room devices 72, 74, 76. Because patient support apparatus 20 is able to wirelessly communicate with linked locator unit 60, patient support apparatus 20 is thereby able to communicate with nurse call system 70 and room devices 72, 74, 76. A patient supported on patient support apparatus 20 who activates a nurse call control (e.g. 50g; see FIG. 3) on patient support apparatus 20 causes a signal to be wirelessly sent from patient support apparatus 20 to linked locator unit 60, which in turn conveys the signal via nurse call cable 66 to the nurse call system 70, which forwards the signal to one or more remotely located nurses (e.g. nurses at one or more nurse's stations 78). If the patient activates one or more room device controls (e.g. controls 50l-t; see FIG. 3), one or more wireless signals are conveyed to linked locator unit 60, which in turn sends appropriate signals via nurse call cable 66 to communication outlet 64 and the room device 72, 74, 76 that change one or more features of these devices (e.g. the volume, channel, on/off state, etc.).

As is also shown in FIG. 4, patient support apparatus 20 is further configured to communicate with a local area network 80 of the healthcare facility. In the embodiment shown in FIG. 4, patient support apparatus 20 includes a wireless network transceiver 96 (FIG. 5) that communicates wirelessly with local area network 80. Network transceiver 96 is, in at least some embodiments, a WiFi transceiver (e.g. IEEE 802.11) that wirelessly communicates with one or more conventional wireless access points 82 of local area network 80. In other embodiments, network transceiver 96 may be a wireless transceiver that uses conventional 5G technology to communicate with network 80, one or more servers hosted thereon, and/or other devices. In some embodiments, network transceiver 96 may include any of the structures and/or functionality of the communication modules 56 disclosed in commonly assigned U.S. Pat. No. 10,500,401 issued to Michael Hayes and entitled NETWORK COMMUNICATION FOR PATIENT SUPPORT APPARATUSES, the complete disclosure of which is incorporated herein by reference. Still other types of wireless network transceivers may be utilized.

In some embodiments, network transceiver 96 is a wired transceiver that is adapted to allow patient support apparatus 20 to communicate with network 80 via a wired connection, such as an Ethernet cable that plugs into an Ethernet port (e.g. an RJ-45 style port, an 8P8C port, etc.) built into patient support apparatus 20. In still other embodiments, patient support apparatus 20 includes both a wired transceiver 96 for communicating with network 80 via a wired connection and a wireless transceiver 96 for wirelessly communicating with network 80.

Patient support apparatus 20 is configured to communicate with one or more servers on local area network 80 of the healthcare facility. One such server is a patient support apparatus server 84. Patient support apparatus server 84 is adapted, in at least one embodiment, to receive data from the patient support apparatuses 20 positioned within the healthcare facility and distribute this data to caregivers, other servers, and/or other software applications. As will be discussed in greater detail below, server 84 may also be configured to receive data from one or more devices 100 and/or badges 142 that are positioned within one or more volumes of space defined around patient support apparatus 20 and/or within a volume of space defined around locator units 60. Such information may be forwarded from the device(s) to patient support apparatus 20, and from patient support apparatus 20 to server 84 via network transceiver 96 and one or more access points 82. In some embodiments where data from medical devices is collected, the data from one or more of the medical devices may be forwarded to an Electronic Medical Records (EMR) server 92, and/or to one or more other servers on network 80 (and/or one or more electronic devices 98), such as a caregiver assistance server and/or a caregiver assistance software application, as will also be discussed in greater detail below.

In some embodiments, patient support apparatus server 84 is configured to communicate at least some of the patient support apparatus data and/or device data received from patient support apparatuses 20 to a remote server 86 that is positioned geographically remotely from the healthcare facility. Such communication may take place via a conventional network appliance 88, such as, but not limited to, a router and/or a gateway, that is coupled to the Internet 90. The remote server 86, in turn, is also coupled to the Internet 90, and patient support apparatus server 84 is provided with the URL and/or other information necessary to communicate with remote server 86 via the Internet connection between network 80 and server 86.

In some alternative embodiments, patient support apparatus 20 may be configured to communicate directly with one or more cloud-based servers, such as remote server 86, without utilizing patient support apparatus server 84. That is, in some embodiments, patient support apparatuses 20 may be configured to communicate directly with a remote server without relying upon any locally hosted servers (e.g. servers hosted on network 80). Patient support apparatus 20 is provided with the URL and/or other information necessary to communicate with remote server 86 via the Internet connection between network 80 and remote server 86. In some such embodiments, network appliance 88 is a router configured to support such direct connections. Still other types of direct-to-cloud connections may be utilized with one or more of patient support apparatuses 20. When patient support apparatus 20 is configured to directly communicate with remote server 86, patient support apparatus server 84 may be omitted and any one or more of the functions of patient support apparatus server 84 described herein may be performed by remote server 86.

Patient support apparatus server 84 is also configured to determine the location of each patient support apparatus 20 (and/or its associated devices), or receive the location of each patient support apparatus 20 (and/or its associated devices) from the patient support apparatuses 20 themselves. In some embodiments, patient support apparatus server 84 determines the room number and/or bay area of each patient support apparatus 20 and its associated devices that are positioned within a room 58, as well as the location of patient support apparatuses 20 and their associated devices that are positioned outside of a room 58, such as those that may be positioned in a hallway, a maintenance area, or some other area. In general, patient support apparatus server 84 may be configured to determine the position of any patient support apparatus 20 that is positioned within communication range of one or more locator units 60, as well as the location of any associated devices that are positioned within one or more volumes of space defined around the patient support apparatus 20, as will be discussed in greater detail below.

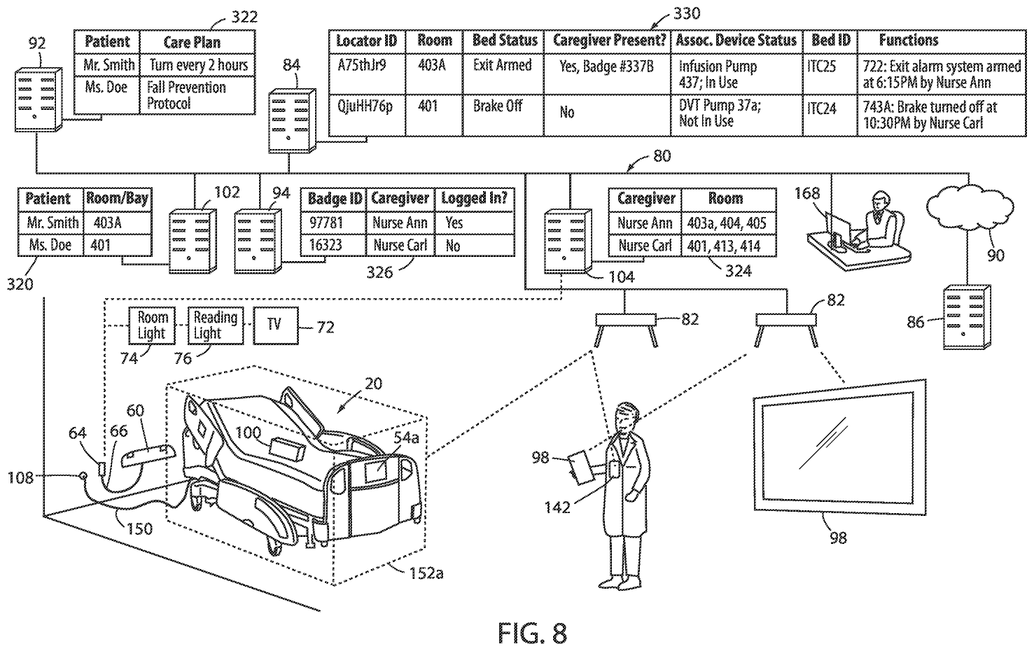

Patient support apparatus server 84 (FIGS. 4 & 8) is adapted to communicate with a plurality of other servers, such as a conventional EMR server 92, a conventional badge server 94, a conventional Admission, Discharge, and Transfer (ADT) server 102, and/or a conventional caregiver assignment server 104. EMR server 92 stores individual patient records. Such patient records identify a patient by name and include medical information associated with that patient. Such medical information may include all of the medical information generated from the patient's current stay in the healthcare facility as well as medical information from previous visits. EMR server 92 stores data such as that shown in table 322 (FIG. 8). Table 322 shows an abbreviated example of several types of medical information entries that are commonly found within a patient's medical records: a fall risk entry indicating whether the patient is a fall risk, a bed sore risk entry indicating whether the patient is at risk for developing bed sores, and a care plan for a particular patient. EMR server 92 includes far more additional information in the medical records of each patient than what is shown in table 322. It will be understood that the term “EMR server,” as used herein, also includes Electronic Health Records servers, or EHR servers for short, and that the present disclosure does not distinguish between electronic medical records and electronic health records.

Caregiver assignment server 104 (FIGS. 4 and 8) includes a caregiver assignment table 324 that matches caregivers to specific rooms and/or bays within the healthcare facility. Caregiver assignment server 104 stores information regarding shift changes, personnel, and the general assignments of caregivers who are employed by the healthcare facility. Although table 324 only shows two caregivers who are each assigned to three rooms, it will be understood that more than two caregivers will typically be present in a healthcare facility and they may have lessor or greater numbers of room assignments. In some caregiver assignment servers 104, caregivers are assigned to specific patients, rather than to specific rooms, in which case table 324 may correlate caregivers to individual patients rather than rooms. Still further, some conventional nurse call systems may be configured to carry out the functions of caregiver assignment server 104, in which case caregiver assignment server 104 may be replaced by and/or supplemented with a nurse call server. FIG. 8 shows an example of this where caregiver assignment server 104 is coupled by a dotted line to communication outlet 64. In this particular example, caregiver assignment server 104 also functions as a nurse call server that oversees the communications between patients and their caregivers.

ADT server 102 stores patient information, including the identity of patients and the corresponding rooms 58 and/or bays within rooms to which the patients are assigned. That is, ADT server 102 includes a patient-room assignment table 320 (FIG. 8). The patient-room assignment table 320 correlates rooms, as well as bays within multi-patient rooms, to the names of individual patients within the healthcare facility. The patient's names are entered into the ADT server 102 by one or more healthcare workers whenever a patient checks into the healthcare facility and the patient is assigned to a particular room within the healthcare facility. If and/or when a patient is transferred to a different room and/or discharged from the healthcare facility, the staff of the healthcare facility update ADT server 102. ADT server 102 therefore maintains an up-to-date table 320 that correlates patient names with their assigned rooms and/or bays. In some conventional electronic medical record systems, the functions of the ADT server 102 may be incorporated into the EMR system, and EMR server 92 may therefore, in some embodiments, carry out the functions of ADT server 102.

Badge server 94 (FIGS. 4 & 8) is configured to manage communications between, and keep track of the location of, badges 142 that are typically worn by healthcare workers, such as caregivers, service technicians, cleaning personnel, transportation assistants, etc. Badge server 94 typically maintains a table 326 (FIG. 8) that correlates badge IDs with individual healthcare workers. Each badge 142 includes a unique ID 166 (FIG. 5) that distinguishes that badge 142 from other badges 142. When a healthcare provider arrives at a healthcare facility, he or she typically grabs a badge 142 from a common collection of badges 142 and wears it for the duration of his or her work shift (and returns it to the general collection after his/her shift). In conventional badge servers 94, in order for server 94 to know that a particular badge 142 is assigned to a particular healthcare worker, the worker has to manually associate the particular badge 142 they chose to wear that day with their name (or some other worker ID). This manual association may involve scanning the badge on a badge reader in communication with server 94, typing information (such as the badge ID and/or the worker's ID) into a computer coupled to server 94, entering information into badge 142 (if it is a badge that allows data entry), performing other actions, and/or a combination of these steps.

Badge server 94 may also be configured to monitor the location of badges 142 within a healthcare facility. Typically this location monitoring is performed through the monitoring of the wireless access points 82 throughout the healthcare facility. That is, badges 142 are often equipped to use WiFi, or other wireless communication protocols, that allow them to communicate with wireless access points 82. By monitoring which access points the badges 142 are currently connected to, using a map of the location of the access point 82 within the facility, and, in some cases, using the signal strengths between the badges 142 and one or more of the access points 82, the general position of the badges 142 within the healthcare facility can be determined by badge server 94. Often this location determination is low resolution. That is, for example, this location-determining technique may only indicate which room the caregiver's badge 142 is currently located in, or which hallway, but might not provide accurate information as to where the badge 142 is located within a particular room or hallway. As will be discussed in greater detail below, patient support apparatuses 20 are equipped with a separate and independent location-determination technology that can be used to monitor the location of badges 142 (and/or other devices) with greater accuracy, such as locations that are accurate to within five to ten centimeters (although other accuracy levels may be utilized in accordance with this disclosure).

Badges 142 may be badges of the type sold or marketed by Stryker Corporation of Kalamazoo, Michigan, under the names Vocera Badge, Vocera Smartbadge, and/or Vocera Minibadge. Other types of badges may also, or alternatively, be used. Such badges 142 include the ability to transmit voice communications of healthcare workers to other badges 142 and/or other locations within a healthcare facility. Some of the badges may also include text messaging abilities, alarm notifications, and other functions. When integrated into the system described herein, such badges 142 may be modified to include one or more ultra-wideband transceivers and/or tags that communicate with ultra-wideband transceivers onboard patient support apparatus 20, as will be discussed in greater detail herein. That is, patient support apparatus 20 may be configured to repetitively determine the location of any of the badges 142 that are positioned within range of its ultra-wideband transceivers and determine whether the badge 142 is positioned inside or outside of one or more volumes of space, as will also be discussed in greater detail below. In some embodiments, badges 142 that do not include UWB transceivers may also be incorporated into the communication system described herein

In some embodiments, patient support apparatus server 84 (FIGS. 4 & 8) communicates with EMR server 92 in order to transmit patient data that is to be recorded in a patient's health record (e.g. vital sign readings from one or more vital sign sensors; weight readings taken from the scales built into patient support apparatuses 20; therapies provided to patients using a powered mattress 42 onboard patient support apparatuses 20; data from other devices that are determined to be associated with the patient assigned to patient support apparatus 20, etc.). In addition, server 84 communicates with EMR server 92, in some embodiments, in order to receive data from one or more of the devices that are being used with a particular patient.

It will be understood that the architecture and content of local area network 80 will vary from healthcare facility to healthcare facility, and that the examples shown in FIGS. 4 & 8 are merely two examples of the type of network a healthcare facility may be employ. Typically, one or more additional servers will be hosted on network 80 and one or more of them may be adapted to communicate with patient support apparatus server 84. For example, some healthcare facilities may include a server that oversees the coordination and communication involved with transporting patients to different locations within the healthcare facility, including both intra-facility transportation and transportation to one or more exits (such as when a patient is discharged from the facility). As another example, some healthcare facilities may include a server that oversees that monitoring of which patient support apparatuses 20 have been cleaned, which ones need to be cleaned, and/or which ones are in the process of being cleaned. Still other types of servers may be utilized in some healthcare facilities. It will be understood that, in accordance with principles disclosed herein, some of the functions of these other servers (e.g. transportation management and/or cleaning management) may be integrated into one or more of the servers shown in FIG. 4 (e.g. patient support apparatus server 84 may be configured to perform one or more of these functions, in some embodiments), and/or patient support apparatus server 84 may be configured to communicate with the servers that perform these functions.

In addition to the aforementioned server, local area network 80 will also typically allow one or more electronic devices 98 to access the local area network 80 and the servers hosted thereon via wireless access points 82. Such electronic devices 98 include, but are not limited to, smart phones, tablet computers, portable laptops, desktop computers, smart televisions, and other types of electronic devices that include a WiFi capability and that are provided with the proper credentials (e.g. SSID, password, etc.) to access network 80 (and, in at least some situations, patient support apparatus server 84). As will be discussed in further detail herein, patient support apparatus server 84 is configured, in some embodiments, to share data with one or more electronic devices 98 that relates to patient support apparatus 20, that relates to one or more devices 100 that become associated with patient support apparatus 20 (or the patient assigned thereto), that relates to one or more badges 142 that become associated with patient support apparatus 20, and/or that relates to one or more medical records of the patient stored in EMR server 92.

Linked locator units 60 are adapted to wirelessly receive signals from patient support apparatus 20 and deliver the signals to communications outlet 64 in a manner that matches the way the signals would otherwise be delivered to communications outlet 64 if a conventional nurse call cable 66 were connected directly between patient support apparatus 20 (via a cable port 148; FIG. 5) and communications outlet 64. Linked locator units 60 are also adapted to transmit signals received from communications outlet 64 to patient support apparatus 20 via a BT transceiver 106 and/or a UWB transceiver 126 (FIG. 5). Thus, patient support apparatus 20 and linked locator unit 60 cooperate to send signals to, and receive signals from, communications outlet 64 in a manner that is transparent to communications outlet 64 such that outlet 64 cannot detect whether it is in communication with patient support apparatus 20 via a wired connection or it is in communication with patient support apparatus 20 via a wireless connection between patient support apparatus 20 and linked locator unit 60 (the latter of which is in wired communication with outlet 64). In this manner, a healthcare facility can utilize the wireless communication abilities of one or more patient support apparatuses 20 without having to make any changes to their existing communication outlets 64.

As noted, in addition to sending signals received from patient support apparatus 20 to communications outlet 64, linked locator units 60 are also adapted to forward signals received from communications outlet 64 to patient support apparatus 20. Linked locator units 60 are therefore adapted to provide bidirectional communication between patient support apparatus 20 and communications outlet 64. This bidirectional communication includes, but is not limited to, communicating command signals from any of controls 50 and/or from any of electronic devices 98 to corresponding room devices 72, 74, and/or 76 and communicating audio signals between a person supported on patient support apparatus 20 and a caregiver positioned remotely from patient support apparatus 20. The audio signals received by linked locator unit 60 from a microphone on patient support apparatus 20 are forwarded to communications outlet 64 (for forwarding to nurse call system 70), and the audio signals of a remotely positioned nurse that are received at communications outlet 64 (from nurse call system 70) are forwarded to a speaker onboard patient support apparatus 20.

Nurse call cable 66, in some embodiments, includes a conventional 37 pin connector on each end, one of which is adapted to be inserted into outlet 64 and the other one of which is adapted to be inserted into a linked locator unit 60 (or cable port 148 of patient support apparatus 20 if wired communication is desired). Such 37 pin connections are one of the most common types of connectors found on existing walls of medical facilities for making connections to the nurse call system 70 and room devices 72, 74, and 76. Linked locator unit 60 and nurse call cable 66 are therefore configured to mate with one of the most common type of communication outlets 64 used in medical facilities. Such 37 pin connectors, however, are not the only type of connectors, and it will be understood that linked locator units 60 can utilize different types of connectors that are adapted to electrically couple to different types of nurse call cables 66 and/or different types of communication outlets 64. One example of such an alternative communications outlet 64 and cable 66 is disclosed in commonly assigned U.S. patent application Ser. No. 14/819,844 filed Aug. 6, 2015 by inventors Krishna Bhimavarapu et al. and entitled PATIENT SUPPORT APPARATUSES WITH WIRELESS HEADWALL COMMUNICATION, the complete disclosure of which is incorporated herein by reference. Still other types of communication outlets 64 and corresponding connectors may be utilized.

Locator unit 60 (FIG. 4) also includes an electrical cord 150 having a plug positioned at a far end that is adapted to be inserted into a conventional electrical outlet 108. Electrical cord 150 enables locator unit 60 to receive power from the mains electrical supply via outlet 108. It will be appreciated that, in some embodiments, locator unit 60 is battery operated and cord 150 may be omitted. In still other embodiments, locator unit 60 may be both battery operated and include cord 150 so that in the event of a power failure, battery power supplies power to locator unit 60, and/or in the event of a battery failure, electrical power is received through outlet 108.

In some embodiments, locator units 60 include a video port 120 that is adapted to receive a display cable 110 (FIG. 4). The display cable 110 is adapted to couple to locator unit 60 at one end and a display device 56 at its opposite send. Locator unit 60 may be configured to use cable 110 to send data to display device 56 that is to be displayed thereon. Such data may include data from one or more devices 100 that are associated with the patient on patient support apparatus 20 (or with patient support apparatus 20 itself), data from one or more badges 142, data from one or more sensors onboard patient support apparatus 20, location data regarding the location of patient support apparatus 20, and/or other data. Cable 110 may be a High-Definition Multimedia Interface (HDMI) cable, a Video Graphics Array (VGA) cable, a DisplayPort (DP) cable, a plurality of Radio Corporation of America (RCA) cables, a Digital Visual Interface (DVI) cable, and/or another type of cable. Locator unit 60 is configured to include a complementary type of connector that mates with a connector on an end of cable 110. Further details regarding the communication between patient support apparatus 20 and display device 56 are provided below and, in some embodiments, patient support apparatus 20 may be configured to communicate directly with certain display devices without using locator unit 60 as a communication intermediary.

In addition to the other functions described herein, locator units 60 are configured to communicate location data to patient support apparatus 20 that enables patient support apparatus 20 and/or patient support apparatus server 84 to determine the location of patient support apparatus 20 within the healthcare facility. In general, such location determination is carried out by patient support apparatus 20 analyzing wireless signals communicated between itself and locator unit 60 in order to determine its position relative to locator unit 60. If patient support apparatus 20, or a predefined reference point on patient support apparatus 20 (e.g. its head end, its center, etc.) is positioned within a threshold distance of locator unit 60, patient support apparatus 20 associates itself with the locator unit 60. When associated, patient support apparatus 20 may communicate data to locator unit 60, receive data from locator unit 60, and also deem its location within the healthcare facility to be the same as location of locator unit 60. When patient support apparatus 20 is outside of the threshold distance, it does not associate itself with locator unit 60, and therefore does not exchange data with locator unit 60 or consider its location to be the same as that of locator unit 60's location.

In some embodiments, patient support apparatus 20 is configured to associate itself with a particular locator unit 60 if controller 140 determines that the locator unit 60 is within a volume of space 152a (FIGS. 4 & 6), or locator unit 60 determines that patient support apparatus 20 (or a reference point thereon) is positioned within volume of space 152a. In some embodiments, the volume of space 152a is defined with respect to each locator unit 60 and does not move. In other embodiments, the volume of space 152a is defined with respect to patient support apparatus 20 and moves as patient support apparatus 20 moves. In some embodiments, patient support apparatus 20 associates itself with a nearby locator unit 60 if both the locator unit 60 and the patient support apparatus 20 (or a reference point thereon) are concurrently within the predefined volume of space 152a. Regardless of whether volume of space 152a is defined with respect to a locator unit 60, or with respect to a patient support apparatus 20, by at least one or both of these devices (locator unit 60 and patient support apparatus 20) being positioned within the predefined volume of space 152a, the locator unit 60 and patient support apparatus 20 will be positioned within a threshold distance of each other. An example of patient support apparatus 20 occupying a volume of space 152a is shown in FIG. 4, where head end 38 of patient support apparatus 20 is positioned inside of volume of space 152a (and thus within a predefined distance of locator unit 60), and patient support apparatus 20 has therefore associated itself with that particular locator unit 60.

After associating itself with a particular locator unit 60, patient support apparatus 20 is configured to be able to have its absolute position within the healthcare facility determined by receiving a unique locator identifier (ID) 122 (FIG. 5) from the locator unit 60. The location of each locator unit 60 in the healthcare facility is surveyed during the installation of locator units 60, and the unique IDs 122 of each locator unit 60 are also recorded during the installation of locator units 60. This surveying information and corresponding ID information may be stored in patient support apparatus server 84 and/or onboard the patient support apparatuses 20, thereby enabling a patient support apparatus 20 and/or patient support apparatus server 84 to determine the location of a patient support apparatus 20 once it is associated with a particular locator unit 60.

In those embodiments where patient support apparatus server 84 is configured to determine the location of patient support apparatus 20, patient support apparatus 20 sends its relative position information with respect to the associated locator unit 60, and/or the ID 122 of the associated locator unit 60 (and its own unique patient support apparatus ID 130 (FIG. 5)) to server 84. Server 84 includes a table of all of the locations of the locator units 60 (which, as noted, is generated via a surveying operation during the installation of locator units 60), and it uses that table to correlate the patient support apparatus IDs 130 and the locator unit IDs 122 it receives to specific locations within the healthcare facility. Thus, if a particular patient support apparatus 20 (with a particular ID 130) sends to server 84 an associated locator unit ID 122 that corresponds to room 430, server 84 determines that that particular patient support apparatus 20 is currently located in room 430. Generally speaking, and as will be discussed in greater detail below, the location of a patient support apparatus 20 is deemed to correspond to whichever locator unit 60 it is currently associated with, and if it is not currently associated with any locator unit 60, its location may be considered to be indeterminate.

In some embodiments of patient support apparatuses 20 and locator units 60, the relative location of a patient support apparatus 20 to a locator unit 60 is determined solely using ultra-wideband communication between the patient support apparatus 20 and the locator unit 60. Alternatively, in some embodiments, patient support apparatus 20 solely uses short range infrared communications with locator unit 60 to determine its relative location, wherein such short range infrared communications are only possible when the patient support apparatus 20 is positioned within a close proximity to the locator unit 60 (e.g. in the range of about 1-3 unobstructed meters). In these latter embodiments, patient support apparatus 20 may report that its location coincides with that of the nearby locator unit 60 when it is able to successfully communicate with the nearby locator unit 60 using these short range infrared communications. Still further, in some embodiments, patient support apparatus 20 and locator unit 60 may communicate with each other using both infrared and ultra-wideband communications. Further details regarding the use of short range infrared communications for location determination are described in commonly assigned U.S. Pat. No. 9,999,375 issued Jun. 19, 2018, to inventors Michael Hayes et al. and entitled LOCATION DETECTION SYSTEMS AND METHODS, the complete disclosure of which is incorporated herein by reference.

In some embodiments, locator units 60 and/or patient support apparatuses 20 may be constructed to include any or all of the functionality of the wireless headwall units and/or patient support apparatuses disclosed in commonly assigned U.S. patent application Ser. No. 14/819,844 filed Aug. 6, 2015, by inventors Krishna Bhimavarapu et al. and entitled PATIENT SUPPORT APPARATUSES WITH WIRELESS HEADWALL COMMUNICATION; in commonly assigned U.S. patent application Ser. No. 63/26,937 filed May 19, 2020, by inventors Alexander Bodurka et al. and entitled PATIENT SUPPORT APPARATUSES WITH HEADWALL COMMUNICATION; and/or in commonly assigned U.S. patent application Ser. No. 63/245,245 filed Sep. 17, 2021, by inventors Kirby Neihouser et al. and entitled SYSTEM FOR LOCATING PATIENT SUPPORT APPARATUSES, the complete disclosures of all of which are incorporated herein by reference.

Still further, in some embodiments, locator units 60 and/or patient support apparatuses 20 may be constructed to include any of the features and/or functions of the headwall units 144a and/or patient support apparatuses disclosed in commonly assigned U.S. patent application Ser. No. 63/131,508 filed Dec. 29, 2020, by inventors Kirby Neihouser et al. and entitled TOOL FOR CONFIGURING HEADWALL UNITS USED FOR PATIENT SUPPORT APPARATUS COMMUNICATION, the complete disclosure of which is incorporated herein by reference.

FIG. 5 depicts a block diagram of patient support apparatus 20, a linked locator unit 60, a device 100, a badge 142, a display device 56, and network 80. As will be discussed in greater detail below, patient support apparatus 20 is configured to automatically determine the location of one or more locator units 60, badges 142, devices 100 that have built-in UWB transceivers, or objects 190 that have a UWB tag 188 attached to them. In addition, patient support apparatus 20 is configured to automatically carry out communications with these objects (locator unit 60, device 100, badges 142, and/or tag 188) if they are positioned within a defined proximity to patient support apparatus 20. In some embodiments, if a particular object is positioned within the defined proximity, patient support apparatus 20 automatically associates the object with patient support apparatus 20 (and/or the patient assigned to patient support apparatus 20), and causes data from that object (or objects) to be displayed on one or more of display devices 56, and/or forwards data from that object (or objects) to patient support apparatus server 84, EMR server 92, and/or another server in communication with network 80. When the object is positioned outside the defined proximity, patient support apparatus 20 may automatically disassociate itself from the object and, in some situations, terminate communications with the object and/or inform patient support apparatus server 84 of the disassociation.

Linked locator unit 60 (FIG. 5) includes an ultra-wideband transceiver 126, a Bluetooth transceiver 106, a locator unit controller 112, configuration circuitry 114, a television controller 116, a headwall interface 118, a video port 120, a unit ID 122, and, in some embodiments, an infrared transceiver 124. Bluetooth transceiver 106 is adapted to communicate with a Bluetooth transceiver 128 onboard patient support apparatus 20 using RF waves in accordance with conventional Bluetooth standards (e.g. IEEE 802.14.1 and/or any of the standards maintained by the Bluetooth Special Interest Group (SIG) of Kirkland, Washington, USA). In some embodiments, transceivers 106 and 128 utilize Bluetooth Low Energy communications.

Ultra-wideband transceiver 126 is adapted to communicate with one or more ultra-wideband transceivers 132 positioned onboard patient support apparatus 20. Transceiver 126 is adapted to determine a distance between itself and patient support apparatus 20. Alternatively, or additionally, transceiver 126 may be adapted to allow one or more of the UWB transceivers 132 onboard patient support apparatus 20 to determine their distance(s) from transceiver 126. In some embodiments, transceivers 126 and 132 use time of flight (TOF) computations to determine these distances. In other embodiments, transceivers 126 and 132 may utilize other techniques (e.g. time difference of arrival, two-way ranging, angle of arrival, channel state information, etc.) for determining their distances from each other, either in addition to, or in lieu of, TOF computations. In some embodiments, transceivers 126, 132 may also determine an angle between themselves using angular information derived from antenna arrays positions onboard transceivers 126, 132, or by using other techniques. The position and orientation of each transceiver 132 onboard patient support apparatus 20 is known and stored in an onboard memory 134 and used to determine the position and orientation of patient support apparatus 20 with respect to the locator unit(s) 60 with which it is communicating. Such position and orientation information may be determined using conventional trilateration and/or triangulation techniques, or other techniques.

In some embodiments, transceivers 126, 132 are implemented as any of the Trimension™ ultra-wideband modules available from NXP Semiconductors of Austin, Texas. These modules include, but are not limited to, the Trimension™ UWB modules ASMOP1BOON1, ASMOP1CO0R1, and/or the ASMOP1CO0A1, that utilize any of the following chips: the NXP SR150, SR100T, SR040, NCJ29D5, and/or the OL23DO chips. Modules manufactured and/or marketed by other companies may also be used, including, but not limited to, the Decawave DWM1000, DWM10001C, DWM3000 modules (available from Decawave of Dublin, Ireland); the Nordic TSG5162 SiP module (available from Tsingoal Technology of Beijing, China); and/or the UWB hub, wand, and/or sensors available from Zebra technologies of Lincolnshire, Illinois. Still other types of UWB modules may be used to implement transceivers 126 and 132.

Locator unit controller 112 is adapted to control the operation of transceivers 126, 106, configuration circuitry 114, TV controller 116, headwall interface 118, video port 120, and, if included, IR transceiver 124 (FIG. 5). When infrared transceiver 124 is included, it may be included to provide backwards compatibility to patient support apparatuses 20 that are not equipped with a UWB transceiver 132. That is, some healthcare facilities may include one or more patient support apparatuses that are not equipped with a UWB transceiver 132, but that do include an IR transceiver that is adapted to communicate with IR transceiver 124. When locator unit 60 includes IR transceiver 124, it is able to communicate its unit ID 122 to such patient support apparatuses via IR transceiver 124, which is a short range transceiver that is configured to only communicate with an adjacent patient support apparatus when the patient support apparatus is nearby (e.g. without about five feet or so). Such an adjacent patient support apparatus 20 then communicates the received locator unit ID 122 along with its own unique ID 130 (FIG. 5) to server 84 which, as noted previously, is able to correlate the locator unit ID 122 to a particular location with the healthcare facility. In this manner, server 84 is able to use locator units 60 determine the location of versions of patient support apparatuses 20 that don't have a UWB transceiver 132, but that do have an IR transceiver.

Headwall interface 118 is adapted to change the electrical state of one or more pins that are in electrical communication with communication outlet 64 (via cable 66). Headwall interface 118 changes these electrical states in response to instructions from controller 112. For example, if the exit detection system 136 of patient support apparatus 20 detects a patient exit, a controller 140 of patient support apparatus 20 sends an exit alert signal to linked locator unit 60 and controller 112 responds by instructing headwall interface 118 to change the electrical state of at least one pin that is used to signal an exit alert (or a generic priority alert) to the nurse call system 70 via communications outlet 64. Additionally, if a device 100, such as a portable exit detection sensor, is associated with patient support apparatus 20 and it detects a patient exit, the exit detection sensor may transmit an exit detection alert signal to patient support apparatus 20, which in turn forwards the exit alert signal to linked locator unit 60, and controller 112 responds by instructing headwall interface 118 to change the electrical state of the same pin or pins that it does in response to receiving an exit detection alert from exit detection system 136.

In some embodiments, headwall interface 118 may be constructed in the same manner as, and/or may include any one or of the functions as, the cable interface 88 described in commonly assigned U.S. patent application Ser. No. 63/193,778 filed May 27, 2021, by inventors Krishna Bhimavarapu et al. and entitled PATIENT SUPPORT APPARATUS AND HEADWALL UNIT SYNCING, the complete disclosure of which is incorporated herein by reference. Alternatively, or additionally, headwall interface 118 may be constructed in the same manner as, and/or may include any one or more of the same functions as, the headwall interface 120 disclosed in commonly assigned U.S. patent application Ser. No. 63/131,508 filed Dec. 29, 2020, by inventors Kirby Neihouser et al. and entitled TOOL FOR CONFIGURING HEADWALL UNITS USED FOR PATIENT SUPPORT APPARATUS COMMUNICATION, the complete disclosure of which is incorporated herein by reference. Linked locator unit 60 may also be configured to perform any of the functions of the headwall units 94 disclosed in the above-mentioned '778 patent application.

Configuration circuitry 114 and TV controller 116 may be configured to perform any of the same functions as, and/or be constructed in any of the same manners as, the configuration circuitry 132 and the TV control circuit 134, respectively, of commonly assigned U.S. patent application Ser. No. 63/131,508 filed Dec. 29, 2020, by inventors Kirby Neihouser et al. and entitled TOOL FOR CONFIGURING HEADWALL UNITS USED FOR PATIENT SUPPORT APPARATUS COMMUNICATION, the complete disclosure of which has already been incorporated herein by reference. Additionally, or alternatively, linked locator unit 60 may be configured to perform any of the functions of the headwall units 144 disclosed in the aforementioned '508 patent application.

Patient support apparatus 20 includes a controller 140, a memory 134, exit detection system 136, a scale system 144, monitoring system 138, a microphone 146, Bluetooth transceiver 128, one or more UWB transceivers 132, display 52 (which may be part of control panel 54a, and/or another control panel 54), network transceiver 96, a nurse call interface 154, brake 250, an obstruction detection system 252, and a plurality of additional components that are not shown in FIG. 5. Patient support apparatus 20 may also include a plurality of additional components that are not shown in FIG. 5. In other embodiments, patient support apparatus 20 may include different a different combination of some or all of the components shown in FIG. 5 and/or other components not shown therein. In some embodiments, patient support apparatus 20 may omit one or more of the components shown in FIG. 5.

Each UWB transceiver 132 is positioned at a known location on patient support apparatus 20. This known location information is stored in memory 134 and/or elsewhere, and may be defined with respect to any suitable frame of reference that is common to patient support apparatus 20. The known location information may include the spatial relationship between UWB transceivers 132 and/or any other components of patient support apparatus 20. For example, in some embodiments, the known location information includes the spatial relationship not only between UWB transceivers 132, but also the spatial relationships between UWB transceivers 132 and one or more of the following: the head end 38 of patient support apparatus 20, the foot end 40 of patient support apparatus 20, the sides of patient support apparatus 20, a reference point defined on patient support apparatus 20, the floor, and/or other components and/or landmarks of patient support apparatus 20. In some embodiments, this location information is used to determine the orientation of patient support apparatus 20 with respect to one or more walls 62, locator units 60, another patient support apparatus 20, and/or another object or structure within the healthcare facility.

In some embodiments, patient support apparatus 20 includes four UWB transceiver 132, each of which are positioned generally adjacent one of the four corners of patient support apparatus 20. In some such embodiments, the four UWB transceiver 132 are attached to, or positioned near, the four corners of litter frame 28. In other embodiments, the four UWB transceivers 132 are attached to, or positioned near, the four corners of base 22. In some embodiments, each of the four UWB transceivers 132 are attached to the corners of support deck 30. Still other locations of the UWB transceivers 132, as well as different numbers of the UWB transceiver 132, may be incorporated into patient support apparatus 20. In those embodiments of patient support apparatus 20 where one or more of the UWB transceivers 132 are coupled to components of patient support apparatus 20 that are movable (e.g. litter frame 28, which can have its height and orientation changed; or support deck 30 that can have its sections, such as head section 44, pivoted), sensors are included within patient support apparatus 20 that communicate the current position of the movable component to controller 140 so that controller 140 is able to determine the current positions of the UWB transceivers 132 and use those positions when determining the current location of an object, such as a badge 142, a tag 188, and/or a device 100.

Controller 140, as well as controller 112, may take on a variety of different forms. In the illustrated embodiment, each of these controllers is implemented as a conventional microcontroller. However, these controllers may be modified to use a variety of other types of circuits-either alone or in combination with one or more microcontrollers-such as, but not limited to, any one or more microprocessors, field programmable gate arrays, systems on a chip, volatile or nonvolatile memory, discrete circuitry, and/or other hardware, software, or firmware that is capable of carrying out the functions described herein, as would be known to one of ordinary skill in the art. Such components can be physically configured in any suitable manner, such as by mounting them to one or more circuit boards, or arranging them in other manners, whether combined into a single unit or distributed across multiple units. The instructions followed by controllers 112 and 140 when carrying out the functions described herein, as well as the data necessary for carrying out these functions, are stored in a corresponding memory that is accessible to that particular controller (e.g. memory 134 for controller 140, and a memory (not shown) for controller 112). In some embodiments, controller 140 may include and/or work with a microcontroller that is integrated into, or associated with, UWB transceiver(s) 132, and controller 112 may include and/or work with a microcontroller that is integrated into, or associated with, UWB transceiver 126.

Controller 140 utilizes UWB transceivers 132 to determine the relative position of patient support apparatus 20 with respect to one or more nearby locator units 60, one or more devices 100, one or more tags 188, and/or one or more badges 142. If patient support apparatus 20 is positioned within range of a locator unit 60, its UWB transceivers 132 communicate with the UWB transceiver 126 positioned on that locator unit 60, and the transceivers 132 and 126 exchange signals that enable them to determine the distances between themselves. This distance determination is done for each UWB transceiver 132 positioned onboard patient support apparatus 20 (or for as many as is necessary in order to determine an accurate position of locator unit 60 relative to patient support apparatus 20).

In some embodiments, UWB transceivers 126, 132 may also be configured to determine an angular relationships between themselves. The distance (and angle information) in at least some embodiments is calculated by UWB transceiver 132 and controller 140 of patient support apparatus 20. In other embodiments, UWB transceiver 126 and controller 112 may calculate the distance (and angle information) and forward the results of this calculation to patient support apparatus 20 (either via UWB transceiver 126 or BT transceiver 106). In either situation, patient support apparatus controller 140 is informed of the distances (and, in some embodiments, as noted, the angle information) between transceivers 132 and 126. These distances and orientations are then used to calculate a relative position of patient support apparatus 20 to the locator unit 60 in a common frame of reference that may be defined in a fixed relationship to the patient support apparatus 20 or in a fixed relationship to the locator unit 60.

Although FIGS. 4 and 5 only illustrate a single locator unit 60, it will be understood that a typical healthcare facility will include multiple locator units 60 positioned at different locations throughout the facility, including ones positioned within patient rooms and others positioned outside of patient rooms. Typically, at least one linked locator unit 60 will be positioned in each patient room of the healthcare facility, and if the patient room is intended to be occupied by more than one patient (e.g. it includes multiple bays), then additional linked locator units 60 may be included so that each patient support apparatus 20 will have a linked locator unit 60 positioned adjacent to each bay area in the room. Additional locator units 60, such as unlinked locator units 60a, may also be positioned at other locations through the healthcare facility.

The location of patient support apparatus 20 relative to locator units 60 is repetitively determined by an exchange of signals between UWB transceivers 126 and 132. This exchange is initiated by an interrogation signal that may be sent by the UWB transceivers 126 of the locator unit 60, and/or it may be sent by the UWB transceivers 132 of the patient support apparatuses 20. The trigger for sending these interrogation signals (from either source) may simply be the passage of a predefined interval of time, in at least some embodiments. That is, in some embodiments, patient support apparatus 20 and/or locator units 60 may be configured to periodically send out an interrogation signal that will be responded to by any UWB transceivers 126 or 132 that are positioned with range of that signal. In those embodiments where patient support apparatuses 20 are configured to send out such an interrogation signal, the time intervals between the interrogation signals may be varied depending upon the location, the number of devices 100 and/or badges 142 (if any) that are positioned within range of patient support apparatus 20, and/or the status of the patient support apparatus 20. For example, in some embodiments, controller 140 may be configured to send out the interrogation signals with longer timer intervals between them when the patient support apparatus is stationary (and, in some cases, when no devices 100 or badges 142 are currently positioned in communication range), and to send out the interrogation signals with shorter time intervals between them when the patient support apparatus 20 is in motion and/or when at least one object (device 100, badge 142, or tag 188) is currently positioned within communication range of transceivers 132. In any of the aforementioned embodiments, motion of the patient support apparatus 20 may be detected in any suitable manner, such as by including one or more motion sensors on the patient support apparatus 20 (e.g. one or more accelerometers), and/or by monitoring the values of the repetitive distance measurements and looking for changes indicative of movement.

The measured distances (and/or angular information between locator units 60 and patient support apparatuses 20) that are generated from the communications between UWB transceivers 126, 132 may utilize Angle of Arrival (AoA) information, Time of Flight (TOF) information, Channel State Information, Time Difference of Arrival (TDoA) information, Two-Way Ranging (TWR) ranging information, and/or other information. In some embodiments, each transceiver 126, 132 includes an array of antennas that are used to generate distance and/or angular information with respect to the transceivers 126, 132 in which it is in communication. Still further, in some embodiments, UWB transceivers 126, 132 include one or more of their own microcontrollers, and the location of UWB transceivers 126, 132 may be determined by these internal microcontrollers without utilizing controller 140 and/or 112. In other embodiments, controllers 112 and/or 140 may work in conjunction with the microcontrollers of transceivers 126, 132 to determine their relative locations to each other.

Nurse call interface 154 of patient support apparatus 20 (FIG. 5) includes Bluetooth transceiver 128 and a cable port 148, in some embodiments. Nurse call interface 154 provides an interface for patient support apparatus 20 to communicate with outlet 64 of nurse call system 70. That is, nurse call interface 154 provides the means for patient support apparatus 20 to bidirectionally communicate with communication outlet 64. As has been discussed, in some situations, patient support apparatus 20 uses Bluetooth transceiver 128 to communicate with Bluetooth transceiver 106 of linked locator unit 60, and linked locator unit 60 forwards communications back and forth between outlet 64 and patient support apparatus 20. In other words, in some situations, linked locator unit 60 functions as a communications intermediary between nurse call interface 154 and outlet 64. Alternatively, a nurse call cable 66 may be coupled directly between patient support apparatus 20 and wall outlet 64, thereby avoiding the need to use linked locator unit 60 as a communication intermediary. In such situations, one end of a nurse call cable 66 is plugged into cable port 148 of patient support apparatus 20 and the other end of the cable 66 is plugged directly into outlet 64. Nurse call interface 154 thereby provides patient support apparatus 20 with the ability to communicate either wirelessly or via wired means with the outlet 64.

Patient support apparatus 20 also includes, in at least some embodiments, a microphone 146 (FIG. 5) that is used to detect the voice of the patient when the patient wants to speak to a remotely positioned nurse. The patient's voice is converted to audio signals by microphone 146 and controller 140 is adapted to forward these audio signals to an adjacent communications outlet 64 positioned in wall 62 (FIG. 4). When a cable 66 is coupled between cable port 148 of patient support apparatus 20 and outlet 64, controller 140 forwards these audio signals to outlet 64 via the cable 66. When no such cable 66 extends between patient support apparatus 20 and outlet 64, controller 140 wirelessly forwards these audio signals to the linked locator unit 60 that it is currently associated with (using transceiver 128, or in some embodiments, one of transceivers 132) and controller 112 of linked locator unit 60 forwards these audio signals to outlet 64. As was noted, outlet 64 is in electrical communication with a conventional nurse call system 70 that is adapted to route the audio signals to the correct nurse's station 78, and/or other location. In some embodiments, microphone 146 acts as both a microphone and a speaker. In other embodiments, a separate speaker may be included in order to communicate the voice signals received from the remotely positioned nurse. In some embodiments, the audio communication between patient support apparatus 20 and communications outlet 64 is carried out in any of the manners, and/or includes any of the structures, disclosed in commonly assigned U.S. patent application Ser. No. 16/847,753 filed Apr. 14, 2020, by inventors Alexander Bodurka et al. and entitled PATIENT SUPPORT APPARATUSES WITH NURSE CALL AUDIO MANAGEMENT, the complete disclosure of which is incorporated herein by reference.

After the installation of locator units 60 in a particular healthcare facility, the location of each locator unit 60 within that facility is recorded. In some embodiments, the coordinates of the locations of locator units 60 are recorded in a common frame of reference (or converted to a common frame of reference after recordation). Such coordinates may be three dimensional (i.e. include a vertical and two horizontal components), or they may be two dimensional (no height component). In other embodiments, a more generalized location of one or more locator units 60 is determined, rather than the precise coordinates of the locator units 60. The generalized location of the locator units 60 may include an indication of the room, bay, area, hallway, portion of a hallway, wing, maintenance area, etc. that the locator unit 60 is positioned in. In still other embodiments, the locations of one or more locator units 60 are determined both generally and more precisely.

Regardless of how the location of each locator unit 60 is initially determined after they are installed in a healthcare facility (e.g. whether their coordinates are determined or a more generalized location is determined), the locations of all of the locator units 60, as well as their unique IDs 122, are stored in a memory accessible to server 84. Server 84 then uses this location data and ID data to determine the location of a patient support apparatus 20 (as well as the location of associated devices 100 and badges 142). Alternatively, or additionally, the location data and IDs 122 are forwarded to patient support apparatuses 20 for storage in their onboard memories 134 and for use in determining their own locations. In some embodiments, the location of each locator unit 60 (whether specific and/or general) may also, or alternatively, be stored in a memory within that particular locator unit 60 and shared with the devices it communicates with (e.g. patient support apparatuses 20). In some other embodiments, the location of each locator unit 60 may be stored in multiple locations.

It will be appreciated that patient support apparatuses 20 are configured to communicate with locator units 60 regardless of the orientation of the patient support apparatus 20. That is, the UWB transceivers 126 and 132 are radio frequency transceivers that do not rely on line of sight communication, unlike the IR transceiver 124 (if present). Thus, the patient support apparatuses 20 do not have to be pointed in any particular direction with respect to the locator units in order for transceivers 126 and 132 to communicate. This differs from some prior art systems that use IR communication between the patient support apparatuses 20 and the locator units and that require the IR transceiver onboard the patient support apparatus to be aimed toward the locator unit in order for communication to be established. It will also be understood that locator units 60 can be positioned on walls, columns, ceilings, or any other fixed structures within the healthcare facility.

Patient support apparatus 20 is also configured to use UWB transceivers 132 to determine the position of other object relative to patient support apparatus 20, such as one or more devices 100, one or more badges 142 and/or one or more tags 188 (FIG. 5). As will be discussed in greater detail below, controller 140 uses UWB transceivers 132 to repetitively determine the relative position of these object by communicating with one or more respective UWB transceivers that are part of these objects. Such UWB transceivers operate in the same manner as UWB transceivers 132 and/or UWB transceiver 126 of locator units 60. And, as will also be discussed in greater detail below, controller 140 of patient support apparatus 20 uses the relative position information to determine how it will interact with these objects, including whether to associate with these objects or not. When controller 140 associates patient support apparatus 20 with one or more of these objects, as will be discussed in greater detail below, controller 140 and/or server 84 may take one or more of the following actions: display data from these objects on display 52 and/or display device 56; send data from one or more of these objects to patient support apparatus server 84, EMR server 92, and/or badge sever 94 via network transceiver 96; retrieve data about one or more of these objects from patient support apparatus server 84, EMR server 92, and/or badge sever 94 via network transceiver 96; send one or more signals from these objects to communication outlet 64 (via a cable 66 or through linked locator unit 60); forward one or more signals from outlet 64 to one or more of these objects; retrieve data from EMR server 92 that was generated by these objects; retrieve data from these objects via another route that is independent from EMR server 92; and/or take other actions.

In the particular embodiment shown in FIG. 5, badge 142 includes a controller 156, a UWB transceiver 158, a microphone 160, a speaker 162, a network transceiver 164, and a unique ID 166. UWB transceiver 158 is adapted to communicate with the UWB transceivers 132 positioned onboard patient support apparatus 20 so that the position of badge 142 relative to patient support apparatus 20 can be repetitively determined. UWB transceiver 158 may be the same as all of the other UWB transceivers discussed herein (e.g. UWB transceivers 126, 132, etc.). UWB transceiver 158 is further adapted to transmit the unique ID 166 of badge 142 to patient support apparatus 20 so that patient support apparatus 20 knows which specific badge 142 it is communicating with. It will be understood that the components of badge 142 shown in FIG. 5 are merely an illustrative example of a badge 142, and that different badges may be utilized with the communication system of the present disclosure that have fewer, greater, or different components than those shown in FIG. 5. Those badges 142 that include a UWB transceiver 158 and identifier 166 are able to fully operate in accordance with the principles discussed herein. However, badges that do not include a UWB transceiver 158 can still be integrated into the communication system of the patient support apparatuses 20 and/or patient support apparatus server 84 described herein.

Controller 156 of badge 142 is adapted to oversee the operation of badge 142, process the communications of UWB transceiver 158 with other UWB transceivers (e.g. transceivers 132), respond to the activation of controls on badge 142 (if any), and oversee the operation of microphone 160 and speaker 162. Badge 142 is designed to be worn by a healthcare facility worker, or attached to a structure (e.g. necklace, bracelet, clip, etc.) that is adapted to be worn by a healthcare facility worker. Badge 142 of FIG. 5 is adapted to allow a caregiver to speak into microphone 160 and have his or her voice transmitted to another badge 142, and/or a computing device in communication with badge server 94. Similarly, badge 142 of FIG. 5 is adapted to receive audio signals from other badges 142, or computing devices, and to route them to speaker 162 so that the person wearing badge 142 can hear those audio signals. In other words, badge 142 is adapted to allow its wearer to communicate with remotely positioned personnel who are either wearing another badge 142 or who are able to access badge server 94 and communicate audio signals to/from server 94 using a conventional computing device (e.g. a desktop computer with a headset having a microphone and speakers).

Badge 142 communicates audio signals by routing them through badge server 94 using network transceiver 164. Network transceiver 164, like network transceiver 96 of patient support apparatus 20, may be a WiFi transceiver (e.g. IEEE 802.11) that wirelessly communicates with one or more conventional wireless access points 82 of local area network 80. In other embodiments, network transceiver 164 may be a wireless transceiver that uses conventional 5G technology to communicate with network 80 and badge server 94. In still other embodiments, network transceiver 164 may be a cellular transceiver that is adapted to use one or more protocols of conventional cell phones to communicate with badge server 94.

Regardless of the specific technology of network transceiver 164, when a user of a badge 142 calls another caregiver, controller 156 sends the user's audio signals to badge server 94 and badge server 94 routes those audio signals to the appropriate recipient (which, as noted, may be another badge 142 or a computing device in communication with badge server 94). Similarly, the recipient's audio signals are routed to badge server 94 and badge server 94 then forwards those to badge 142. In this manner, two-way voice communication is established between a first badge 142 and another badge 142 or computing device.

Controller 140 of patient support apparatus 20 repetitively determines the position of badges 142 after they move within communication range of UWB transceivers 132. Controller 140 uses UWB transceivers 132, which communicate with the UWB transceiver 158 of each badge 142, to repetitively determine the relative position of the badge 142 with respect to patient support apparatus 20. Controller 140 compares this relative position to a space volume 152c (FIGS. 4 & 6) and, if the badge 142 is outside of space volume 152c, it automatically disassociates badge 142 from patient support apparatus 20. On the other hand, as long as the position of badge 142 remains inside of space volume 152c, controller 140 maintains the association of badge 142 with patient support apparatus 20. As will be discussed in greater detail below, once a badge 142 is associated by controller 140 with a patient support apparatus 20, controller 140 may take one or more automatic actions, several of which will now be discussed.

In some embodiments, after controller 140 associates a badge 142 with patient support apparatus 20, it is configured to automatically determine a class of user to which the badge 142 belongs, and/or to send the badge ID 166 to patient support apparatus server 84 for patient support apparatus server 84 to determine the corresponding user class. That is, as discussed more below, the unique ID 166 of each badge 142 may contain information that indicates which class of healthcare worker the badge 142 belongs to. Alternatively, it may be necessary to determine the class of worker to which a badge 142 belongs by utilizing the identity of the caregiver who is currently using that badge 142. In such cases, patient support apparatus server 84 is configured to receive information from one or more other servers on network 80 that store data indicating which workers within that particular healthcare facility belong to which classes of (e.g. a worker-ID-to-worker-class database). Such servers may include, but are not limited to, caregiver assignment server 104 and/or another server. In addition to determining the class of a particular healthcare worker, patient support apparatus server 84 is configured to utilize data that correlates badge IDs 166 to specific healthcare workers (e.g. a worker-ID-to-badge-ID database). Such data will typically be stored in badge server 94. By combining the data that correlates healthcare worker identities with the badge identifiers 166, along with data from badge server 94 that correlates badge identifiers 166 with healthcare worker identities, patient support apparatus server 84 is able to determine which class of healthcare worker a given badge 142 corresponds to.

The different classes of workers who wear badges 142 may vary from healthcare facility to healthcare facility. In one embodiment, the different classes of users may correspond to nurses, nursing assistants, doctors, service technicians, cleaning workers, and transportation worker (the latter of which are responsible for transporting patients to different locations within the healthcare facility). Server 84 may also be customizable to allow authorized personnel of a healthcare facility to define their own classes, and in some instances, such personnel may further refine one or more of the aforementioned classes. For example, some healthcare facilities may assign classes to certain types of doctors and/or nurses, such as emergency room doctors and/or nurses, pediatric doctors and/or nurses, cardiac doctors and/or nurses, etc. Other classification systems can, of course be used. Also, as noted above, the classification of healthcare workers may be defined on another server within the healthcare facility and read by patient support apparatus server 84. Alternatively, the classes of healthcare workers may be entered directly into patient support apparatus server 84 by authorized personnel, such as by utilizing a conventional computing device (e.g. computer 168 (FIG. 8)) that has access to network 80 and patient support apparatus server 84.

Regardless of the specific classifications that are implemented in a healthcare facility, patient support apparatus server 84 and/or controller 140 are configured to determine which class each badge 142 belongs to using the badge ID 166 and the other information mentioned above (a worker-ID-to-badge-ID database and a worker-ID-to-worker-class database). After making this determination, in some embodiments, controller 140 and/or patient support apparatus server 84 are configured to make one or more changes to patient support apparatus 20 in response to that determination, and/or to automatically take one or more other actions.

For example, in some embodiments, controller 140 and/or patient support apparatus server 84 are configured to automatically use the object IDs and their position information to determine if patient support apparatus 20 has been cleaned, is ready to be cleaned, and/or if the patient is ready to be transported, or has been transported. Controller 140 and/or patient support apparatus server 84 may also, or alternatively, be configured to use the object IDs to determine where objects added to the patient support apparatus 20 are positioned, as well as which healthcare worker added those objects. These automatic actions are described in greater detail further below.

Controller 140 of patient support apparatus 20 is also configured to automatically associate and disassociate patient support apparatus 20 from other objects besides badges 142, such as one or more devices 100 and/or tags 188. An example of such a device 100 is depicted generically in FIGS. 4 and 5. Device 100 includes, at a minimum, a UWB transceiver 178 and a unique ID 180 (FIG. 5). Device 100 may also, depending upon the particular device 100, include a device controller 182 and/or one or more sensors 184. Device 100 may also include additional structures beyond those shown in FIG. 5.

The types of devices 100 that may be incorporated into the communication system described herein include, but are not limited to, any one or more of the following: another patient support apparatus 20, an infusion pump, a vital sign sensor, an exercise device, a heel care boot, an IV stand and/or pole, a ventilator, a DVT pumps, a patient monitor (e.g. a saturated oxygen (SpO₂) monitor, an EKG monitor, a vital sign monitor, etc.), a patient positioning devices (e.g. a wedge, turning device, pump), an ambient sensor (e.g. air temperature, air flow, light, humidity, pressure, altitude, sound/noise), a mattress 42, a portable exit detection sensor, an attachable nurse call device, an incontinence pad or one or more sensors adapted to detect patient incontinence, a Holter device adapted to monitor and record a patient's heart signals, a patient ID tag or bracelet worn by the patient that identifies the patient, a caregiver tag or ID bracelet worn by a caregiver that identifies the caregiver, a patient temperature management device (or associated device, such as a one or more hoses, thermal wraps, etc.), one or more mobility assistance devices that a patient may be expected to use, and/or still other types of devices 100.

In those embodiments where devices 100 include an infusion pump, patient support apparatus 20 and/or patient support apparatus server 84 may be configured to carry out any of the functions associated with the infusion pump that are described in commonly assigned U.S. patent application Ser. No. 63/349,369 filed Jun. 6, 2022, by inventors Krishna Bhimavarapu et al. and entitled COMMUNICATION SYSTEM FOR PATIENT SUPPORT APPARATUSES, the complete disclosure of which is incorporated herein by reference.

In those embodiments where devices 100 include a portable exit detection sensor, another patient support apparatus, and/or an attachable nurse call device, patient support apparatus 20 and/or patient support apparatus server 84 may be configured to carry out any of the functions associated with the portable exit detection sensors, nurse call devices, and secondary patient support apparatuses disclosed in commonly assigned U.S. patent application Ser. No. 63/352,061 filed Jun. 14, 2022, by inventors Jerald Trepanier et al. and entitled COMMUNICATION SYSTEM FOR PATIENT SUPPORT APPARATUSES, the complete disclosure of which is incorporated herein by reference.

In those embodiments where devices 100 include one or more vital sign sensors, patient support apparatus 20 and patient support apparatus server 84 may be configured to carry out any of the functions associated with the vital sign sensors and/or display devices disclosed in commonly assigned U.S. patent application Ser. No. 63/306,279 filed Feb. 3, 2022, by inventors Madhu Thota et al. and entitled COMMUNICATION SYSTEM FOR PATIENT SUPPORT APPARATUSES, the complete disclosure of which is incorporated herein by reference.

In those embodiments where devices 100 include one or more patient temperature management devices, patient support apparatus 20 and/or patient support apparatus server 84 may be configured to carry out any of the functions associated with the patient temperature management devices disclosed in commonly assigned U.S. patent application Ser. No. 63/314,221 filed Feb. 25, 2022, by inventors Jerry Trepanier et al. and entitled COMMUNICATION SYSTEM FOR PATIENT SUPPORT APPARATUSES AND TEMPERATURE MANAGEMENT DEVICES, the complete disclosure of which is incorporated herein by reference.

In those embodiments where one or more of the tags 188 are worn by patients, patient support apparatus 20 and/or patient support apparatus server 84 may be configured to carry out any of the functions associated with patient monitoring that are described in commonly assigned U.S. patent application Ser. No. 63/245,279, filed Sep. 17, 2021, by inventors Jerry Trepanier et al. and entitled PATIENT SUPPORT APPARATUSES WITH PATIENT MONITORING, the complete disclosure of which is incorporated herein by reference.

As shown in FIG. 5, tag 188 includes a controller 210, an ultra-wideband (UWB) transceiver 212, and a unique ID 214. Controller 210 is adapted to oversee the operation of UWB transceiver 212 and may take on the same form as any of the controllers previously described herein. UWB transceiver 212 is adapted to communicate with UWB transceivers 132 onboard patient support apparatus 20 in the same manner as the UWB transceivers 158 of badges 142. That is, controller 140 of patient support apparatus 20 is configured to automatically communicate with, and repetitively determine the relative position of, UWB transceivers 212 when tags 188 are positioned within communication range of UWB transceivers 132. Controller 210 of tag 188 is configured to share unique ID 214 with UWB transceivers 132 of patient support apparatus 20 so that controller 140 is able to uniquely identify each tag 188 that is within communication range.

Tag 188 is adapted to be physically coupled to an object 190. To that end, tag 188 may include adhesive and/or mechanical fasteners, and/or other types of fastening mechanisms, for securing it to object 190. Object 190 may be any item whose position relative to patient support apparatus 20 and/or within the healthcare facility is desirably known. In some embodiments, tags 188 may take on any of the forms of the tags described in commonly assigned U.S. patent application Ser. No. 63/193,777 filed May 27, 2021, by inventors Thomas Deeds et al. and entitled SYSTEM FOR ASSOCIATING MEDICAL DEVICE DATA, the complete disclosure of which is incorporated herein by reference.

The identifiers 214 of tags 188, as well as the identifiers 180 of devices 100 and identifiers 166 of badges 142, may be structured such that controller 140 of patient support apparatus 20 can automatically use these identifiers to determine the type of object the identifier is associated with. That is, for example, if all IDs are, say, sixteen characters long, the first four characters may provide an indication of the type of object. Patient worn tags may therefore have a unique first four characters in their ID 214 that identify them as patient tags 188; ventilators 100 may have another unique first four characters in their ID 180 that identify them as ventilators; infusion pumps 100 may have yet another unique first four characters in their ID 180 that identify them as infusion pumps; and so on. The unique IDs 166 of badges 142 may utilize a similar concept, but instead of identifying a type of object, they may be defined in such a way that their unique IDs 166 identify a type of healthcare worker (as well as uniquely identifying that worker). The different types of healthcare workers that may be present in a particular healthcare facility may vary, and several examples are discussed elsewhere herein. It will of course be understood that the aforementioned examples of IDs 166, 180, and 214 are merely illustrative examples, and that other ways of organizing and/or defining these identifiers may be implemented.

Tags 188 (FIG. 5) are adapted to be physically coupled to an object 190. To that end, tag 188 may include adhesive and/or mechanical fasteners, and/or other types of fastening mechanisms, for securing it to object 190. Object 190 may be any item whose position relative to patient support apparatus 20 and/or within the healthcare facility is desirably known. In some embodiments, tags 188 may take on any of the forms of the tags described in commonly assigned U.S. patent application Ser. No. 63/193,777 filed May 27, 2021, by inventors Thomas Deeds et al. and entitled SYSTEM FOR ASSOCIATING MEDICAL DEVICE DATA, the complete disclosure of which is incorporated herein by reference.

Controller 140 of patient support apparatus 20 automatically associates patient support apparatus 20 with, and disassociates patient support apparatus 20 from, one or more objects (devices 100, badges 142, and/or tags 188) by repetitively determining the relative location of the UWB transceivers associated with these objects (e.g. UWB transceiver 178 of device 100, UWB transceiver 158 of badge 142, and UWB transceiver 212 of tag 188) with respect to patient support apparatus 20. As will be discussed in greater detail below, controller 140 may associate the object to patient support apparatus 20 if it moves inside of one of the space volumes 152 (also discussed in more detail below), and disassociate the object if it moves outside of one of these space volumes 152.

Either in addition to, or in lieu of, of the automatic association of some objects (devices 100, badges 142, and/or tags 188) with patient support apparatus 20, controller 140 may be configured to allow one or more specific objects to be associated with patient support apparatus 20 through a manual process. That is, some objects may be associated with patient support apparatus 20 in a manual process, some objects may be associated in an automatic process, and some objects may be associated in both a manual and an automatic process. For the manual process, the caregiver manually informs patient support apparatus 20 that a particular object should be associated with that patient support apparatus 20. This manual process may be accomplished in different manners. For example, in some embodiments, the object and patient support apparatus 20 may include near field transceivers that, when positioned within close proximity (e.g. several inches) of each other, exchange information that establishes that that particular object should be associated with that particular patient support apparatus 20. Further details regarding the use of near field transceivers for associating objects with patient support apparatus 20 are disclosed in commonly assigned U.S. patent application Ser. No. 63/352,061 filed Jun. 14, 2022, by inventors Jerald Trepanier et al. and entitled COMMUNICATION SYSTEM FOR PATIENT SUPPORT APPARATUSES, the complete disclosure of which has already been incorporated herein by reference. Other types of manual associations processes may also, or alternatively be used, some of which are also disclosed in the aforementioned '061 application.

For the automatic association process, as noted previously, controller 140 is configured to automatically associate at least some objects with patient support apparatus 20 based upon the proximity of those objects to patient support apparatus 20. This automatic association may be accomplished by the object moving inside of a predetermined volume of space 152 defined with respect to patient support apparatus 20 (e.g. one of space volumes 152a-c, or another space volume; FIG. 6). Once inside the space, controller 140 automatically associates the object with patient support apparatus 20.

Controller 140 is also configured to automatically disassociate one or more objects (devices 100, badges 142, and/or tags 188) from patient support apparatus 20. The automatic disassociation may be accomplished by the object moving outside of the same predetermined volume of space 152. Alternatively, controller 140 may use modified volumes of space-such as, but not limited to, larger space volumes-when automatically determining whether to disassociate one of these objects from patient support apparatus 20. In other words, once an object has been determined to be positioned inside of a particular volume of space, such as space volumes 152a-c (and any additional association conditions are met, if there are any), and controller 140 has associated the object with patient support apparatus 20, controller 140 may thereafter increase the size of—and/or otherwise change one or more dimensions of—the volume of space when determining whether to disassociate the object. In this manner, the volumes of space 152 may have a sort of hysteresis aspect wherein an object has to be positioned inside of a smaller space volume in order to be associated with patient support apparatus 20, but thereafter can only be disassociated if it moves outside of a larger sized volume of space. In still other embodiments, the dimensions of one or more of the volumes of space are the same for both association and disassociation purposes.

Once an object is associated with patient support apparatus 20, it thereafter remains associated with patient support apparatus 20 until it moves outside of a volume of space, such as space 152b of FIGS. 4 & 6, at which point controller 140 disassociates the object from patient support apparatus 20. Controller 140 repetitively determines and monitors the position of the object while it is within communication range of controller 140. Specifically, controller 140 uses UWB transceivers 132, which communicate with the object's UWB transceiver (UWB transceiver 178 for device 100, UWB transceiver 158 for badge 142, and UWB transceiver 212 for tag 188) to repetitively determine the relative position of the object with respect to patient support apparatus 20. Controller 140 compares this relative position to a particular space volume 152 and, if it is outside of the space volume 152, it automatically disassociates the object from patient support apparatus 20. On the other hand, as long as the position of the object remains inside of the space volume 152, controller 140 maintains the association of the object with patient support apparatus 20. As noted previously, the dimensions of the space volume 152 that are used for the association/dissociation process of the object may be the same as, or different from, the dimensions of space volume 152 that are used for the association/dissociation process.

Display device 56 (FIG. 5) includes a display controller 192 and a display 194. Display device 56 differs from the other objects discussed herein in that it does not need to include a UWB transceiver, such as is found in these other objects. This is because patient support apparatus 20 is not configured to determine the location of display device 56. Instead, controller 140 of patient support apparatus 20 is configured to use UWB transceivers 132 to determine the location of the locator unit 60 to which the display device 56 is coupled. If the patient support apparatus 20 (or a reference point on it) and locator unit 60 are not both positioned within the space volume 152a, the controller 140 will not send data to be displayed on display device 56 to the locator unit 60. If the locator unit 60 and patient support apparatus 20 (or a reference point on it) are both positioned within the space volume 152a, controller 140 is configured to be able to send data to be displayed on the coupled display device 56 by forwarding the data to be displayed to the associated locator unit 60, which then forwards it to display device 56 via video port 120 and video cable 110. In some embodiments, controller 140 forwards the data to be displayed on display device 56 to the associated locator unit 60 by using Bluetooth transceiver 128, while in other embodiments it may use a UWB transceiver 132, or another type of transceiver. The forwarding of data to be displayed on display device 56 is a direct forwarding of data from patient support apparatus 20 to locator unit 60, and from there to display device 56, with no other intermediaries, in at least some embodiments.

Because display device 56 need not include a UWB transceiver, display device 56 may be a conventional television, computer monitor, or other conventional device, that is capable of displaying the video signals that are transmitted over video cable 110. In addition, it is not necessary for display device 56 to execute any specialized software app that authenticates communication with the coupled locator unit 60 and/or with patient support apparatus 20. Instead, once controller 140 determines that locator unit 60 is within space volume 152a (or vice versa), controller 140 need not perform any additional security protocols regarding display device 56. Indeed, controller 140 does not need to be able to perform any communications with display device 56 other that forwarding data to be displayed to the associated locator unit 60. In this manner, display device 56 need not know anything about locator unit 60 and/or patient support apparatus 20. It merely displays the video signal coming from video cable 110, and therefore does not need a software app that is specialized to patient support apparatus 20 and/or to locator unit 60 and/or to UWB communications.

It should also be noted that the display of data on display 52 and/or display device 56 from an associated object is carried out by patient support apparatus 20 automatically. That is, when a user has configured patient support apparatus 20 to display data from a device 100 on a display device 56, for example, controller 140 automatically forwards this data to the associated display device 56. For example, if device 100 is a heart rate monitor and a patient coupled to device 100 is wheeled into a bay area of a patient room that includes a display device 56 coupled to a locator unit 60, controller 140 is configured to automatically start displaying the patient's heart rate information on that display device 56 as soon as patient support apparatus 20 completes the following two association processes: (1) the association between patient support apparatus 20 and the locator unit 60 to which display device 56 is coupled, and (2) the association between patient support apparatus 20 and the heart rate monitor. In this manner, the caregiver sees the patient's heart rate data displayed on the display device 56 within seconds after moving the patient support apparatus 20 into the bay area, and the caregiver doesn't need to connect any cables, press any buttons, or take any other actions, in order for the heart rate data to be displayed on display device 56. Similarly, when the patient support apparatus 20 moves out of the bay area, the display of the patient's data on the display device 56 automatically terminates. This automatic termination is carried out after patient support apparatus 20 moves outside of the space volume 152a associated with the locator unit 60 to which the display device 56 is connected. Once outside of this space volume 152a, controller 140 disassociates patient support apparatus 20 from that locator unit 60 and its associated display device 56, and once this disassociation occurs, it stops sending data to be displayed on display device 56.

As another example of the automatic display of device data on a display, if a caregiver approaches a patient support apparatus 20 while carrying a portable electronic device 98—such as a smart phone or tablet computer that includes a display, a UWB transceiver, and the appropriate software app—controller 140 is adapted to automatically start forwarding the data to the electronic device 98 for display on its screen. In this manner, a caregiver that brings his or her portable electronic device 98 within space volume 152b (or another space volume defined for such devices 98) can have that patient's heart rate data (and/or other data, such as from another object) automatically displayed on his/her device 98. When the caregiver moves his or her portable electronic device 98 outside of the space volume 152b, the display of that patient's data automatically ceases because controller 140 automatically disassociates patient support apparatus 20 from the portable electronic device 98 and stops sending it data to be displayed. If the caregiver then moves to another patient's patient support apparatus 20 and steps inside the space volume 152b of that patient support apparatus, he or she will automatically see the data from the objects associated with that patient displayed on his or her portable electronic device 98.

In some embodiments, controller 140 may be configured to display data from an object associated with patient support apparatus 20, and/or process the data from the associated object, in any of the manners disclosed in commonly assigned U.S. patent application Ser. No. 63/306,279 filed Feb. 3, 2022, by inventors Madhu Sandeep Thota et al. and entitled COMMUNICATION SYSTEM FOR PATIENT SUPPORT APPARATUSES, the complete disclosure of which is incorporated herein by reference.

In some embodiments, patient support apparatus 20 is configurable by a user (e.g. via control panel 54a) to control what information, if any, patient support apparatus 20 will send to an associated display device 56 (FIG. 5), to patient support apparatus server 84, to EMR server 92, and/or to another server on network 80. Thus, a user can instruct patient support apparatus 20, for example, to send blood pressure readings, breathing rate readings, and pulse rate readings from devices 100 that are vital sign sensors (and that are associated with patient support apparatus 20) to EMR server 92, but not status data from an object that doesn't generate data for recordation in EMR server 92. Patient support apparatus 20 may also be configured by the user to route data for displays 52 and/or 56 and/or one or more servers on network 80 in still other manners.

Locator units 60 are configured to send a message to patient support apparatuses 20 indicating whether or not a display device 56 (FIGS. 4-5) is coupled thereto via video port 120. In such embodiments, when controller 140 of patient support apparatus 20 receives the signal indicating that a display device 56 is coupled to an associated locator unit 60, it is configured to consult the customized user settings to determine what data, if any, to send to that locator unit 60 for forwarding to the display device 56 coupled to video port 120. It bears noting that, when patient support apparatus 20 associates itself with a particular locator unit 60 that has a display device 56 coupled to its video port 120, it is not necessary for the display device 56 to be positioned within space volume 152a in order for controller 140 to send data to be displayed on the display device 56. Instead, controller 140 is configured to only check whether the locator unit 60 is associated with patient support apparatus 20 or not. If it is, controller 140 may send (depending upon the user settings) data to the locator unit 60 for forwarding to the display device 56. If locator unit 60 is not associated with patient support apparatus 20, then controller 140 does not send any data to the locator unit 60 for displaying on display device 56.

In addition to allowing a user to customize what data, if any, that is sent to a display device 56, controller 140 is further configured to allow a user to customize what data is sent to different types of display devices 56. Thus, controller 140 may be programmed to allow a user to send a first set of data to a display device 56 that is coupled to an associated locator unit 60 and a second set of data, different from the first set of data, to a different display, such as, for example, display 52 of patient support apparatus 20 and/or another display, such as, but not limited to, ones that are incorporated into electronic device 98 (e.g. conventional smart phones, laptop computers, tablet computers, smart TVs and/or smart monitors) that are either in communication with patient support apparatus server 84 and/or that are positioned within space volume 152, as determined by one or more UWB transceivers coupled to them that are adapted to communicate with UWB transceiver 132 of patient support apparatus 20. It will be appreciated, that in such cases, the conventional smart phone, laptop computer, tablet computer, or other type of display device will include a software app that oversees the UWB communications with patient support apparatus 20 and that controls the display of the data on the display (i.e. screen) of the display device. The software app includes one or more security features built into it that only allow authorized users to have this data displayed on the display device, thereby preventing unauthorized users from viewing this data on their own smart phone, laptop computer, tablet computer, etc. In some embodiments, controller 140 is configured to execute a security check protocol with the display device to authenticate the display device prior to sending it any data for displaying thereon.

Patient support apparatus 20 may include one or more screens that are displayable on display 52 that allow a user to customize the data from associated objects that is shown on display 52 and/or display devices 56. In addition, patient support apparatus 20 is configured to allow the user to change the format and/or layout in which the selected data is to be displayed.

FIGS. 4 and 6 illustrate several of the different volumes of space 152 that controller 140 may use when carrying out the functions described herein. As was noted, in some embodiments, space volumes 152a-c are defined with respect to patient support apparatus 20 and therefore move as patient support apparatus 20 moves. Space volume 152a, which may be the smallest of the space volumes 152, is generally used for the automatic association and disassociation between patient support apparatus 20 and a locator unit 60. Space volume 152b is generally used for the automatic association and disassociation between patient support apparatus 20 and a variety of different devices 100 or tags 188 that are typically used in close proximity to the patient. Space volume 152c is generally used by controller 140 for the automatic association and disassociation between patient support apparatus 20 and badges 142 and/or tags 188 that may travel further away from patient support apparatus 20 (e.g. patient-worn tags 188). It will be understood that controller 140 may utilize space volumes 152 other than the three shown in FIGS. 4 and 6.

Space volume 152c is generally sized such that it encompasses substantially all of the room 58 in which patient support apparatus 20 is positioned, or at least that portion of the room in which a healthcare worker with a badge 142 (or a patient with a tag 188) is expected to be present, particularly while the worker attends to the patient assigned to patient support apparatus 20 and/or to patient support apparatus 20 itself. In some situations, space volume 152c may be large enough to extend into adjacent rooms 58, but this will not affect the proper association of a badge 142 with patient support apparatus 20 so long as access to those adjacent rooms cannot be obtained without the healthcare worker or patient first exiting from space volume 152b before traveling to those adjacent rooms. In other words, space volume 152c may be advantageously defined such that, when the person's badge 142 (or patient's tag 188) is moved out of room 58 through a doorway, the badge 142 will necessarily move outside of space volume 152c, thereby causing it to become disassociated from the patient support apparatus 20 before the badge 142 or tag 188 enters the space volume 152c associated with another patient support apparatus 20.

In some embodiments, any of space volumes 152a-c may be defined with one or more static dimensions. In other embodiments, patient support apparatus 20 and/or patient support apparatus server 84 may be configured to allow authorized individuals to change one or more dimensions of space volumes 152a-c. Still further, in some embodiments, space volumes 152a-c may have variable dimensions based upon the specific room, bay, or other location, in which patient support apparatus is currently positioned. In these embodiments, controller 140 may utilize a table stored in memory 134 that defines the dimensions of any of space volumes 152a-c based on the current location of patient support apparatus 20. The location of patient support apparatus 20 may be determined by controller 140 from the locator unit ID 122 that it receives from an associated locator unit 60, and/or it may be derived from information received from patient support apparatus server 84.

FIG. 6 illustrates an example of an arbitrary healthcare facility room 58 showing a patient support apparatus 20, a device 100, a badge 142, a linked locator unit 60, and an unlinked locator unit 60a. FIG. 6 also illustrates first volume of space 152a, second volume of space 152b, and third volume of space 152c. First volume of space 152a, as has been discussed, is used to determine whether patient support apparatus 20 should associate or disassociate itself from the locator unit 60 that is positioned adjacent to, or inside of, first volume of space 152a. Second volume of space 152b is used by controller 140, as has been noted, to determine whether to associate or disassociate certain types of devices 100 and/or tags 188 (those that are expected to be used in close proximity to patient support apparatus 20) with patient support apparatus 20. Third volume of space 152c may be used by controller 140 to determine whether to associate or disassociate one or more badges 142 with patient support apparatus 20, and/or one or more devices 100 and/or tags 188 that are expected to be used a further distance away from patient support apparatus 20. Second volume of space 152b is generally smaller than third volume of space 152c. It can therefore be seen that controller 140 of patient support apparatus 20 may be configured to use multiple different volumes of space 152a-c for determining whether to associate an object with patient support apparatus 20.

As shown in FIG. 6, first volume of space 152a is dimensioned so that it will encompass fixed locator unit 60 when patient support apparatus 20 is positioned within about 5 to 10 feet of a locator unit 60. Second volume of space 152b encompasses patient support apparatus 20 and a relatively small amount of space surrounding patient support apparatus 20. This small amount of space around the perimeter of patient support apparatus 20 may be defined with the goal of encompassing IV poles, infusion pumps, or other objects that may be used in the care of the patient, but that may not necessarily be directly supported on patient support apparatus 20. Second volume of space 152b may also be defined such that it is unlikely to encompass objects that are being used on a patient assigned to another patient support apparatus 20 that may be positioned nearby. Third volume of space 152c encompasses a substantial portion of room 58. It will, of course, be understood that the sizes and dimensions of volumes of space 152a-c may vary from what is shown in the attached drawings.

As shown in FIG. 6, a fourth volume of space 152d may also be defined for locator units, such as unlinked locator units 60a, that are positioned in hallways, and/or in other areas outside of patient rooms 58. Space volumes 152d may be sized to provide more generalized locations of patient support apparatuses 20 (and the devices associated therewith), such as being located in a particular hallway, or portion of a hallway, a particular storage area, etc. In some embodiments, space volume 152d is used by controller 140 when determining whether to associated with an unlinked locator unit 60a. In some embodiments, the dimensions of space volumes 152d for each unlinked locator unit 60a are stored within a memory of each unlinked locator unit 60a and transmitted to patient support apparatus 20 when patient support apparatus 20 is within communication range. Alternatively, the dimensions of space volume 152d may be stored onboard patient support apparatus 20 and consulted by controller 140 based upon the ID 122 received from an unlinked locator unit 60a.

It will be understood that the dimensions of space volume 152d, like those of space volumes 152a-c, may be variable, user-customizable, location-dependent, object-dependent, and/or different from what is shown in FIG. 6. It will also be understood that, although the accompanying drawings all depict all of the space volumes 152a-d as having rectangular shapes, these shapes may be varied, including shapes that are all curved and/or shapes that have a combination of curved and straight boundaries. It will also be understood that the volumes of space 152a-c may be alternatively referred to as threshold distances from patient support apparatus 20. If the threshold distance has a constant value in all directions from a measurement point of the patient support apparatus 20, the volume of space 152 will define a sphere. If the threshold distance has a non-constant value in one or more different directions from the measurement point of the patient support apparatus 20, the volume of space 152 will define a non-spherical shape.

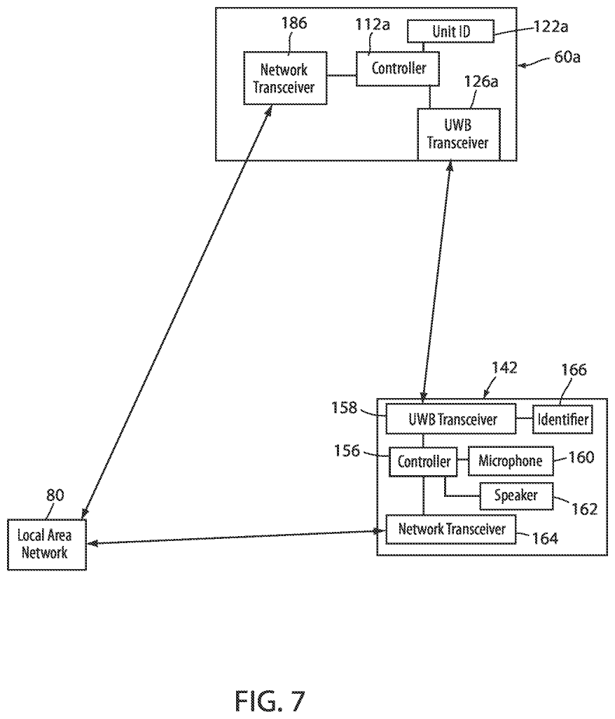

FIG. 7 illustrates an example of the internal components of an unlinked locator unit 60a (as well as a badge 142 and local area network 80). Unlinked locator unit 60a includes a number of components that are the same as those found in linked locator units 60. Those common components are labeled with the same reference number followed by the letter “a.” Components of unlinked locator units 60a that are not found in linked locator units 60 are provided with a new reference number.

Unlinked locator unit 60a includes a controller 112a, a UWB transceiver 126a, a unit ID 122a, and, in some embodiments, a network transceiver 186. Network transceiver 186 is, in at least one embodiment, a WiFi transceiver adapted to be able to communicate with network 80 via wireless access points 82. Controller 112a is adapted to determine the location of badges 142 that are positioned within range of UWB transceiver 126a. In some embodiments, unlinked locator unit 60a includes multiple UWB transceivers 126a, while in other embodiments, unlinked locator unit 60a may include only a single UWB transceiver 126a. Regardless of how many UWB transceivers 126a unlinked locator unit 60a includes, controller 112a is configured to use UWB transceiver(s) 126a to measure the distance between itself and any UWB objects (e.g. devices, 100, badges 142 and/or tags 188) that are positioned within range of unlinked locator unit 60a. In some situations, such as where unlinked locator unit 60a includes only a single UWB transceiver 126a, controller 112a may only be able to determine a distance between itself and the object, rather than a three-dimensional position. In other situations, such as where unlinked locator unit 60a includes multiple UWB transceivers 126a, controller 112a may be able to determine a three-dimensional position of a badge 142 relative to unlinked locator unit 60a.

Regardless of the 1-dimensional, 2-dimensional, or 3-dimensional position determination of an object, controller 112a is configured to send a message to patient support apparatus server 84 via network transceiver 186 that identifies the detected location of any badge 142 with respect to unlinked locator unit 60a. This message also identifies the unique identifier 122a of the unlinked locator unit 60a. Patient support apparatus server 84 is therefore able to determine the general location of the badge 142 within the healthcare facility using the stored positions of each locator unit 60 within the healthcare facility.

Alternatively, or additionally, to the determination of an object's location by controller 112a, the object (device 100, badge 142, and/or tag 188) may be configured to determine its location with respect to the unlinked locator unit 60a. When the object determines its location, it may forward it to patient support apparatus 20 via its UWB transceiver, and/or it may forward its location to unlinked locator unit 60a via its UWB transceiver, and/or, if the object includes a network transceiver, such as some badges 142, it may forward its location to patient support apparatus server 84 using its network transceiver. It can therefore be seen that the position of an object relative to an unlinked locator unit 60a may be determined by the unlinked locator unit 60a (i.e. its controller 112a) and/or it may be determined by the object itself.

In addition to tracking the position of objects as they move through the healthcare facility, unlinked locator units 60a may also be provided within a healthcare facility in order to provide location information to patient support apparatuses 20 as they are moved outside of the rooms within the healthcare facility. Patient support apparatus 20, in some embodiments, is adapted to communicate with UWB transceiver 126a of unlinked locator units 60a in the same manner that it communicates with UWB transceiver 126 of linked locator units 60. This enables patient support apparatus 20 to determine its relative position with respect to the unlinked locator unit 60a (which, in some embodiments, is a three-dimensional position). Controller 140 can therefore determine its position within the healthcare facility, and/or forward the locator ID 122a from the unlinked locator unit 60a to patient support apparatus server 84, and patient support apparatus server 84 can utilize this information to determine the current location of patient support apparatus 20 within the healthcare facility.

Patient support apparatus server 84 repetitively receives position information about all of the UWB objects (devices 100, badges 142, and/or tags 188) that are currently positioned in communication range of the UWB transceivers 132 onboard the multiple patient support apparatuses 20 that are positioned within the healthcare facility. Patient support apparatus server 84 may also receive position information from locator units 60 whose UWB transceivers 126a (FIG. 7) are positioned within communication range of one or more badges 142. Patient support apparatus server 84 is therefore repetitively provided with updates about the current location of all the UWB objects within the healthcare facility that are within UWB communication range of a patient support apparatus 20 and/or a locator unit 60. From this information, patient support apparatus server 84 is able to determine which objects are positioned closest to any patient support apparatus 20 at any given time.

It will also be understood that patient support apparatus server 84, in some embodiments, may also receive an utilize additional position information about the current locations of badges 142. That is, in addition to the position information of badges 142 as determined by their UWB communications with locator units 60 and/or with patient support apparatuses 20, patient support apparatus server 84 may also receive and utilize position information of badges 142 that is determined by badge server 94 using conventional WiFi location methods (e.g. triangulation, signal strength measurements, etc.). In such embodiments, patient support apparatus server 84 is configured to determine the location of badges 142 using two independent location-determination systems: a UWB-based location system and a WiFi-based location determination system. The locations determined from both of these systems may be combined by patient support apparatus server 84 to determine the location of badges 142 with better accuracy and/or with a broader range than when using only a single one of these location systems.

In some embodiments, controller 140 and/or patient support apparatus server 84 are adapted to use the position information of various objects and/or the association information of these various objects to carry out one or more automated tasks. These automated tasks include any one or more of the following: displaying cleaning instructions for cleaning personnel on display 52 and/or an associated display device 56; displaying a reminder to zero scale system 144 of patient support apparatus 20; determining when patient support apparatus 20 has been cleaned; determining when patient support apparatus 20 is ready to be cleaned; certifying that patient support apparatus 20 has been cleaned; determining the closest available transport worker for transporting a patient to another location; and/or identifying objects added to patient support apparatus 20, including the identity of the healthcare worker who added the objects. Each of these tasks will now be described in greater detail.

In some embodiments, controller 140 and/or patient support apparatus server 84 are configured to automatically determine if patient support apparatus 20 has been cleaned based upon the detection of one or more cleaning persons (with badges 142) within a volume of space 152c (or another volume of space) for more than a predetermined amount of time. That is, as was noted previously, controller 140 and/or patient support apparatus server 84 are configured to automatically determine the class of worker associated with any badge 142 that comes within a specified volume of space around patient support apparatus 20, such as volume of space 152c. If controller 140 and/or patient support apparatus server 84 detect that a badge 142 that is within the volume of space corresponds to a cleaning worker, one or both of them are configured to start a timer. The timer remains running until either the cleaning worker's badge 142 exits the volume of space 152c and/or the predetermined amount of time passes. If the badge 142 remains within the volume of space 152c for the predetermined amount of time, or longer, controller 140 and/or patient support apparatus 20 are configured to conclude that the patient support apparatus 20 has been cleaned.

In some embodiments, controller 140 and/or patient support apparatus server 84 are configured to analyze additional conditions before concluding that patient support apparatus 20 has been cleaned. One such additional condition is the presence of the patient's weight, as detected by scale system 144. If scale system 144 detects that the patient's weight is still present onboard patient support apparatus 20 while the healthcare worker's badge 142 remains within space volume of 152c, controller 140 and/or patient support apparatus server 84 may be configured to delay the start of the timer until the patient exits patient support apparatus 20 (as detected by scale system 144). In this manner, controller 140 and/or patient support apparatus server 84 will not conclude that a patient support apparatus 20 has been cleaned while a patient is present thereon, which is in accordance with the cleaning procedures of most healthcare facilities. That is, the proper cleaning of patient support apparatus 20 require disinfecting various patient support apparatus 20 while no patient is present thereon.

In some embodiments, controller 140 and/or patient support apparatus server 84 are configured to monitor the outputs of the force sensors of scale system 144 during the time the healthcare worker's badge 142 is present inside of volume of space 152c. If the force sensors do not detect a certain minimum amount of force and/or minimum amount of disturbances (which may be measured in the number of disturbances, the amplitude of the disturbances, and/or the duration of the disturbances), controller 140 and/or patient support apparats server 84 will not conclude that patient support apparatus 20 has been cleaned, even if the healthcare worker's badge 142 has remained inside volume of space 152c for the predetermined minimum amount of time (discussed above). The minimum amount of disturbances may be set based upon the normal amount of disturbances that would be expected for a thorough cleaning of patient support apparatus 20. Such “normal” disturbance are caused by the healthcare worker pressing the requisite cleaning utensils against the various surfaces of patient support apparatus 20 during the cleaning process.

Another condition that controller 140 and/or patient support apparatus 20 may consider when determining whether a patient support apparatus 20 is clean or not is the outputs of any sensors onboard patient support apparatus 20 that are adapted to detect cleaning agents. For example, in some embodiments, patient support apparatus 20 may include one or more volatile organic compound sensors, or other types of sensors, that detect the presence of cleaning compounds. In such embodiments, controller 140 and/or patient support apparatus server 84 are configured to not conclude that a patient support apparatus 20 has been cleaned unless such sensors detect the present of a cleaning chemical compound for a minimum amount of time and/or at a minimum concentration. In some embodiments, controller 140 and/or patient support apparatus server 84 are configured to conclude that a patient support apparatus 20 has been cleaned only if, in addition to the cleaning worker's badge 142 being positioned inside space volume 152c for the minimum amount of time, the patient support apparatus 20 detects a cleaner, or detects an aspect of the cleaning process, in any of the same manners as is disclosed in commonly assigned U.S. Pat. No. 10,492,967 issued Dec. 3, 2019, to inventor Marko Kostic and entitled SYSTEMS AND METHODS FOR DETERMINING THE USABILITY OF PERSON SUPPORT APPARATUSES, the complete disclosure of which is incorporated herein by reference.

Controller 140 and/or patient support apparatus server 84 may also be configured to consider still other conditions when automatically determining whether a patient support apparatus 20 has been cleaned, such as, but not limited to, the location of the patient support apparatus 20 within the healthcare facility, information from ADT server 102 (e.g. whether a patient is currently assigned to the room and/or bay of the patient support apparatus), and/or other information.

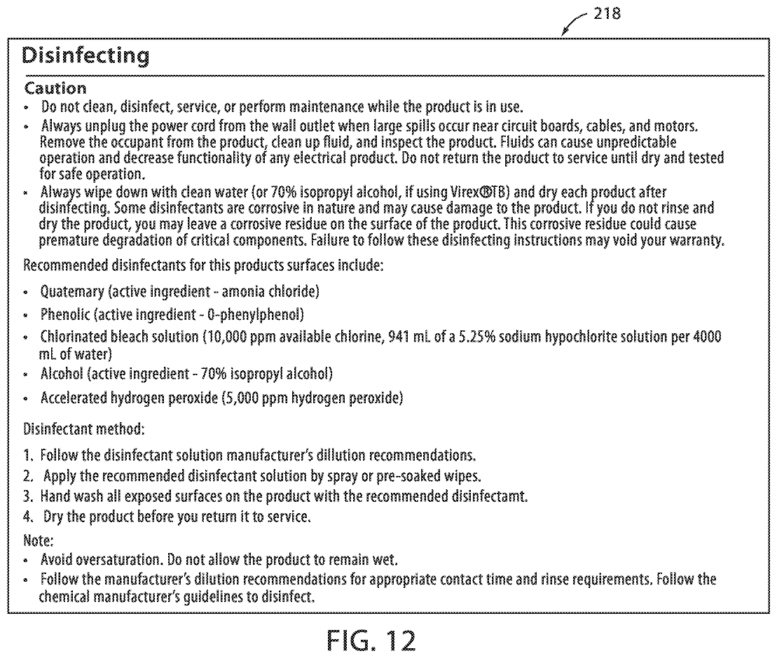

Another automated task that controller 140 and/or patient support apparatus server 84 may be configured to perform is the automatic display of cleaning instructions on display 52 of patient support apparatus 20 (or on an associated display device 56) in response to detecting a cleaning worker's badge 142 within volume of space 152c. One example of such cleaning instructions is shown in cleaning screen 218 (FIG. 12). Controller 140 and/or patient support apparatus server 84 are configured to automatically display cleaning screen 218 on display 52 and/or an associated display device 56 in response to detecting a cleaning worker's badge 142 within space volume 152c. In some embodiments, controller 140 and/or patient support apparatus server 84 may consider one or more additional conditions before automatically displaying a cleaning screen, such as cleaning screen 218. Such additional conditions may include the absence of the patient's weight on patient support apparatus 20 (as detected by scale system 144), the absence of a non-cleaning caregiver's badge 142 within space volume 152c, and/or any of the other conditions discussed above with respect to the automatic determination that patient support apparatus 20 has been cleaned.

Controller 140 and/or patient support apparatus server 84 may be configured to display cleaning instructions, such as cleaning screen 218 of FIG. 12, for a predetermined amount of time, such as for the entire time a cleaning worker's badge 142 is positioned within space volume 152c, or for a shorter, and in some cases, predetermined amount of time. Regardless of the amount of time controller 140 is configured to display screen 218, controller 140 may also be configured to allow the cleaning worker to navigate to other screens on display 52 and/or display device 56. In some embodiments, the cleaning instructions may be broken up into multiple screens 218 such that cleaning worker has to navigate to the other screens to see all of the cleaning instructions.

Another task controller 140 and/or patient support apparatus server 84 may be configured to automatically undertake is the identification of the cleaning worker, or workers, who completed the cleaning of a particular patient support apparatus 20. Controller 140 and/or patient support apparatus server 84 utilizes the badge ID 166 to determine whether a worker is a cleaning worker or not, as described previously. In addition to indicating the type of worker, badge ID 166 also contains information that allows controller 140 and/or patient support apparatus server 84 to determine the individual identity of that particular healthcare worker. Once controller 140 and/or patient support apparatus server 84 know the identity of the cleaning worker who cleaned a patient support apparatus 20 (based on the presence of the cleaning worker's badge 142 within space volume 152c for a minimum amount of time, and/or based on any of the previously mentioned additional conditions), controller 140 and/or patient support apparatus server 84 are configured to record this cleaning worker's identity. In some embodiments, controller 140 automatically displays the cleaning worker's identity on display 52 of patient support apparatus 20. In some such embodiments, controller 140 may also display other information about the cleaning, such as the time at which the cleaning took place.

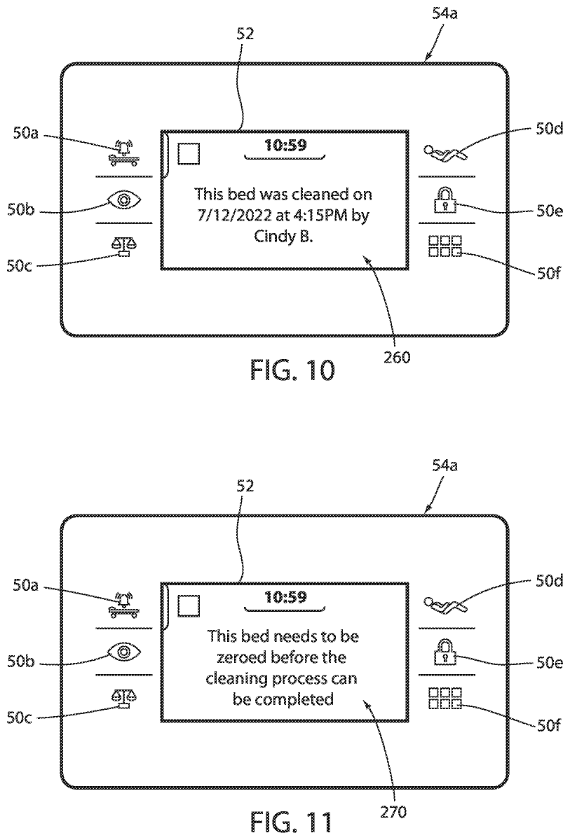

One example of the type of information about the cleaning process that a worker completed is shown in FIG. 10. FIG. 10 shows a cleaning identification screen 260 that identifies a particular cleaning worker (Cindy B.) who cleaned the patient support apparatus 20. Cleaning identification screen 260 also identifies the time and date at which the patient support apparatus 20 was cleaned. In some embodiments, controller 140 is configured to display cleaning identification screen 260 until a healthcare worker navigates to a “new patient” screen in which information regarding a new patient is entered into patient support apparatus 20, and/or until scale system 144 detects a weight of a patient onboard patient support apparatus 20. In this manner, patient support apparatus 20 continues to inform personnel of who cleaned it (and when) until a caregiver starts to use patient support apparatus 20 with a new patient.

Another task controller 140 and/or patient support apparatus server 84 may be configured to automatically undertake is the automatic reminding of healthcare workers to re-zero scale system 144 during, or after, the cleaning of patient support apparatus 20. Scale system 144, in some embodiments, is adapted to detect the weight of everything that is supported on litter frame 28, including the weight of litter frame 28, support deck 30, mattress 42, bedding, etc. In order to exclude the weight of those structures from the weight of the patient supported thereon, healthcare workers “zero” the scale prior to the patient getting into patient support apparatus 20. This zeroing process accounts for the weight of these structures so that the patient's weight can be separated from them. The zeroing process therefore accounts for the tare weight detected by scale system 144. Many healthcare facilities have guideline whereby the scale system 144 is to be re-zeroed before a new patient is assigned to patient support apparatus 20. Controller 140 and/or server 84 are configured to provide automatic reminders to the healthcare workers to zero scale system 144.

One example of a reminder to zero scale system 144 is shown in FIG. 11. FIG. 11 illustrates a zeroing reminder screen 270 that reminds the healthcare worker to zero patient support apparatus 20 as part of the cleaning process. Controller 140 is configured to automatically display screen 270 based on one or more factors. In some embodiments, controller 140 automatically displays screen 270 a predetermined amount of time after the presence of a cleaning worker's badge 142 is detected inside of space volume 152c (as detected by UWB transceivers 132). For example, if the average cleaning time of patient support apparatus 20 is a half hour, controller 140 may be adapted to automatically display screen 270 twenty to twenty-five minutes after the cleaning worker's presence is detected inside of space volume 152b, although other amounts of time may be used. Generally speaking, the zeroing process is completed at the end of, or after, the cleaning process, so it may be beneficial to delay displaying screen 270 until a point in time when the cleaning process is nearly done. In those instances where controller 140 is configured to automatically display cleaning instructions 218 (FIG. 12), controller 140 may be configured to automatically switch from displaying cleaning instruction screen 218 to displaying zeroing reminder screen 270 after the predetermined amount of time passes.

In some embodiments, instead of measuring a predetermined amount of time from the moment a cleaning worker's badge 142 is detected inside of space volume 152c, controller 140 may be configured to measure a predetermined amount of time from the moment controller 140 and/or patient support apparatus server 84 concludes that patient support apparatus 20 has been cleaned. After this amount of time passes, controller 140 may then automatically display screen 270, or a screen like it. Controller 140 is further configured to continue to display screen 270 until scale system 144 is zeroed. In some embodiments, controller 140 allows a healthcare worker to navigate to other screens, but controller 140 always returns to displaying screen 270 if control panel 54a is inactive for a defined amount of time and scale system 144 remains unzeroed. Once controller 140 detects that scale system 144 has been zeroed, it automatically stops displaying zeroing reminder screen 270 (until the next cleaning cycle is detected).

In some embodiments, controller 140 may be configured to automatically send a reminder message to the cleaning worker's badge 142 to remind him or her of the task of zeroing scale system 144. This reminder message may be sent in addition to, or in lieu of, the display of zeroing reminder screen 270. In such embodiments, controller 140 may be configured to send the reminder message a predetermined amount of time after the presence of the cleaning worker's badge 142 is detected inside space volume 152c, a predetermined amount of time after the patient support apparatus 20 has been determined to be cleaned, and/or at other times. In some embodiments, controller 140 is configured to send the reminder message if the cleaning worker's badge 142 moves outside of volume of space 152c without scale system 144 having been zeroed.

The reminder message to the cleaning worker's badge may be sent in a different manners. In some embodiments, controller 140 uses one or more of the UWB transceivers 132 to send the message directly to the UWB transceiver of the cleaning worker's badge 142. In some embodiments, if the cleaning worker's badge 142 includes a Bluetooth transceiver, controller 140 may send the reminder message directly to the healthcare worker's badge 142 using Bluetooth transceiver 128. In still other embodiments, controller 140 may send a message to badge server 94 via network transceiver 96, and badge server 94 may then relay the message to the cleaning worker's badge via WiFi using access points 82 and the badge's network transceiver 164.

Another task controller 140 and/or patient support apparatus server 84 may be configured to undertake is the automatic determination of when a patient support apparatus 20 is ready to be cleaned. In such embodiments, controller 140 and/or patient support apparatus server 84 are configured to take into account one or more conditions for making the automatic determination that a patient support apparatus 20 is ready to be cleaned. In one such embodiment, controller 140 and/or patient support apparatus server 84 are configured to conclude that patient support apparatus 20 is ready to be cleaned after the presence of a transport worker's badge 142 is detected inside of volume of space 152c. Such transport workers are often used to transport a worker to an exit of the healthcare facility after the patient has been discharged from the healthcare facility. In such embodiments, controller 140 and/or patient support apparatus server 84 are configured to conclude that patient support apparatus 20 is ready to be cleaned after the presence of the transport worker's badge 142 is initially detected inside of space volume 152c and then is subsequently detected exiting space volume 152c. The exiting of the badge 142 from space volume 152c is presumed to occur while the patient is with the transport worker, thereby establishing that patient support apparatus 20 is no longer being used by that particular patient, and therefore is ready to be cleaned.

In some embodiments, controller 140 and/or patient support apparatus server 84 are configured to consider other conditions prior to concluding that a patient support apparatus 20 is ready to be cleaned. For example, in some embodiments, controller 140 is further configured to detect a UWB tag 188 worn by the patient, and controller 140 and/or patient support apparatus server 84 are configured to conclude that the patient support apparatus 20 is ready to be cleaned only if both of the following conditions are met: (1) the transport worker's badge 142 is initially detected inside space volume 152c and then moves outside of this space volume 152c; and (2) the UWB tag 188 worn by the patient also exits from space volume 152c around the same time as the transport worker's badge 142.

In some embodiments, controller 140 and/or patient support apparatus server 84 utilize still other conditions for determining whether patient support apparatus 20 is ready to be cleaned. For example, in some embodiments, patient support apparatus server 84 is configured to receive data from ADT server 102 indicating when a patient has been discharged. In such embodiments, controller 140 and/or patient support apparatus server 84 may be configured to not conclude that a particular patient support apparatus 20 is ready to be cleaned until the patient who was assigned to that particular patient support apparatus 20 has been discharged, as indicated by data stored in ADT server 102.

Another condition that controller 140 and/or patient support apparatus server 84 may utilize is the patient weight, as detected by scale system 144. In such embodiments, controller 140 and/or patient support apparatus server 84 are configured to monitor the patient's weight on patient support apparatus 20, as detected by scale system 144, and to not conclude that the patient support apparatus 20 is ready to be cleaned unless the patient's weight is no longer detected on patient support apparatus 20.

The aforementioned conditions that controller 140 and/or patient support apparatus server 84 may utilize when determining whether a particular patient support apparatus 20 is ready to be cleaned may be combined in different manners in different embodiments. For example, in one embodiment, controller 140 and/or patient support apparatus server 84 conclude that a particular patient support apparatus 20 is ready to be cleaned after the following three conditions are met: (1) the transport worker's badge 142 is initially detected inside space volume 152c and then moves outside of this space volume 152c; (2) the UWB tag 188 worn by the patient also exits from space volume 152c around the same time as the transport worker's badge 142; and (3) ADT server 102 indicates that the patient has been discharged from the healthcare facility. As another example, controller 140 and/or patient support apparatus server 84 may be configured to substitute, or supplement, condition (2) mentioned above with the condition that the patient's weight has to be detected as being absent from the particular patient support apparatus 20 prior to controller 140 and/or patient support apparatus server 84 concluding that the patient support apparatus is ready to be cleaned. In other words, either the patient's tag 188 has to be detected outside of space volume 152c or the patient's weight has to be absent from patient support apparatus 20 before condition (2) is satisfied. Still other combinations of conditions may be required by controller 140 and/or patient support apparatus server 84 before one of them concludes that patient support apparatus 20 is ready to be cleaned.

Once controller 140 and/or patient support apparatus server 84 has determined that a particular patient support apparatus 20 is ready to be cleaned, either or both of them may be configured to automatically send a message to one or more cleaning workers and/or a cleaning server (not shown) on local network 80. The message informs the cleaning worker that the particular patient support apparatus 20 is ready to be cleaned so that they can add it to their work assignment. In some embodiments, the message is sent to the cleaning worker's badge 142 using badge server 94. In other embodiments, patient support apparatus server 84 may be configured to send the message directly to the cleaning worker's badge 142 without using badge server 94. In still other embodiments, the message may be sent to a cleaning server that the cleaning workers may be able to log into, or otherwise access, (such as by using their electronic devices 98) to see a list of patient support apparatuses 20 that are ready to be cleaned.

Controller 140 and/or patient support apparatus server 84, in some embodiments, are configured to automatically determine the nearest badge 142 to different locations within the healthcare facility. In some embodiments, controller 140 and/or patient support apparatus server 84 determine the nearest badges for multiple classes of workers. In other words, they determine the nearest caregiver, the nearest transport worker, the nearest cleaning worker, the nearest doctor, etc. In some embodiments, controller 140 and/or patient support apparatus server 84 are configured to automatically determine the nearest badge 142 assigned to a transport worker when a patient is ready to be discharged and/or transported to a different location within the healthcare facility. In such embodiments, controller 140 and/or patient support apparatus server 84 receive data from ADT server 102 indicating when a particular patient is discharged, or scheduled to be transported to another location within the healthcare facility. In response to receipt of this data, controller 140 and/or patient support apparatus server 84 may automatically send a message to a transport worker's badge 142 that is positioned closest to the patient support apparatus 20 assigned to that particular patient. The message informs the transport worker that the particular patient needs to be transported to a different location within the healthcare facility.

In some embodiments, controller 140 and/or patient support apparatus server 84 may take into account the workload of the transport workers, and if the nearest transport worker's badge 142 is assigned to a particular transport worker who has a list of assignments that exceed a threshold, controller 140 and/or patient support apparatus server 84 may then determine who the second nearest transport worker is to the patient support apparatus 20 of the particular patient. If that second nearest worker is already working on a transportation assignment, or if his or her list of assignments is over a threshold and a different transport worker has a smaller list of assignments, controller 140 and/or patient support apparatus server 84 may then find the third nearest transport worker, and so on. Once controller 140 and/or patient support apparatus server 84 has identified the closest transport worker with the best workload for handling the transport of the particular patient, controller 140 and/or patient support apparatus server 84 send a message to that transport worker's badge 142 (or to a transportation server on network 80) informing the transport worker of this new patient-transportation assignment.

As was discussed previously, when controller 140 and/or patient support apparatus server 84 determine the nearest particular badge 142 to a particular location within the healthcare facility, they may utilize the location information of badges 142 as determined from the UWB transceivers 132 onboard patient support apparatuses 20, from the UWB transceivers 126 and 126a onboard locator units 60, and also from the WiFi location information that badge server 94 may be configured to gather about badges 142. Alternatively, or additionally, this location information may be supplemented by a conventional real-time location system that includes a server on local network 80 that is able to share worker position information with controller 140 and/or patient support apparatus server 84.

Another task that controller 140 and/or patient support apparatus server 84 may be configured to automatically implement is the detection and/or recordation of non-patient weights that are added to patient support apparatus 20. Patient support apparatus 20, in some embodiments, is configured to maintain a equipment weight log that identifies what non-patient weights have been added to, and removed from, patient support apparatus 20 subsequent to the zeroing of scale system 144. Controller 140 uses the equipment weight log to determine what portion of the non-tared weight supported on patient support apparatus 20 belongs to the patient versus what portion belongs to equipment, or other non-patient objects. In some prior art patient support apparatuses 20, the updating of this equipment weight log is carried out manually by a user informing the patient support apparatus, such as through control panel 54a, when a non-patient weight is being added or removed from patient support apparatus 20.

In one of the embodiments disclosed herein, controller 140 is configured to automatically determine whether a non-patient weight has been added to patient support apparatus 20 by monitoring the position of the weight using UWB transceivers 132. The non-patient weight therefore can be the result of a device 100 or an object with a tag 188 being added to patient support apparatus 20. By determining the three-dimensional relative position of the device 100 or tag 188, along with knowing the three-dimensional position of the support deck 30, litter frame 28 and siderails 36 on patient support apparatus 20, controller 140 is configured to determine if the device 100 or tag 188 is supported on patient support apparatus 20 or not. In some embodiments, controller 140 also monitors the outputs from scale system 144 to determine if the device 100 or tagged object is positioned on patient support apparatus 20. If no additional weight is detected, the controller 140 concludes that the device 100 or tagged object has not been added to patient support apparatus 20. If an additional weight is detected, controller 140 concludes that a device 100 tagged object has been added.

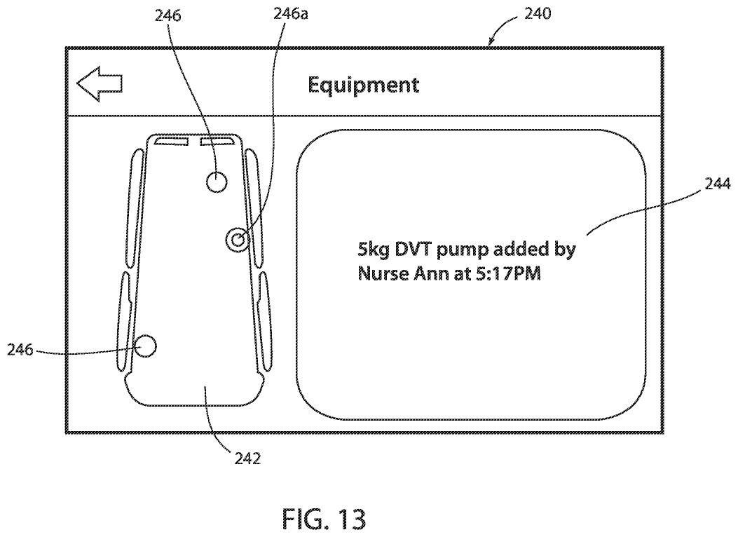

Controller 140, in some embodiments, is configured to display one or more screens on display 52 that provide information about the non-patient objects that are currently contained within the equipment weight log. Weight log screen 240 of FIG. 13 provides one example of such a screen. Weight log screen 240 includes a patient support apparatus icon 242, a text area 244, and, depending upon the current number of objects positioned on patient support apparatus 20, one or more weight indicators 246. Patient support apparatus icon 242 is a graphical representation of patient support apparatus 20 taken from a point of view above patient support apparatus 20. Icon 242 therefore illustrates the top surface of patient support apparatus 20 on which a patient is intended to sit or lie. Weight indicators 246 are positioned on icon 242 at locations where an object has been added to patient support apparatus 20. Controller 140, in some embodiments, is adapted to determine the locations of the added objects based on the UWB-determined location of these objects and, in some embodiments, based on a calculation of the center of gravity of these objects using scale system 144. In addition to the location of the objects added to patient support apparatus 20, controller 140 is further configured to determine which healthcare added the these weights to the patient support apparatus 20, as well as the time at which these weights were added.

Controller 140 determines which healthcare worker added the weights corresponding to weight indicators 246 by determining which healthcare worker's badge 142 was present within a volume of space, such as volume of space 152c and/or 152b, when the object was first added to patient support apparatus 20. As explained elsewhere herein, controller 140 may use the badge identifier 166, either alone or in conjunction with patient support apparatus server 84, to determine the identity of the caregiver associated with that particular badge 142. Controller 140 may then display that caregiver's identity in message area 244 when the user selects one of the weight indicators 246. For example, in the situation illustrated in FIG. 13, a user has selected weight icon 246a and controller 140 is displaying information in message area 244 that correspond to weight icon 246a. As shown therein, the weight icon 246a corresponds to a five kilogram DTV pump that was added at 5:17 PM by Nurse Ann.

From message area 244 of FIG. 13, it can be seen that controller 140 may also be configured to not only identify where an object was added to patient support apparatus 20, its location on patient support apparatus 20, the time at which it was added, and the person responsible for adding it, but also my determine an identification of the object. In some embodiments, controller determines an identity of the object (e.g. a DVT pump) by using its identifier 166 and/or 180 and a look-up table that correlates identities to specific types of devices. The look-up table may reside on patient support apparatus 20 and/or it may reside on patient support apparatus server 84.

FIG. 8 illustrates an overview of the communication system of the present disclosure, including various types of data that may be stored within different servers on a healthcare facility's computer network 80. As was noted previously, EMR server 92 stores the electronic medical records of individual patients, and this data may include individual care plans for each patient, such as shown in table 322. ADT server 102 stores data indicating the current location within the healthcare facility for each patient, such as is indicated in table 320. Badge server 94 stores badge IDs (e.g. identifiers 180), the healthcare workers associated with those badge IDs, and whether or not the healthcare worker is currently logged into the system (e.g. whether the worker is currently using the system or not), as indicated by table 326. Caregiver assignment server 104 stores the rooms—and/or bays within rooms—of the patients that each caregiver is assigned to care for, as shown in table 324. Patient support apparatus server 84 stores a variety of data, some of which is illustrated in patient support table 330 of FIG. 8.

Patient support table 330 includes a column of locator IDs 122, a column of rooms, a column of bed status information, a column indicating the presence or absence of caregivers, a column indicating what devices 100 are associated with each patient support apparatus 20, a column of patient support apparatus IDs 130, and a column of function identifiers that indicate what functions have been performed using each patient support apparatus 20. The locator IDs 122, 122a are forwarded by each patient support apparatus 20 to patient support apparatus server 84 after patient support apparatus 20 associates itself with a particular locator unit 60. The rooms or locations corresponding to each locator ID 122, 122a are determined automatically by patient support apparatus server 84 using its onboard memory, which includes the surveying information from the installation of the locator units 60 (which, in turn, identifies the rooms and/or bays of each locator unit 60).

Patient support apparatus server 84 determines whether a healthcare worker is currently positioned nearby to each patient support apparatus 20 (or unlinked locator unit 60a) based upon whether the healthcare worker's badge 142 is currently associated with that patient support apparatus 20 or not (or with that unlinked locator unit 60a or not). In other words, when controller 140 associates patient support apparatus 20 with a badge 142, it forwards this association determination (and the badge ID 180) to patient support apparatus server 84, which uses the ID 180 to determine the caregiver's identity. As long as the badge 142 remains associated with patient support apparatus 20, patient support apparatus server 84 considers the caregiver to be present within the vicinity of the patient support apparatus 20. The same is true for badges 142 and unlinked locator units 60a. Once controller 140 disassociates a badge 142 from patient support apparatus 20, it also sends a message to patient support apparatus server 84 and patient support apparatus server 84 then concludes that the caregiver is no longer present within the vicinity of the patient support apparatus 20. Similarly, once a badge 142 is disassociated from an unlinked locator unit 60a, the badge 142 and/or the unlinked locator unit 60a sends a message to patient support apparatus server 84 and patient support apparatus server 84 then concludes that the caregiver is no longer present within the vicinity of that unlinked locator unit 60a.

As shown in table 330 of FIG. 8, patient support apparatus server 84 also receives data from the objects (devices 100, tags 188, and/or badges 142) that are currently associated with each patient support apparatus 20, as shown in the column labeled “Assoc. Device Status.” As has been described herein, patient support apparatus server 84 may forward some or all of this data to EMR server 92, one or more electronic devices 98, one or more other devices, and/or store this data. Table 330 also includes the patient support apparatus IDs 130 of each patient support apparatus 20. Still further, table 330 includes a column containing data identifying what functions have been performed with each patient support apparatus 20. This functional data is sent by controller 140 to patient support apparatus server 84 in response to a user carrying out one or more functions of patient support apparatus 20, as was descried previously.

It will be understood that EMR server 92, badge server 94, caregiver assignment server 104, and ADT server 102 (FIGS. 4 and 8) may all be conventional and/or commercially available servers.

In some embodiments, when patient support apparatus 20 and/or server 84 associate a device 100, tag 188, and/or badge 142 with a particular patient support apparatus 20, controller 140 and/or server 84 are configured to inform medical personnel (via electronic devices 98) that the device, tag, or badge is associated with a particular patient support apparatus 20 and/or with a particular patient assigned to that patient support apparatus 20. In this manner, data from the device, tag, and/or badge can be correlated with a particular patient, room location, and/or patient support apparatus 20. Patient support apparatus 20 may therefore be configured to automatically forward data from the associated device, tag, and/or badge to server 84 after these objects become associated with patient support apparatus 20. Some of such data may, in turn, be automatically forwarded by server 84 to EMR server 92 for entry into the corresponding patient's electronic medical record. In this manner, once controller 140 associates a device, tag, and/or badge 142 with the patient support apparatus 20, data from the device, tag, and/or badge can be automatically recorded in that patient's particular electronic medical record without requiring the caregiver to either manually associate these objects with the patient and/or with the patient support apparatus 20 assigned to that patient. In other words, because controller 140 automatically determines that the device, tag, or object is positioned within a corresponding space volume 152, it is not necessary for a caregiver to take any manual steps to ensure that data from the objects is forwarded to the proper corresponding patient's electronic medical record because patient support apparatus 20, along with server 84, automatically determine the correct patient associated with that device, tag, or badge.

In those situations where controller 140 and/or server 84 determine the identity of a specific patient assigned to a particular patient support apparatus, controller 140 and/or server 84 communicates with ADT server 102 (FIG. 8). ADT server keeps track of which patients are assigned to which rooms and/or which bays of rooms with multiple patients. In order for controller 140 or server 84 to determine which patient is assigned to a particular patient support apparatus 20, it needs only to determine which room and/or bay that particular patient support apparatus 20 is currently located in. This is accomplished through communications with an associated locator unit 60, as discussed above. In brief overview, controller 140 either sends the locator unit ID 122 of an associated locator unit 60 to patient support apparatus server 84, which then looks up the room and/or bay location of that locator unit ID 122, or controller 140 consults an onboard listing of what rooms and/or bays correspond to each particular locator unit 60. In either case, the room or bay number of a particular patient support apparatus 20 is determined. After determining which room or bay a particular patient support apparatus is located in, ADT server 102 is consulted to determine the particular patient assigned to that particular room or bay (and thus that particular patient support apparatus 20).

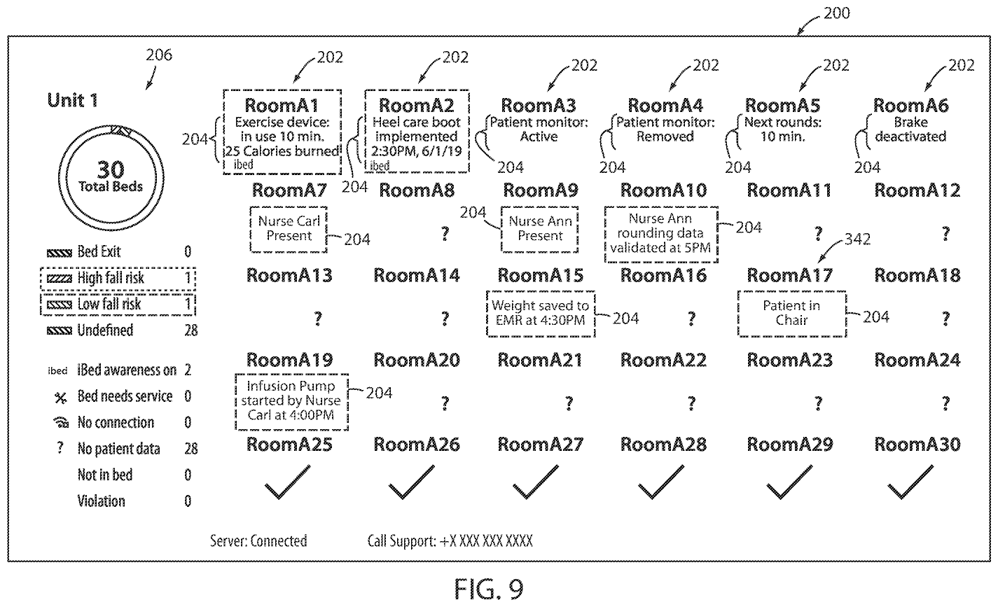

FIG. 9 illustrates one example of the type of data that patient support apparatus 20 may be configured to send to one or more electronic devices 98, either directly, through patient support apparatus server 84, or through other intermediaries. More specifically, FIG. 9 illustrates one example of a dashboard screen 200 that patient support apparatus server 84 may be configured to display on one or more electronic devices 98. Dashboard screen 200 is a multi-room dashboard display that indicates the status of patient support apparatuses 20 in a plurality of rooms of a healthcare facility, as well as information regarding the location and activities of healthcare workers with badges 142. The electronic devices 98 on which dashboard screen 200 may be displayed include WiFi connections for communicating with access points 82 of network 80, and thus are able to receive status data from patient support apparatus server 84.

Dashboard screen 200 includes a plurality of individual room numbers 202 and corresponding status messages 204 indicating one or more parameters about the status of the patient assigned to that room, the patient support apparatus 20 in that room, the caregiver assigned to the patient in that room, and/or any other workers with badges 142 who may be present in the room and/or who may have performed one or more functions on that patient support apparatus 20. The information that is displayable on screen 200 includes any information regarding the patient support apparatus 20 positioned in that room, as well as any information about badges 142, devices 100, tags 188, and/or locator units 60 that are associated with that patient support apparatus. Thus, for example, as shown in FIG. 9, status messages 204 may indicate that an exercise device 100 has been in use for ten minutes and the patient has burned 25 calories (room A1), that a heel care boot 100 has been applied to a patient as of 2:30 PM on Jun. 1, 2019 (room A2); that a patient monitor 100 is active (room A3); that a patient monitor has been moved out of proximity of a patient support apparatus 20 (e.g. out of space volume 152b) (room A4); that a caregiver is expected to perform a rounding duty for a patient within a specific time period (room A5); that a brake on patient support apparatus 20 is deactivated (room A6); that nurse Carl is currently present in rooms A7 (as determined through the detection by UWB transceivers 132 of his badge 142); that the bed in room A8 needs to be zeroed (as determined in any of the manners previously discussed herein); that Nurse Ann is currently present in room A9; that patient N. Johnson arrived in room A10 at 4:30 (as determined through communication with ADT server 102, through the detection of the patient's tag 188 within a space 152b or c, and/or through the detection of a transport workers' badge 142 within that space); that a weight log of the patient support apparatus 20 located in room A11 was updated by Nurse Ann at 11:00 PM; that patient M. Smith has been discharged from room A13 and is awaiting transportation; that the patient's weight in room A15 was sent to EMR server 92 at 4:30 PM; that the patient C. Miller is currently sitting in a recliner in room A17 (where the recliner includes a patient presence sensor and is a UWB-equipped device 100); and that an infusion pump was started by Nurse Carl at 4 PM in room A19, and that the bed in room A21 is ready to be cleaned. Still other types of information may be displayed on dashboard screen 200, including additional details regarding the status messages 204. Such additional details may be displayed in response to, for example, a user tapping on, or otherwise selecting a particular one of the rooms 202 on screen 200. All of the data shown in the various rooms of FIG. 9 is generated in accordance with the principles discussed here.

Screen 200 may also include a summary status area 206 that lists certain collective data statistics about patient support apparatuses 20, devices 100, badges 142, and/or patients. For example, summary area 206 may indicate how many patient support apparatuses 20 are currently issuing an exit alert (detected by exit detection system 136); how many patients are classified as high or low fall risks (or whose fall risk categorization has not been completed); how many patient support apparatuses 20 have their onboard monitoring system 138 activated; how many patient support apparatuses 20 are in need of service; how many are not connected to network 80, and/or other data. As noted, tapping on, or otherwise selecting, any individual room 202 of screen 200 may cause the electronic device 98 to display additional information about the status of the patient, patient support apparatus 20, and/or any device(s) 100 and/or badges 142 that are positioned in that room.

In some embodiments, controller 140 is configured to send messages to patient support apparatus server 84 indicating one or more functions that have been performed on patient support apparatus 20. The content of these messages may be displayed on dashboard screen 200 and/or it may be saved in a memory accessible to server 84. Examples of the type of functions that controller 140 is configured to notify patient support apparatus server 84 of include the following: changing a state of a brake on patient support apparatus 20; changing a height of litter frame 28; changing an angle of head section 44; making other changes to the angle, position, and/or configuration of litter frame 28 and/or support deck 30; raising or lowering siderails 36; arming or disarming exit detection system 136; arming or disarming monitoring system 138; weighing a patient using scale system 144; zeroing, or making other changes to, scale system 144; adding or removing items from an equipment weight log maintained by patient support apparatus 20; deleting patient data stored in memory 134; entering new patient information into memory 134; locking or unlocking any one or more controls of patient support apparatus 20; power cycling patient support apparatus 20; plugging in or unplugging AC cord 150 into or out of an electrical outlet 108; plugging in or unplugging nurse call cable 66 into or out of cable port 148 and/or communication outlet 64; moving patient support apparatus 20 to a different location (as detected through the connection/disconnection of patient support apparatus 20 from multiple locator units 60); adjusting the inflation state of mattress 42; implementing a therapy and/or a patient turn using inflatable mattress 42; changing a setting of patient support apparatus 20; and/or other changes.

Each of these functions is carried out by manipulating one or more controls onboard patient support apparatus 20, and controller 140 is configured to assign a function identifier to each of these functions. Further, controller 140 is configured to forward these function identifiers to patient support apparatus server 84 after these functions are carried out. Patient support apparatus server 84 has a directory of function identifiers and the functions corresponding to these identifiers, which it uses to determine from the function identifier what functions have been performed on patient support apparatus 20. Patient support apparatus server 84 may then forward information about the functions that have been performed to one or more recipients, such as EMR server 92, one or more electronic devices 98, one or more computers on network 80 (e.g. computer 168; FIG. 8), and/or to other recipients. It may also, as noted, save data regarding these functions and/or display it on dashboard screen 200.

In some embodiments, controller 140 is configured to allow a user to customize what functions are transmitted to patient support apparatus server 84 for recordation, display, and/or forwarding. This customization may be carried out by an authorized user utilizing control panel 54c and/or by accessing patient support apparatus server 84. For example, controller 140 may be customized by an authorized user to not transmit any functions that are implemented on patient support apparatus 20 via the patient control panels 54c, but instead only transmit functions performed using caregiver controls panels 54a and/or 54c (and/or functions that don't require utilizing a control panel). As another example, controller 140 may be configured by an authorized user to not transmit changes to the positions of siderails 36, but to transmit changes to the brake, exit detection system 136, monitoring system 138, etc. Still other variations, of course, may be customized by authorized personnel.

Some functions that are completed by a user wearing a badge 142 may be desirably recorded in EMR server 92. For example, patient weight readings taken using scale system 144 may be desirably recorded in EMR server 92. Controller 140 is further customizable by the user as to which functions are to be recorded and automatically forwarded to EMR server 92. When controller 140 forwards these completed functions to EMR server 92, controller 140 also forwards caregiver identity information to EMR server 92 regarding the identity of the caregiver who implemented these functions. In some instances, the identity information includes the name and/or a unique identifier of the caregiver. Patient support apparatus server 84 determines the caregiver's name or unique identifier using the current location of patient support apparatus 20 and by receiving data from caregiver assignment server 104, which, as noted previously, may store the names of caregivers (or their unique IDs) and their corresponding assignments to specific rooms and/or bays.

Function data that is recorded on patient support apparatus 20 (e.g. in memory 134) and/or in patient support apparatus server 84 is subsequently retrievable by authorized personnel. Such data may be retrieved using a conventional computer, such as computer 168 (FIG. 8) that has access to network 80 and patient support apparatus server 84, by using an electronic device 98 that has access to network 80 and patient support apparatus server 84, and/or by one or more of the control panels 54 of patient support apparatus 20. By retrieving this information, authorized users can see not only the work performed by individual caregivers, but can also determine whether procedures, therapies, and/or treatments were completed in a proper and/or timely manner.

In some embodiments, after determining that the relative location of one or more of devices 100, tags 188, and/or badges 142 are currently positioned within a corresponding space volume 152, controller 140 forwards a message to patient support apparatus server 84 indicating that these objects (whose IDs are included in the message) are currently located in the same room as patient support apparatus 20. Patient support apparatus server 84 may display this information on dashboard screen 200 (FIG. 8) and/or forward this information to one or more of electronic devices 98, thereby enabling authorized individuals to track the location of pieces of equipment within the healthcare facility. Controller 140 may therefore, in addition to determining the location of UWB-equipped objects (devices 100, tags 188, and/or badges 142) for purposes of communicating data to/from those devices, also determine the location of those objects for tracking purposes. Patient support apparatus 20 therefore can facilitate the tracking of a variety of equipment used within the healthcare facility for inventory management purposes and/or for performing timely service on the equipment.

In some embodiments, UWB transceivers 126, 126a, 132, 158, and 178 (FIGS. 5 & 7) may operate in the same manner as, and include any of the same functions as, the anchors and pseudo-anchors disclosed in commonly assigned U.S. patent application Ser. No. 63/193,777 filed May 27, 2021, by inventors Thomas Deeds et al. and entitled SYSTEM FOR ASSOCIATING MEDICAL DEVICE DATA, the complete disclosure of which has already been incorporated herein by reference. In some embodiments, locator units 60 may also be configured to determine the location of a device (e.g. device 100, badge 142) in any of the manners disclosed in commonly assigned U.S. patent application Ser. No. 63/132,514 filed Dec. 31, 2020, by inventors Alexander Bodurka et al. and entitled PATIENT SUPPORT APPARATUS AND MEDICAL DEVICE NETWORKS, and in commonly assigned U.S. patent application Ser. No. 63/154,677 filed Feb. 27, 2021, by inventors Celso Pereira et al. and entitled SYSTEM FOR DETERMINING PATIENT SUPPORT APPARATUS AND MEDICAL DEVICE LOCATION, the complete disclosures of both of which are incorporated herein by reference.

It will also be understood that, although FIG. 5 illustrates badge 142 as including a UWB transceiver 158, patient support apparatus 20 and patient support apparatus server 84 may be configured to implement any of the functions described herein using badges 142 that do not include a UWB transceiver 158. In such situations, patient support apparatus server 84 determines whether to associate a badge 142 with a particular patient support apparatus 20 based upon the location information that may be independently gathered by badge server 94 using conventional WiFi triangulation techniques (or other conventional location determination techniques). Although the location technology utilized by badge server 94 may not be as precise as the UWB location technology described herein, patient support apparatus server 84 may be configured to use this location information to determine whether to associate and disassociate non-UWB equipped badges 142 with patient support apparatuses 20.

Although the foregoing description has primarily indicated that controller 140 determines whether to associate or disassociate patient support apparatus 20 with one or more objects (devices 100, badges 142, tags 188) and/or locator units 60, it will be understood that the association and/or disassociation process may alternatively, partially, and/or additionally, be carried out by patient support apparatus server 84. In such embodiments, controller 140 forwards relative position information regarding these objects to patient support apparatus server 84 so that server 84 can determine whether to associate a particular object with a particular patient support apparatus 20. In such embodiments, server 84 sends a message back to patient support apparatus 20 informing it of what objects it should be associated with, and what devices it should not be associated with.

In some embodiments, server 84 is configured to associate or disassociate tags 188 and/or devices 100 with specific patients. Server 84 may implement this patient association and disassociation process by storing in its memory data correlating specific device identifiers 166 or 180—which are transmitted to patient support apparatus 20 by their corresponding devices, and then forwarded by patient support apparatus 20 to server 84 via network transceiver 96—with specific patient support apparatus identifiers 130. In order to associate specific objects with a specific patient, patient support apparatus server 84 uses the location identifier 122 that it receives from a specific patient support apparatus 20 to determine the room location of the specific patient support apparatus 20. From this room location, server 84 receives data from ADT server 102 that identifies a specific patient for a specific room (or bay within a room). Server 84 is then able to correlate a specific device 100, tag 188, or badge 142 (associated with the specific patient support apparatus 20 in that room) with a specific patient identifier because it knows the room (or bay) of the patient support apparatus 20, the specific IDs of the objects associated with that particular patient support apparatus 20, and the patient identifier associated with that particular room (or bay). After associating am object with a particular patient, server 84 is able to automatically retrieve data from a specific patient's medical records stored within EMR server 92 and/or to automatically send data from a particular device to the specific patient's medical records stored in EMR server 92.

Although FIG. 5 illustrates a single device 100, a single tagged object 190, and a single badge 142, it will be understood that this is merely for illustration purposes. Multiple devices 100, object 190, and/or badges 142 may be within communication range of UWB transceivers 132 of patient support apparatus 20 and controller 140 of patient support apparatus 20 is adapted to determine the locations of those objects relative to a corresponding space volume 152b, c, d, etc. and, if they are positioned inside of the corresponding space volume 152, to automatically associate them with patient support apparatus 20 and make their data available for display, forward their data to patient support apparatus server 84, and/or take any of the other actions described herein.

In some embodiments, controller 140 is adapted to automatically associate itself with any one or more of the devices disclosed in commonly assigned U.S. patent application 63/154,677 filed Feb. 27, 2021, by inventors Celso Pereira et al. and entitled SYSTEM FOR DETERMINING PATIENT SUPPORT APPARATUS AND MEDICAL DEVICE LOCATION, the complete disclosure of which is incorporated herein by reference. Patient support apparatus 20 is configurable by a user to also, or alternatively, automatically display any of the data from these other types of devices on any of the displays 52 and/or associated display devices 56 when these devices are associated with patient support apparatus 20.

It will be understood that the frequency at which controller 140 repetitively determines the relative location of devices 100, tags 188, locator units 60, and badges 142 may vary in different embodiments. In some embodiments, this occurs multiple times a second. In other embodiments, this occurs less frequently than once a second. In still other embodiments, the frequency at which controller 140 determines the relative position of an object to patient support apparatus 20 may vary according to the object, the current location of patient support apparatus 20, the movement state of patient support apparatus 20 (i.e. whether it is currently moving or stationary), the brake state of patient support apparatus 20 (i.e. whether the brake is on or off), and/or according to one or more other factors.

Controller 140 is configured to use the association status of a linked locator unit 60 (FIG. 4) to determine where to send data to for forwarding to the adjacent communication outlet 64, as well as to determine which linked locator unit 60 it will accept data from that originated from communication outlet 64. The associated linked locator unit 60 is the linked locator unit 60 that patient support apparatus 20 will send the patient's voice signals to for forwarding to nurse call system 70. It is also the linked locator unit 60 that controller 140 will send television commands to when a patient onboard patient support apparatus 20 activates one or more of the television controls 50l-50r. Similarly, it is the linked locator unit 60 that controller 140 will send light commands to when a patient onboard patient support apparatus 20 activates one or more or the reading or room light controls 50s or 50t. The linked locator unit 60 that patient support apparatus 20 associates itself with is also the linked locator unit 60 that patient support apparatus 20 will receive audio signals from and direct to its onboard speaker(s). Such audio signals may correspond to voice signals from a remotely positioned nurse that are forwarded to the corresponding communication outlet 64 by way of nurse call system 70, or such audio signals may correspond to television audio signals that are routed from television 72 to communication outlet 64 by way of the one or more conductors 68.

In some embodiments, patient support apparatus 20 is configured to automatically zero scale system 144 when one or more conditions are satisfied, and to not automatically zero the scale system 144 when any one or more of these conditions are not satisfied. As will be discussed in greater detail below, controller 140 may be configured to require one or more of the following conditions before carrying out an automatic zeroing process of scale system 144: (1) controller 140 detects no patient tag 188 within a threshold distance of patient support apparatus 20; (2) controller 140 detects no caregiver badge 142 or tag 188 within a threshold distance of patient support apparatus 20; (3) a brake 250 of patient support apparatus 20 is currently activated; (4) an obstruction detection system 252 of patient support apparatus 20 does not currently detect an obstruction; (5) litter frame 28 is at a height above a threshold; (6) an electrical cord of patient support apparatus 20 is currently plugged into a power outlet; (7) the force sensors (e.g. load cells) of scale system 144 have not detected any disturbances above a first threshold for at least a second threshold amount of time; (8) the currently measured non-patient weight and non-logged equipment weight is greater than a threshold; and/or (9) other conditions.

With respect to the first potential condition, controller 140 may be configured to use UWB transceivers 132 to check for the presence of a patient tag 188 within a close vicinity of patient support apparatus 20 (such as, for example, about 1-3 meters, although other distances may be used). If no such patient-worn UWB tag 188 is found in this vicinity, controller 140 may conclude that patient support apparatus 20 is not currently occupied by a patient, and therefore this first condition for automatically zeroing scale system 144 is met. This first condition therefore serves the purpose of ensuring that not only is patient support apparatus 20 not occupied when a potential automatic-zeroing process takes place, but that it is unlikely that a patient will enter patient support apparatus 20 during the next few seconds when the automatic-zeroing process may take place.

With respect to the second potential condition, controller 140 may be configured to use UWB transceivers 132 to check for the presence of a caregiver badge 142 or tag 188 within a close proximity of patient support apparatus 20 (such as, for example about 1-3 meters, although other distances may be used). If no such caregiver-worn badge 142 or tag 188 is found in this vicinity, controller 140 may conclude that patient support apparatus 20 is not being touched by a caregiver, and therefore no caregivers will be exerting any forces on patient support apparatus 20 that would otherwise be detected by scale system 144 (and which would interfere with the zeroing process). Controller 140 may also conclude that no such touching of patient support apparatus 20 by a caregiver will be happening in the next few seconds when the automatic-zeroing process may take place.

With respect to the third potential condition, controller 140 may be configured to check the status of brake 250 onboard patient support apparatus. When brake 250 is activated, one or more of the wheels 24 of patient support apparatus 20 is prevented from rolling, thereby preventing patient support apparatus 20 from moving during the zeroing process. This may help prevent noise from being injected into the scale zeroing process.

With respect to the fourth potential condition, controller 140 may be configured to check to see if an onboard obstruction detection system 252 is currently detecting an obstruction or not. If it is currently detecting an obstruction, the force of the obstruction against the patient support apparatus 20 may change the readings of the force sensors of scale system 144, thereby making it undesirable to carry out a zeroing process at that time. Obstruction detection system 252 may take on a variety of forms. In some embodiments, additional sensors are added to patient support apparatus 20 that are specifically designed to detect impacts with object. In other embodiments, the obstruction detection system 252 may utilize the force sensors scale system 144 and/or exit detection system 136. One example of an obstruction detection system that may be incorporated into patient support apparatus 20 is the obstruction detection system disclosed in commonly assigned U.S. patent application Ser. No. 63/413,804 filed Oct. 6, 2022, by inventors Michael W. Graves et al. and entitled PATIENT SUPPORT APPARATUS WITH OBSTRUCTION DETECTION, the complete disclosure of which is incorporated herein by reference.

With respect to the fifth potential condition, controller 140 may be configured to check to see what the current height of litter frame 28 is. In some constructions of patient support apparatus 20, the force sensors of scale system 144 change their outputs when litter frame 28 is lowered to its lowest, or nearly lowest, height. Accordingly, controller 140 may be configured to check to see if litter frame 28 is currently above a threshold height. The particular value of the threshold height will depend upon the geometry and construction of the particular embodiment of the patient support apparatus 20. In general, the particular value will be whatever height value is determined to not interfere with obtaining weight readings via scale system 144 that are accurate enough for carrying out the zeroing process.

With respect to the sixth potential condition, controller 140 may be configured to determine whether patient support apparatus 20 is currently operating on battery power, or whether it is currently connected to a mains power supply via a power cable (e.g. an electrical outlet). If patient support apparatus 20 is not currently connected to a mains power supply via a power cable, there is a chance that the power cable may be draped over patient support apparatus 20, or otherwise set on patient support apparatus 20 in such a way that the weight of the cable is detectable by scale system 144, in which case it may be undesirable to carry out a zeroing process.

With respect to the seventh potential condition, controller 140 may be configured to monitor the outputs of the force sensors of scale system 144 for a predetermined amount of time (e.g. on the order of several seconds) to ensure that no disturbances above an expected noise threshold (or above a background noise level) are currently being detected. Such disturbances could interfere with the scale zeroing process. Accordingly, controller 140 may be configured to look for stable force sensor readings for a predetermined amount of time before allowing an automatic zeroing, and if not found, to not carry out the automatic zeroing process.

With respect to the eighth potential condition, controller 140 may be configured to only automatically zero scale system 144 if the weight readings from scale system 144 have drifted, or otherwise changed, by more than a threshold amount since the scale system was last zeroed. In other words, scale system 144 may be configured to take a current weight reading when no patient is present (empty-patient weight) and controller 140, if there is no equipment weight log, may determine if that weight deviates from zero by more than a first threshold. Alternatively, if controller 140 maintains an equipment weight log, controller 140 may be configured to compare the empty-patient weight reading to the total weight of the equipment in the equipment weight log and see if the difference (if any) is greater than the first threshold. In either instance, the first threshold may be on the order of 100 to 300 milligrams. If the empty-patient weight is more than the first threshold (no equipment log present), or if the difference between the equipment log weight and the empty-patient weight differs by more than the threshold (an equipment log is present), controller 140 is configured to not automatically zero scale system. This ensures that controller 140 zeroes, or re-zeroes, scale system 144 only when its weight readings have drifted, or otherwise moved, by more than the first threshold. Other thresholds beside 100 to 300 milligrams can, of course, be used.

In some embodiments, controller 140 may also check the value of the weight detected by scale system 144 prior to carrying out an automatic zeroing process in order to determine if a patient—who may not have a UWB tag 188 or badge 142—is present on patient support apparatus 20. In such embodiments, if scale system detects a weight of more than a threshold that is indicative of a potential patient on patient support apparatus 20, controller 140 may be configured to not carry out an automatic zeroing. The particular value of the threshold may vary, but should generally be selected to be higher than the greatest amount of equipment weight expected to be placed on patient support apparatus 20 but less than the weight of the smallest expected patient. In some embodiments, the weight threshold may be in the range of fifteen to thirty kilograms, although other values may be used.

It will be understood that the aforementioned eight potential conditions for carrying out an automatic zeroing of scale system 144 are not all necessary conditions for the automatic zeroing process in all embodiments of patient support apparatus 20. That it, some embodiments of patient support apparatus 20 may only require a single one of these conditions, while other embodiments may require more than one condition but less than all eight. Still further, one or more other conditions may be required in some embodiments of patient support apparatus 20, either in addition to, or in lieu of, one or more of these eight conditions. Accordingly, the aforementioned eight conditions may be mixed and matched with each other in any manner in different embodiments.

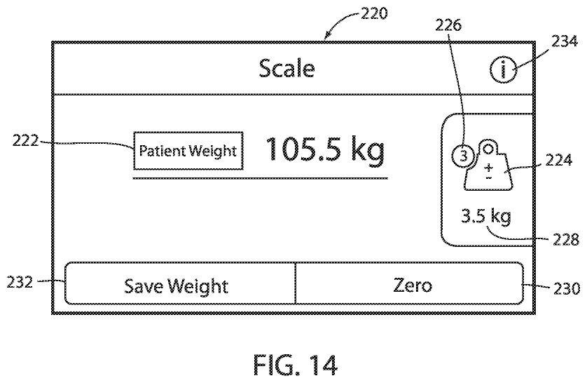

In some embodiments, controller 140 of patient support apparatus 20 may be configured to only allow a manual zeroing of scale system 144 if an authorized individual is detected within the vicinity of patient support apparatus 20. In such embodiments, controller 140 prevents unauthorized individuals from manually zeroing patient support apparatus 20. This is explained in more detail below with respect to FIG. 14.

FIG. 14 illustrates a scale screen 220 that controller 140 may be configured to display on display 52 in response to the user pressing on scale control 58c. Scale screen 220 includes a patient weight indicator 222, a weight log icon 224, an object number 226, a total object weight indicator 228, a manual zero control 230, a save weight control 232, and an information icon 234. Patient weight indicator 222 indicates the patient weight as currently measured by scale system 144, which is determined by subtracting the weight detected by scale system 144 at the time it was last zeroed from the current total weight detected by the force sensors of scale system 144. In some embodiments of patient support apparatus 20, controller 140 may be configured to maintain an equipment weight log of non-patient objects that are added to, or removed from, patient support apparatus 20. In such embodiments, the patient weight indicator 222 is determined by subtracting the weight of any objects that have been placed on patient support apparatus 20 and entered by controller 140 into the equipment weight log from the current total weight detected by the force sensors of scale system 144 (after the force sensors have been zeroed).

Log icon 224 is an icon that corresponds to the aforementioned weight log that may be maintained by controller 140 in some embodiments. That is, log icon 224 corresponds to a weight log that controller 140 may maintain of all of the objects that may be added to the litter frame 28 of patient support apparatus 20. If the user presses on weight log icon 224, controller 140 is configured to display a screen that provides more information about the weight log, such as a screen like those shown in FIGS. 8-11, 13, 15, and/or 16 of commonly assigned U.S. patent application Ser. No. 63/255,211 filed Oct. 13, 2021, by inventors Sujay Sukumaran et al. and entitled PATIENT SUPPORT APPARATUS WITH AUTOMATIC SCALE FUNCTIONALITY, the complete disclosure of which is incorporated herein by reference. Object number 226 indicates the number of objects that are currently in the weight log. Thus, in the example shown in FIG. 14, the weight log currently has three objects recorded therein that are positioned on litter frame 28. Total object weight indicator 228 indicates the total weight of the objects currently stored in the weight log. Thus, in the example of FIG. 14, the three objects stored in the weight log have a total cumulative weight of 3.5 kg, as indicated by indicator 228.

Screen 220 (FIG. 6) also includes a save weight control 232 and a manual zero control 230. If the user presses on the save weight control 232, controller 140 is configured to store the currently measured patient weight (as indicated by indicator 222) within memory 134 of patient support apparatus 20. If the user presses on manual zero control 230 (FIG. 6), controller 140 is configured to zero (i.e. tare) the scale system. That is, in response to control 230 being activated by a user, controller 140 takes a snapshot of the current total weight readings recorded by the force sensors of scale system 144 and considers that weight value to be equal to the empty weight of patient support apparatus 20 (i.e. the weight on the scale system when no patient is present and no objects of interest are present). Typically, a caregiver uses control 230 to zero the scale system when the patient is absent, but a mattress, a pillow, bedding, and other standard items (if any) are present on the patient support apparatus 20. In this manner, the weight of the mattress, pillow, bedding, etc., as well as the structural weight of the support deck 30 and litter frame 28, are zeroed out. In some embodiments, after the user has zeroed the scale system using control 230, controller 140 is configured to erase any items that were stored in the weight log, while in other embodiments, controller 140 is configured to maintain the items that were stored in the weight log.

Scale screen 220 (FIG. 6) may also include an information icon 234. Controller 140 is configured to display additional information about the scale system when icon 234 is pressed by a caregiver. In some embodiments, the additional information includes any one or more of the following items: the last time the scale system was zeroed (whether using control 230 or automatically, as described above), a current software version, a date when the scale system was last calibrated, an indication of whether the scale is automatically detecting the addition and removal of objects or not, and/or information regarding the scale's compliance with, and/or class within, the Non-Automatic Weighing Instruments (NAWI) scale standards required by the European Union and/or their member states.

In some embodiments, controller 140 is configured to automatically disable manual zero control 230 unless UWB transceivers 132 detect the presence of an authorized individual (via their respective badge 142 or tag 188) within the vicinity of patient support apparatus 20. The “vicinity” refers to a volume of space 152 that a caregiver would expect to be located in at when pressing on manual zeroing control 230. If controller 140 does not detect a badge 142 or tag 188 that corresponds to an authorized individual, controller 140 continues to disable zero control 230. This is true regardless of whether any unauthorized individuals are detected within the vicinity or outside of the vicinity. If controller 140 detects an authorized individual, but their badge 142 or tag 188 is not positioned within the volume of space 152 corresponding to the “vicinity,” controller 140 continues to disable zero control 230. If controller 140 detects the badge 142 or tag 188 of an authorized individual within the vicinity, then controller 140 enables control 230 and allows that authorized individual to zero scale system 144, if desired.

In some embodiments, controller 140 consults server 84 to determine if an individual is an authorized individual or an unauthorized individual. In such embodiments, controller 140 sends server 84 the ID 166 or 180 of the badge 142 or tag 188, respectively, that it detects within the vicinity of patient support apparatus 20 using UWB transceivers 132. Server 84 maintains a listing, or has access to a listing, of the IDs of those individuals who are authorized to zero scale system 144. This listing is populated by an administrator, or other authorized individual, of the healthcare facility within which patient support apparatus 20 is located. Server 84 then reports back to patient support apparatus 20 data indicating whether the individual is an authorized or unauthorized individual, and controller 140 proceeds to enable or disable zero control 230 accordingly.

In some embodiments, controller 140 is configured to take into account additional conditions when determining whether to enable or disable zero control 230. For example, in some embodiments, if controller 140 detects an equipment tag 188 at a location indicative of equipment currently being located on patient support apparatus 20, controller 140 may be configured to disable manual zero control 230. Similarly, if controller 140 detects a patient tag 188 at a location indicative of patient support apparatus 20 currently being occupied by a patient, controller 140 may also disable manual zero control 230. In some embodiments, controller 140 may take into consideration any one or more of the eight conditions previously mentioned when performing autozeroing, and if any of those eight conditions are not met, controller 140 may be configured to automatically disable zero control 230.

In some embodiments, controller 140 may be configured to communicate with one or more off-board sensors and use those outputs to determine whether to auto-zero scale system 144, and/or to determine whether to enable or disable zero control 230. For example, in some embodiments, controller 140 may be configured to communicate with a bathroom door sensor that detect movement of a bathroom door within a patient's room. Using the outputs of the force sensors of scale system 144, as well as information about when the position of the bathroom door changes, controller 140 may be configured to detect when the patient has exited patient support apparatus 20 to use the restroom. At this time, controller 140 may then proceed to perform an automatic zeroing of scale system 144. When performing this automatic zeroing, controller 140 may utilize any one or more of the aforementioned eight conditions (or others), or it may skip using any of these eight conditions (and/or other conditions).

In some embodiments, controller 140 and/or server 84 may be configured to remind the caregiver to position one or more pieces of equipment or other devices on patient support apparatus 20 prior to the caregiver manually zeroing scale system 144. In such embodiments, controller 140 may communicate with server 84 which, in turn, may communicate with EMR server 92 to determine what procedure(s) and/or treatment(s) have been prescribed for the particular patient who is assigned to patient support apparatus 20. From this information, server 84 and/or controller 140 may consult a database (stored internally, or on another accessible server of network 80) that relates which pieces of equipment and/or devices are to be used for that particular treatment and/or procedure. From this information, controller 140 may use UWB transceivers 132 to determine if such equipment and/or devices are positioned on patient support apparatus 20 and, if not, present a reminder on display 52 to the caregiver to place such equipment and/or devices on patient support apparatus 20 prior to zeroing scale system 144. In this manner, the caregiver is saved from the extra trouble of having to re-zero scale system 144 after the equipment or device is placed on patient support apparatus 20.

In any of the embodiments disclosed herein, server 84 and/or one or more of the electronic devices 98 may be configured to additionally execute a caregiver assistance software application of the type described in the following commonly assigned patent applications: U.S. patent application Ser. No. 62/826,97, filed Mar. 29, 2019 by inventors Thomas Durlach et al. and entitled PATIENT CARE SYSTEM; U.S. patent application Ser. No. 16/832,760 filed Mar. 27, 2020, by inventors Thomas Durlach et al. and entitled PATIENT CARE SYSTEM; and/or PCT patent application serial number PCT/US2020/039587 filed Jun. 25, 2020, by inventors Thomas Durlach et al. and entitled CAREGIVER ASSISTANCE SYSTEM, the complete disclosures of which are all incorporated herein by reference. That is, server 84 may be configured to share with one or more electronic devices 98 any of the information shared with the electronic devices disclosed in these aforementioned patent applications. Thus, for example, server 84 may be configured to not only share the location of patient support apparatuses 20 (and any devices that may be associated with them) with electronic devices 98, but it may also forward patient data to electronic devices 98 from one or more associated devices 100 (e.g. vital sign data, infusion pump data, etc.), patient support apparatus status data (e.g. current siderail position, bed exit status, brake status, motion lockout status, height status, scale data, etc.) badge 142 data, and/or caregiver rounding data (e.g. when the last rounding was performed for a particular patient, when the next rounds are due, etc.).

In some embodiments, locator units 60 may include additional information stored therein that is shared with patient support apparatus 20 when patient support apparatus 20 becomes associated with the locator unit 60. Such additional information may include location information identifying the relative position of the locator unit 60 with respect to one or more other locator units 60 that are positioned nearby. Additionally or alternatively, the locator units 60 may include information regarding the thickness and/or materials of the wall 62 to which it is attached, wherein such information provides an indication to the patient support apparatus 20 of the amount of attenuation that UWB signals will likely experience when traveling through that wall. Additionally or alternatively, the locator units 60 may include information identifying their general location within the healthcare facility (e.g. room 400, bay A of room 302, hallway X, maintenance area Y, radiology department, emergency department, etc.) and/or information identifying a more specific location of the locator units 60 within the healthcare facility (e.g. a set of X, Y, Z coordinates in a frame of reference that includes all, or a portion of, the healthcare facility; a height on the wall 62, a distance from one or more landmarks and/or architectural features within the healthcare facility, and/or other more specific information). In some embodiments, patient support apparatus 20 is adapted to utilize this information to determine its location within the healthcare facility and/or to determine whether it is positioned on the same side of the wall 62 as a particular locator unit 60. In some embodiments, patient support apparatus 20 and/or locator units 60 include any of the same structures, functions, and/or features of any of the patient support apparatuses and/or wall units disclosed in commonly assigned U.S. patent application Ser. No. 63/245,245 filed Sep. 17, 2021, by inventors Kirby Neihouser et al. and entitled SYSTEM FOR LOCATING PATIENT SUPPORT APPARATUSES, the complete disclosure of which has already been incorporated herein by reference.

It will also be understood that patient support apparatuses 20 and/or server 84 need not include all of the functionality described herein. That is, in some embodiments, for example, patient support apparatuses 20 may be adapted to carry out the automatic determination of which caregiver added a weight to patient support apparatus 20, but might not include the ability to automatically determine when they have been cleaned or are ready to be cleaned, the ability to automatically zero scale system 144, and/or the ability to enable and disable the manual zero control 230. Similarly, a patient support apparatus 20 that is able to carry out any one or more of these latter functions may, or may not, be able to carry out the automatic determination of which caregiver added a weight to patient support apparatus 20. Accordingly, the features and functions of patient support apparatus 20 described herein may be combined together in any fashion, or they may be implemented separately in a particular patient support apparatus 20.

The patient support apparatuses 20 and/or server 84 described herein may also be modified to carry out any one or more of the functions of the patient support apparatuses and patient support apparatus server described in the following commonly assigned U.S. patent applications: U.S. provisional patent application 63/356,061, filed Jun. 28, 2022, by inventors Krishna Bhimavarapu et al. (P-680) and entitled BADGE AND PATIENT SUPPORT APPARATUS COMMUNICATION SYSTEM; and/or U.S. provisional patent application 63/356,065, filed Jun. 28, 2022, by inventors Jerald A. Trepanier et al. (P-681) and entitled BADGE AND PATIENT SUPPORT APPARATUS COMMUNICATION SYSTEM, the complete disclosures of both of which are incorporated herein by reference.

It will be understood that the general difference between a device 100 and an object 190, as described herein, is the presence of one or more UWB transceivers 178 that are built into the device 100. Objects 190 generally do not include such UWB transceivers. Tags 188 are therefore added to objects 190 in order to give them the ability to communicate with the UWB transceivers 132 onboard patient support apparatus 20 (and/or the UWB transceivers 126, 126a of locator units 60). Any object 190 could therefore be the same as any device 100 minus its UWB transceiver 178, and any device 100 could be the same as an object 190 with a UWB tag 188 attached to it. Tags 188 can also be used without an attached object 190, such as when they are worn by patients or other individuals.

It will also be understood by those skilled in the art that the use of the term “transceiver” throughout this specification is not intended to be limited to devices in which a transmitter and receiver are necessarily within the same housing, or share some circuitry. Instead, the term “transceiver” is used broadly herein to refer to both structures in which circuitry is shared between the transmitter and receiver, and transmitter-receivers in which the transmitter and receiver do not share circuitry and/or a common housing. Thus, the term “transceiver” refers to any device having a transmitter component and a receiver component, regardless of whether the two components are a common entity, separate entities, or have some overlap in their structures.

Various additional alterations and changes beyond those already mentioned herein can be made to the above-described embodiments. This disclosure is presented for illustrative purposes and should not be interpreted as an exhaustive description of all embodiments or to limit the scope of the claims to the specific elements illustrated or described in connection with these embodiments. For example, and without limitation, any individual element(s) of the described embodiments may be replaced by alternative elements that provide substantially similar functionality or otherwise provide adequate operation. This includes, for example, presently known alternative elements, such as those that might be currently known to one skilled in the art, and alternative elements that may be developed in the future, such as those that one skilled in the art might, upon development, recognize as an alternative. Any reference to claim elements in the singular, for example, using the articles “a,” “an,” “the” or “said,” is not to be construed as limiting the element to the singular.

1. A patient support apparatus system comprising a patient support apparatus and a server, wherein the patient support apparatus comprises:
a support surface adapted to support a patient;
a plurality of ultra-wideband transceivers;
a display;
a network transceiver adapted to communicate with the server; and
a controller adapted to use radio frequency (RF) communication between the plurality of ultra-wideband transceivers and a badge worn by a worker to determine a position of the badge relative to the patient support apparatus, the controller further adapted to receive a badge identifier from the badge; to determine if the badge is positioned inside or outside of a volume of space; and, if the badge is positioned inside the volume of space, to transmit the badge identifier to the server; and
wherein at least one of the controller or the server is adapted to perform the following: determine if the badge is associated with a cleaning worker, and if the badge is associated with a cleaning worker, the controller is adapted to automatically display a cleaning screen on the display.

2. The patient support apparatus system of claim 1 wherein the cleaning screen includes instructions for cleaning the patient support apparatus.

3. The patient support apparatus system of claim 1 wherein at least one of the controller and the server is adapted to perform the following: (i) measure a length of time the badge remains inside the volume of space; (ii) compare the measured length of time to a threshold amount of time; and (iii) if the measured length of time exceeds the threshold amount of time, determine that the patient support apparatus has been cleaned.

4. The patient support apparatus system of claim 3 wherein, after the patient support apparatus has been determined to have been cleaned, the controller is adapted to determine if a scale system onboard the patient support apparatus has been zeroed or not.

5. The patient support apparatus system of claim 3 wherein, after the badge has been determined to be associated with a cleaning worker, the controller is further adapted to automatically display a bed zeroing reminder screen on the display.

6. The patient support apparatus system of claim 5 wherein the controller is adapted to automatically display the bed zeroing reminder screen on the display a predetermined amount of time after the badge has been determined to be associated with a cleaning worker.

7. The patient support apparatus system of claim 5 wherein the controller is adapted to display the bed zeroing reminder screen on the display a predetermined amount of time after the patient support apparatus has been determined to have been cleaned.

8-19. (canceled)

20. A patient support apparatus system comprising a patient support apparatus and a server, wherein the patient support apparatus comprises:
a support surface adapted to support a patient;
a plurality of ultra-wideband transceivers;
a display;
a network transceiver adapted to communicate with the server; and
a controller adapted to use radio frequency (RF) communication between the plurality of ultra-wideband transceivers and a badge worn by a worker to determine a position of the badge relative to the patient support apparatus, the controller further adapted to receive a badge identifier from the badge; to determine if the badge is positioned inside or outside of a volume of space; and, if the badge is positioned inside the volume of space, to transmit the badge identifier to the server; and
wherein at least one of the controller or the server is adapted to perform the following: (i) determine if the badge is associated with a cleaning worker; (ii) measure a length of time the badge remains inside the volume of space; (iii) compare the measured length of time to a threshold amount of time; and (iv) if the measured length of time exceeds the threshold amount of time, determine that the patient support apparatus has been cleaned.

21. The patient support apparatus system of claim 20 wherein, if the badge is associated with a cleaning worker, the at least one of the controller or server is further adapted to automatically display a cleaning screen on the display, the cleaning screen including instructions for cleaning the patient support apparatus.

22. (canceled)

23. The patient support apparatus system of claim 20 wherein, after the patient support apparatus has been determined to have been cleaned, the controller is adapted to determine if a scale system onboard the patient support apparatus has been zeroed or not.

24-26. (canceled)

27. The patient support apparatus system of claim 23 wherein at least one of the controller or the server is adapted to automatically send a reminder message to the badge if the scale system onboard the patient support apparatus has not been zeroed.

28-33. (canceled)

34. The patient support apparatus system of claim 20 wherein the at least one of the controller or the server is adapted to determine that the patient support apparatus has been cleaned only if an additional condition has been met in addition to the measured length of time exceeding the threshold amount of time.

35. The patient support apparatus system of claim 34 wherein the additional condition includes at least one of the following:
(a) a lack of patient weight detected by a scale system integrated into the patient support apparatus;
(b) a receipt of data from an Admission, Discharge and Transfer server indicating that the patient assigned to the patient support apparatus has been discharged from a healthcare facility in which the patient support apparatus is located;
(c) a lack of an ultra-wideband tag worn by the patient being detected inside the volume of space by the ultra-wideband transceivers; or
(d) a detection of a second badge inside the volume of space within a defined amount of time prior to the badge being positioned inside the volume of space.

36-39. (canceled)

40. A patient support apparatus system comprising a patient support apparatus and a server, wherein the patient support apparatus comprises:
a support surface adapted to support a patient;
a plurality of ultra-wideband transceivers;
a network transceiver adapted to communicate with the server; and
a controller adapted to use radio frequency (RF) communication between the plurality of ultra-wideband transceivers and a badge worn by a worker to determine a position of the badge relative to the patient support apparatus, the controller further adapted to receive a badge identifier from the badge; to determine if the badge is positioned inside or outside of a volume of space; and, if the badge is positioned inside the volume of space, to transmit the badge identifier to the server; and
wherein at least one of the controller or the server is adapted to perform the following: (i) determine if the badge is associated with a transport worker; and (ii) if the badge is associated with a transport worker, determine that the patient support apparatus is ready to be cleaned.

41. The patient support apparatus system of claim 40 wherein the at least one of the controller or the server is adapted to determine that the patient support apparatus is ready to be cleaned only if an additional condition has been met.

42. The patient support apparatus system of claim 41 wherein the additional condition includes at least one of the following:
(a) a measured length of time the badge remains within the volume of space exceeding a threshold amount of time;
(b) a lack of patient weight detected by a scale system integrated into the patient support apparatus;
(c) a lack of an ultra-wideband tag worn by the patient being detected within the volume of space by the ultra-wideband transceivers;
(d) a receipt of data from an Admission, Discharge and Transfer server indicating that the patient assigned to the patient support apparatus has been discharged from a healthcare facility in which the patient support apparatus is located; or
(e) a lack of the badge in the volume of space.

43-46. (canceled)

47. The patient support apparatus system of claim 40 wherein the server is further configured to send a message to a cleaning server after the patient support apparatus has been determined to be ready to be cleaned.

48. The patient support apparatus system of claim 40 wherein the server is further configured to send a message to a second badge, wherein the second badge is associated with a cleaning worker responsible for cleaning the patient support apparatus.

49. The patient support apparatus system of claim 40 wherein the server is further configured to automatically transmit a message to the badge after the badge has moved outside of the volume of space.

50. The patient support apparatus of claim 49 wherein the message identifies a transportation assignment for the worker.

51-135. (canceled)
